(19) United States
(12) Patent Application Publication
(10) Pub. No.: US 2010/0069293 A1
(43) Pub. Date: Mar. 18, 2010

(54) POLYMERIC CARRIER COMPOSITIONS FOR DELIVERY OF ACTIVE AGENTS, METHODS OF MAKING AND USING THE SAME

(75) Inventors: Elijah M. Bolotin, Bothell, WA (US); Gerardo M. Castillo, Bothell, WA (US); ManShun Lai, Bothell, WA (US); Akiko Nishimoto-Ashfield, Seattle, WA (US)

Correspondence Address: WILSON SONSINI GOODRICH AND ROSATI / PHARMAIN LTD, 650 PAGE MILL ROAD, PALO ALTO, CA, 94304, US

(73) Assignee: PharmaIN Corporation, Seattle, WA (US)

(21) Appl. No.: 12/556,467

(22) Filed: Sep. 9, 2009

Related U.S. Application Data
(63) Continuation-in-part of application No. 11/428,803, filed on Jul. 5, 2006.
Application No. 11/428,803 is a continuation of application No. 10/378,100, filed on Feb. 27, 2003, now Pat. No. 7,138,105.
(60) Provisional application No. 60/360,350, filed on Feb. 27, 2002.

(51) Int. Cl. A61K 38/28; A61P 3/10; A61K 47/48

(52) U.S. Cl. 514/3; 525/54.1

(57) ABSTRACT

In part, the present invention is directed to compositions and methods of making compositions comprising a polymeric backbone, a chelating group, a metal ion, and an active agent with a metal binding domain. The compositions can optionally further comprise protective groups. In part, the present invention is directed to prolonging the blood circulation time of an active agent containing a metal binding domain by using a composition comprising a polymeric backbone with a protective group, a chelator, and a metal ion.

### CROSS-REFERENCE

This application is a continuation-in-part of U.S. patent application Ser. No. 11/428,803, filed on Jul. 5, 2006, which is a continuation of U.S. patent application Ser. No. 10/378,100 filed on Feb. 27, 2003, which issued as U.S. Pat. No. 7,138,105 on Nov. 21, 2006, which claims the benefit of U.S. Provisional Application No. 60/360,350 filed on Feb. 27, 2002. These applications are incorporated herein by reference in their entirety.

### STATEMENT AS TO FEDERALLY SPONSORED RESEARCH

This invention was made with the support of the United States government under Contract number 1 R43AI078539 by National Institute of Aging and Infectious Disease (NIAID). The U.S. Government may have certain rights in subject matter provided herein.

### BACKGROUND OF THE INVENTION

The development of new drugs, formulations and other systems for administration of physiologically active peptides and proteins and other therapeutics and materials is driven by the need to provide these peptides or proteins or other materials to achieve the desirable physiological effects. With respect to peptides and proteins, many of them have been observed to be unstable in the gastro-intestinal tract and therefore may need to be stabilized or protected or delivered via systemic circulation. In addition, peptides and proteins that have low molecular masses tend to have short biological half-lives due to their efficient removal from systemic circulation via kidneys and reticuloendothelial system. For example, a fraction of these peptides and proteins can also be removed via reticulo-endothelial uptake due to recognition by monocyte/macrophages or as a result of opsonization by complement components. Many peptides and proteins can also lose their activity in vivo due to proteolysis (peptide bond cleavage).

In part to circumvent these undesirable effects, a drug delivery system may be used. There are drug delivery strategies that can be useful for peptide and protein delivery in vivo. First, a continuous systemic infusion of drug via a pump can be employed. This strategy is proven efficient in clinical practice but may be impractical for outpatients requiring high levels of mobility, associated disadvantages of quality of life and potential intravenous (I.V.) line infections.

Second, peptides and proteins can be included in an implantable pump comprised of a capsule with a membrane allowing diffusion of the drug, for example, at a desirable release rate. Due to limited volume of these capsules, peptides and proteins are often used in a concentrated formulation, which leads to a loss of solubility due to aggregation and potential loss of specific activity. In most cases, the drug is usually released into the extracellular space and distributed in lymphatics. Overall concentration of peptide or protein may be affected by local lymph node activity and the efficacy of lymph node drainage of the implantation site. There is also a potential of host reaction to capsule material but in general, this side effect is infrequent.

Third, the drug release system can be made biodegradable as a result of encapsulation or inclusion into degradable drug delivery vehicles or carriers, e.g. polymeric matrices, particles or membrane vesicles (liposomes). These delivery systems are usually either implantable or injectable. Implantable drug delivery systems are often placed under the epidermis where the components of the system are usually slowly degraded as a result of biological activity of surrounding cells (i.e. as a result of the release of enzymes degrading chemical bonds that hold these implants together).

In part, the present invention is directed towards novel drug delivery systems, comprising polymer composition with a chelator, a chelated transition metal ion, and an active agent coordinately bonded to the metal ion, and methods of making and using the same

### SUMMARY OF THE INVENTION

The present invention is directed to the composition and use of metal bridges to connect a backbone polymer and an active agent which can be an active agent capable of binding metal. The metal bridge is formed by the chelating moiety covalently bonded to the polymeric backbone, a transition metal ion, and a metal binding domain coordinately bonded to the metal ion and covalently bonded or naturally part of to the active agent. The subject compositions can provide a means of achieving sustained release of the active agent after administration to a patient. As used herein, a “metal bridge” comprises the chelating moiety pendant to the backbone polymer (the polymer can be branched or unbranched), the metal ion chelated to the chelating moiety, and the metal binding domain (MBD) of an active agent which is exemplified by a peptide, a protein, an oligonucleotide, a polynucleotide or a small molecular weight drug. As used herein, a “metal binding domain” is a portion of a molecule capable of coordinate bonding with metal and the term “chelating group” or “chelating moiety” is a subgroup of metal binding domain that is a moiety or a group that contains two or more pairs of electrons (Lewis bases) capable of having two or more coordinate bonds with a metal ion and the said coordination bonds are not separated or flanked by the backbone polymer. The word group or moiety in “chelating group or chelating moiety” refers to a cluster of covalently bonded atoms linked to the backbone polymer by a single bond. Although the term “chelating” means “claw”, indicating that it is a claw-shaped structure that can claw and hold metal ion, for the purpose of this specification “chelating group” also include a circular structure that can form at least two coordination bonds with a metal ion. These include circular structure such as heme and Trimethyl-1,4,7-triazacyclononane and the likes (see below). The metal binding domain of the active agent may or may not be a chelating moiety. The composition of the present invention has a metal that is chelated to the chelating moiety that is pendant to the backbone polymer and the metal ion is simultaneously coordinately bonded to an active agent (a peptide, a protein, an oligonucleotide, a polynucleotide or a small molecular weight drug). It may be the case that the metal bridge may comprise more than a single metal ion (i.e., multiple metal ions) with bridging ligands, provided that the chelating moiety of the backbone and MBD of the active agent are capable of being connected through the metal ions and bridging ligand. It may be the case in an active agent molecule that the metal binding domain is not a chelating moiety but is able to coordinate bond with the metal. It may also be the case that to further strengthen the binding a chelating moiety may optionally be added to a peptide, a protein, an oligonucleotide, a polynucleotide or a small molecular weight drug. The term “carrier” for the purpose of this disclosure is any composition comprising a backbone polymer (branched or linear polymer) with chelating moiety covalently attached to the backbone polymer. For the purpose of this specification, the term polymeric backbone “does not” include polypeptides and proteins that are naturally occurring or have biological activity. Biological activity means that it can bind to a cellular receptor and cause transmission of signal inside the cell via a second messenger. Despite the fact that peptide and proteins has repeating amide bonds, it does not have repeating monomeric chemical units. Naturally occurring peptides or proteins are naturally found in nature.

The present invention relates composition comprising (i) a polymeric backbone with monomeric units (ii) a chelating group covalently linked to the monomeric unit, (iii) a transition metal ion, iv) a active agent (a peptide, a protein, nucleotide, oligonucleotide, or a drug with molecular weight of less than 2000 Daltons) coordinately bonded to the transition metal ion, and (v) optionally, a protective chain covalently bonded to the backbone.

In one embodiment of the present invention, the polymeric backbone is selected from polylysine, polyornithine, polyarginine, polyaspartic acid, polyglutamic acid, polyserine, polythreonine, polycysteine, polyglycerol, polyethyleneimines, polyallylamine, chitosan, hyluronan, natural saccharides, aminated polysaccharides, aminated oligosaccharides, polyamidoamine, polyacrylic acids, polyalcohols, sulfonated polysaccharides, sulfonated oligosaccharides, carboxylated polysaccharides, carboxylated oligosaccharides, aminocarboxylated polysaccharides, aminocarboxylated oligosaccharides, carboxymethylated polysaccharides, or carboxymethylated oligosaccharides.

In another embodiment of the present invention, the polymeric backbone has a molecular weight ranging from about 100 to about 1,000,000 Daltons. In further embodiment, the polymeric backbone is linear. In a further embodiment, the linear polymeric backbone has a molecular weight ranging from about 5,000 to about 100,000 Daltons. In a further embodiment, the linear polymeric backbone comprises poly amino acid or polyamine. In a further embodiment, the linear polymeric backbone comprises poly-lysine or polyallyamine backbone.

In another embodiment of the present invention, the polymeric backbone has a molecular weight ranging from about 100 to about 1,000,000 Daltons. In a further embodiment, the polymeric backbone is branched. In a further embodiment, the branched polymeric backbone has a molecular weight ranging from about 5,000 to about 100,000 Daltons. In a further embodiment, the branched polymeric backbone comprises polyamine. In a further embodiment, the branched polyamine backbone comprises polyethyleneimine.

In another embodiment of the present invention, the polymeric backbone is a co-polymer made up of two different polymers. In another embodiment of the present invention, the polymeric backbone is a co-polymer made up of two different polymers selected from polylysine, polyornithine, polyarginine, polyaspartic acid, polyglutamic acid, polyserine, polythreonine, polycysteine, polyglycerol, polyethyleneimine, polyallylamine, chitosan, hyluronan, natural saccharides, aminated polysaccharides, aminated oligosaccharides, polyamidoamine, polyacrylic acids, polyalcohols, carboxylated polysaccharides, carboxylated oligosaccharides, aminocarboxylated polysaccharides, aminocarboxylated oligosaccharides, carboxymethylated polysaccharides, or carboxymethylated oligosaccharides. In a further embodiment, the co-polymeric backbone has a molecular weight ranging from about 100 to about 1,000,000 Daltons. In a further embodiment, the co-polymeric backbone is made up of a linear and a branched polymer. In a further embodiment, the branched polymeric of the co-polymeric backbone is polyethyleneimine and the linear polymer is polyamino acid. In a further embodiment, the polyamino acid of co-polymeric backbone is polylysine.

In a further embodiment of the present invention, the chelating group covalently linked to the monomeric unit comprises a nitrogen-containing poly carboxylic acid. In another embodiment, the chelating group covalently linked to the monomeric unit is selected from a group consisting of: Trimethyl-1,4,7-triazacyclononane (TACN); 1,4,7,10-tetraazacyclododecane-N,N′,N″,N′″-tetraacetic acid; 1,4,7,10-tetraazacyclododecane-N,N′,N″-triacetic acid; 1,4,7-tris(carboxymethyl)-10-(2′-hydroxypropyl)-1,4,7,10-tetraazocyclodecane; 1,4,7-triazacyclonane-N,N′,N″-triacetic acid; 1,4,8,11-tetraazacyclotetra-decane-N,N′,N″,N′″-tetraacetic acid; 1,2-diaminocyclohexane-N,N,N′,N′-tetraacetic acid; bis(aminoethanethiol)carboxylic acid; diethylenetriamine-pentaacetic acid (DTPA); ethylenediamine-tetraacetic acid (EDTA); ethyleneglycoltetraacetic acid (EGTA); ethylene-bis(oxyethylene-nitrilo)tetraacetic acid; ethylenedicysteine; Imidodiacetic acid (IDA); N-(hydroxyethyl)ethylenediaminetriacetic acid; nitrilotriacetic acid (NTA); nitrilodiacetic acid (NDA); triethylenetetraamine-hexaacetic acid (TTNA); bisphosphonate or a polypeptide having the formula: (AₓH_{y})ₚ, wherein A is any amino acid residue, H is histidine, x is an integer from 0-6; y is an integer from 1-6; and p is an integer from 2-6.

It should be noted that the bisphosphonate above may be pamidronate, etidronate, alendronate, ibandronate, zoledronate, risendronate, and other derivatives of pamidronate.

In one embodiment of the present invention, the transition metal ion is selected from Co³⁺, Cr³⁺, Hg²⁺, Pd²⁺, Pt²⁺, Pd⁴⁺, Pt⁴⁺, R³⁺, Ir³⁺, R³⁺, Co²⁺, Ni²⁺, Cu²⁺, Zn²⁺, Cd²⁺, Mn²⁺, Fe³⁺, Fe²⁺, Tc, Au³⁺, Au⁺, Ag⁺, Cu⁺, MoO₂²⁺, Ti³⁺, Ti⁴⁺, CH₃Hg⁺, and Y⁺³. In another embodiment, the transition metal ion is selected Zn²⁺, Ni²⁺, Co²⁺, Fe²⁺, Mn²⁺, or Cu²⁺.

In another embodiment of the present invention, the optional protective side chain covalently bonded to the polymeric backbone has a molecular weight ranging from about 100 to about 1,000,000 daltons. In a further embodiment, the protective side chain is linear. In a further embodiment, the linear protective side chain has a molecular weight ranging from about 1,000 to about 25,000 daltons. In a further embodiment, the linear protective side chain comprises poly(ethylene glycol). In a further embodiment, the protective side chain comprises alkoxy poly(ethylene glycol). In a further embodiment, the protective side chain comprises methoxy poly(ethylene glycol) (MPEG).

In certain embodiments, the present invention may not require the attachment of chelator to a peptide, a protein, an oligonucleotide, a polynucleotide or a small molecular weight drug, in so much as an active agent may associate with metal ion by simple coordinate bonding without chelation (FIGS. 2 & 3). In addition to coordinate bonding, an active agent may further associate with a carrier via other non-covalent binding such as hydrophobic and/or electrostatic (including ionic and hydrogen bonding), van der Waal forces, or other weak interactions with the protective side chains, spacers, or, depending on the carrier, polymeric backbone. In a further embodiment, the active agent may further one or more metal binding domain that may be a chelating group selected from: trimethyl-1,4,7-triazacyclononane (TACN); N-(hydroxyethyl)ethylenediaminetriacetic acid; nitrilotriacetic acid (NTA); ethylene-bis(oxyethylene-nitrilo)tetraacetic acid; 1,4,7,10-tetraazacyclodo-decane-N,N′,N″,N′″-tetraacetic acid; 1,4,7,10-tetraaza-cyclododecane-N,N′,N″-triacetic acid; 1,4,7-tris(carboxymethyl)-10-(2′-hydroxypropyl)-1,4,7,10-tetraazocyclodecane; 1,4,7-triazacyclonane-N,N′,N″-triacetic acid; 1,4,8,11-tetraazacyclotetra-decane-N,N′,N″,N′″-tetraacetic acid; diethylenetriamine-pentaacetic acid (DTPA); ethylenedicysteine; bis(aminoethanethiol)carboxylic acid; triethylenetetraamine-hexaacetic acid; ethylenediamine-tetraacetic acid (EDTA); 1,2-diaminocyclohexane-N,N,N′,N′-tetraacetic acid; bisphosphonate (may be derived from pamidronate) or polypeptide. In a further embodiment, the polypeptide in the metal binding domain has the formula: (AxHy)p where A is any amino acid residue, H is histidine, x is an integer from 0-6; y is an integer from 1-6; and p is an integer from 2-6.

In the embodiments of the present invention, it is understood that not all active agents (represented by a peptide, a protein, a polynucleotide, an oligonucleotide, or a drug with molecular weight of less than 2000 Daltons) in a sample of the composition can be attached to the carrier through the metal ion, but that some portion of the active agent may be combined with the carrier. Likewise, it is understood that not all of the chelators attached to the carrier will chelate a metal ion, and that not all of the chelated metal ions will form a coordinate bond with an active agent.

In a further embodiment, the present invention relates to the above described composition wherein the polymeric backbone comprises polylysine, the metal binding domain comprises NTA, the metal ion is Ni²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polylysine, the metal binding domain comprises NTA, the metal ion is Zn²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polylysine, the metal binding domain comprises NTA, the metal ion is Cu²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polyethyleneimine, the metal binding domain comprises NTA, the metal ion is Ni²⁺, the active agent is selected from NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polyethyleneimine, the metal binding domain comprises NTA, the metal ion is Zn²⁺, the active agent is selected from NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polyethyleneimine, the metal binding domain comprises NTA, the metal ion is Cu²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polyallylamine, the metal binding domain comprises NTA, the metal ion is Ni²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polyallylamine, the metal binding domain comprises NTA, the metal ion is Zn²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises polyallylamine, the metal binding domain comprises NTA, the metal ion is Cu²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises co-polymer of polyethyleneimine and polylysine, the metal binding domain comprises NTA, the metal ion is Ni²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises co-polymer of polyethyleneimine and polylysine, the metal binding domain comprises NTA, the metal ion is Zn²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises co-polymer of polyethyleneimine and polylysine, the metal binding domain comprises NTA, the metal ion is Cu²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises co-polymer of polyethyleneimine and polyallylamine, the metal binding domain comprises NTA, the metal ion is Ni²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises co-polymer of polyethyleneimine and polyallylamine, the metal binding domain comprises NTA, the metal ion is Zn²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In a further embodiment, the present invention relates to the above described composition wherein the polymeric backbone comprises co-polymer of polyethyleneimine and polyallylamine, the metal binding domain comprises NTA, the metal ion is Cu²⁺, the active agent is selected from bisphosphonate-, NDA- or IDA-containing peptide, protein, polynucleotide, oligonucleotide, or drug with molecular weight of less than 2000 Daltons and the protective side chain comprises MPEG.

In another embodiment, the present invention relates to a pharmaceutical composition comprising any of the above described compositions. In a further embodiment, the pharmaceutical composition is an injectable composition.

In another embodiment, the present invention relates to a kit comprising a composition comprising: (i) a polymeric backbone (ii) a chelating moiety covalently linked or bonded to the backbone; (iii) a metal ion chelated to the chelating moiety by at least two coordinate bonds; (iv) an active agent with a MBD (which may or may not be a chelator) coordinately bonded to the metal ion; and optionally (v) a protective chain covalently linked or bonded to the backbone. Uses for such kits include, for example, therapeutic applications. Such kits may have a variety of uses, including, for example, imaging, targeting, diagnosis, therapy, vaccination, and other applications.

In another aspect, the compositions of the present invention may be used in the manufacture of a medicament for any number of uses, including for example treating any disease or other treatable condition of a patient. In still other aspects, the present invention is directed to a method for formulating compositions of the present invention in a pharmaceutically acceptable excipient.

The present invention provides a number of methods of making the subject compositions. Examples of such methods include those described in the exemplification below. These embodiments of the present invention, other embodiments, and their features and characteristics, will be apparent from the description, drawings and claims that follow.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary c metal ion bridge composition or carrier of the present invention. This diagram is not to limit the present invention to a linear polymeric backbone as branched polymers and co-polymers of branched and linear polymers are also part of the instant specification.

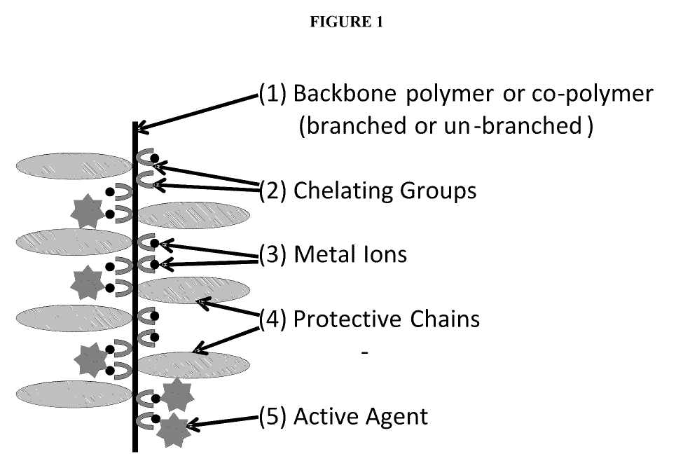

FIG. 2 depicts a graph showing the binding of human growth hormone (hrGH) to polymers in the presence of zinc and nickel cations. Size-separation on a Centricon YM-100 membrane demonstrates that approximately 1 mg of rhGH binds to 100 mg of PLPEGNTAZn (lot#20020105).

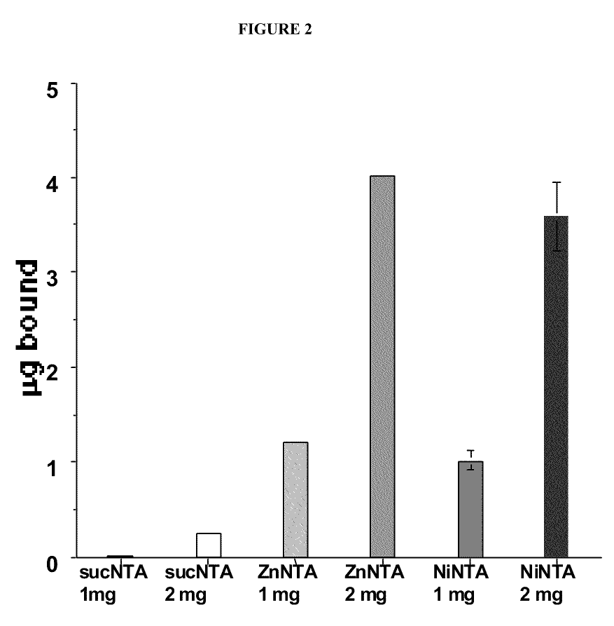

FIG. 3 depicts a chromatogram showing elution profiles of ¹²⁵I-labeled rhGH (squares) and an rhGH complex with PLPEGNTAZn (circles) on a SEC-5 size-exclusion HPLC column. The profile of time-dependent elution shows that a fraction of the complex of labeled hormone with PLPEGNTAZn (lot#20020105) elutes earlier than the free hormone, demonstrating a complex formation. The rhGH is dragged towards the void volume by the carrier containing metal chelate. This result is presented to demonstrate that the interaction of chelated metal and the metal binding domain of the protein is stable and can survive the gel permeation chromatography involving thousands of re-equilibrations (equal to the number of theoretical plates of the column) as the sample passes through the column. Weak interaction can cause the complex to dissociate resulting in an unaltered rhGH peak which is not observed in this case.

FIG. 4 depicts a bar-graph showing histidine tagged-GFP bound to PLPEGNTA (Ni or Zn salts), PLPEG (lot#20020101) or PLPEGSA (lot#20020102) after separation of bound complexes from free complexes in the presence or absence of blood plasma. The graph shows that complex formation with metal salts of PLPEGNTA (lot#20020103) is equally possible in the presence or absence of bulk protein of plasma. The same behavior is expected if an active agent is modified by the addition of a histidine metal binding domain.

FIG. 5 depicts a bar graph showing the levels of GFP in the plasma of animals injected with a histidine tagged-GFP (control); and complexes of histidine tagged-GFP with PLPEGNTAZn (lot#20020105) and PLPEGNTANi (lot#20020104). The graph shows higher in vivo levels of GFP in blood in the case of Ni-complex suggesting prolonged circulation of the histidine tagged-GFP bound to the PLPEGNTANi carrier. It is expected that if an active agent is modified in similar manner, a similar improvement is expected.

FIG. 6 depicts a carrier targeting inflammation and infection sites; the carriers of the present invention have a long-circulation half-life and can efficiently accumulate in sites of E. coli-induced inflammation and thus represent an alternative to inflammation-specific agents. For this experiment, male Sprague-Dawley rats were infected with previously frozen Escherichia coli (diluted in sterile isotonic saline to a final viable cell titer of 9×10⁸ organisms per 0.15 mL) in the posterior portions of the left thigh muscle. 3D maximum intensity projection MR images at 1, 12 and 24 hours after IV administration of gadolinium-labeled PLPEGDTPA are presented.

### DETAILED DESCRIPTION OF THE INVENTION

Definitions: For convenience, before further description of the present invention, certain terms employed in the specification, examples and appended claims which need further explanations are collected here. These definitions is expected to be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used to refer to one or to more than one (i.e., to at least one) of the grammatical object of the article. By way of example, “an element” means one element or more than one element.

The term “carrier” for the purpose of this invention refers composition of the present invention that comprises a polymer backbone with chelating moieties with or without associated metal ion and optionally with covalently attached protective chains.

The term “backbone” for the purpose of this invention refers to the structure comprising a polymer (linear or branched; polymer or co-polymer) from which the chelating moiety or group is covalently attached or linked.

The term “derivative” or “analog” as used herein refers to a compound whose core structure is the same as, or closely resembles that of, a parent compound, but which has a chemical or physical modification, such as a different or additional groups; the term includes co-polymers of parent compounds that can be linked to other atoms or molecules. The term also includes a peptide or protein with at least 50% sequence identity with the parent peptide or protein. The term also includes a peptide with additional groups attached to it, such as additional amino acid or chelating group, compared to the parent peptide. The term also includes a polymer with additional group attached to it, such as alkoxy group, compared to the parent polymer.

The term “a peptide, a protein, an oligonucleotide, a polynucleotide or a small molecular weight drug” is art recognized and may be isolated or synthetically prepared. Derivatives and fragments may also be isolated or synthetically prepared. It is possible that certain derivatives of an active agent may have several metal binding domains which may or may not be chelating moietie(s). An active agent derivative can be generated by truncation of the amino acid sequence or addition of other amino acids or functional groups such as chelating group.

The term “naturally-occurring” or “native”, as applied to an object, refers to the fact that an object may be found in nature. For example, a backbone that may be isolated from a source in nature and which has not been intentionally modified by man in the laboratory is naturally-occurring. The term “non-naturally-occurring” or “non-native” is as applied to an object that has been intentionally modified by man in the laboratory and not normally found in nature.

A “patient,” “subject” or “host” to be treated with the composition of the present invention may mean either a human or non-human animal. The term “mammal” is known in the art, and exemplary mammals include humans, primates, bovines, porcines, canines, felines, and rodents (e.g., mice and rats).

The term “pharmaceutically acceptable excipient” is art-recognized and refers to a pharmaceutically-acceptable material, composition or vehicle, such as a liquid or solid filler, diluent, solvent or encapsulating material, involved in carrying or transporting any supplement or composition, or component thereof, from one organ, or portion of the body, to another organ, or portion of the body. Each excipient must be “acceptable” in the sense of being compatible with the other ingredients of the supplement and not injurious to the patient. Some examples of materials which may serve as pharmaceutically acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “polymer” is a molecule (or macromolecule) composed of “repeating” structural units connected by covalent chemical bonds. This term includes polyamino acids, polyallyamine, polyetheleneimine, polysaccharides and other polymeric backbone mentioned in the instant specification. For the purpose of clarity of the instant specification, the term “polymeric backbone” or “backbone polymer” is a non-proteinaceous polymer. Proteinaceous means naturally occurring proteins or their derivatives which is not a homopolymer and has enzymatic or biological activity caused by its three dimensional conformation. Polyamino acid homopolymer such as polylysine is non-proteinaceous.

### General Introduction:

In part, the present invention is a carrier-based drug-delivery system comprising of a backbone polymer, a chelating molecule (interchangeably referred to as a chelator or chelating agent) covalently linked to the backbone, a metal ion chelated by the chelating molecule, and an active agent (such as but not limited to a peptide, a protein, a polynucleotide, an oligonucleotide, a drug, or a diagnostic agent) coordinately bonded to the metal ion. Optionally, the backbone contains protective group or shield (interchangeably referred to as protective chain, protective group, protective shield) comprising a hydrophilic group such as polyethylene glycol to protect the active agent. Protective chains can increase the overall hydrodynamic radius of the active agent and can result in prolonged circulation in the blood and can increase accumulation at sites of high vascular permeability.

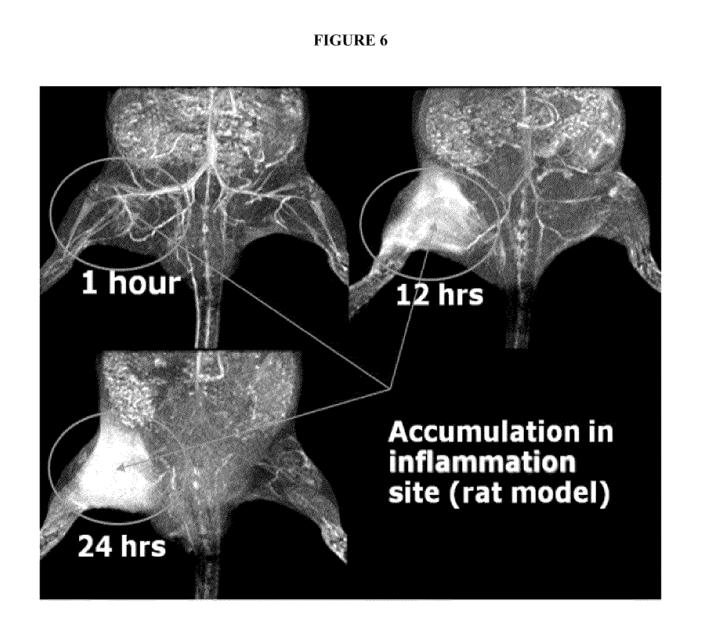

The carrier of the present invention confines to the circulation in vivo and permeates broken down or abnormal vascular barriers due to their high permeability levels. This was demonstrated in a model of bacterial inflammation of the muscle tissue in rats induced with E. coli (FIG. 6) The carrier could be used for early detection of leakage into the extra vascular space and specific targeting to the sites with increased vascular permeability, such as inflammation (see FIG. 6). Thus, increased accumulation of the present carrier at sites of inflammation will allow the carrier-associated-an active agent to accumulate at sites of infection.

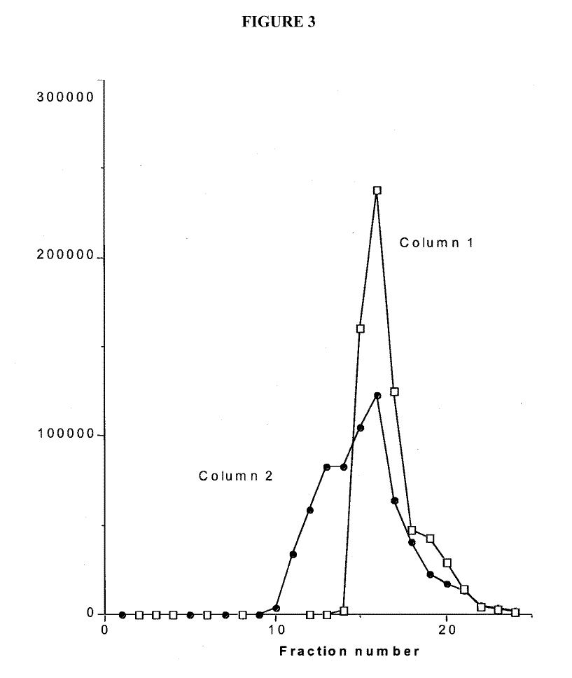

This association of an active agent (such as but not limited to a peptide, a protein, a polynucleotide, an oligonucleotide, a drug, or a diagnostic agent) to the polymeric backbone was accomplished using a metal bridge to connect the carrier backbone to a peptide, a protein, an oligonucleotide, a polynucleotide or a small molecular weight drug. This is applicable to unmodified peptide, protein, or drug or to modified peptide, protein, or drug or their derivatives that either maintained or enhanced metal coordination ability. For example, this is applicable to both His-Tagged—as well as native—proteins able to bind metal by coordinate bonding to the metals. FIG. 3 is size exclusion chromatogram demonstrating complex formation between a model protein (recombinant human growth hormone; rhGH) and a backbone containing zinc-chelate via a zinc bridge. One advantage of chelated metals attached to the carrier is to afford reversible binding of active agents (such as a peptide, a protein, a polynucleotide, an oligonucleotide, or a drug with molecular weight of less than 2000 Daltons) which are capable of forming coordination bonds with metal ions (e.g., Zn, Cu, or Ni). The coordinate bonding affords reversible dissociation of useful metal binding active agent from the polymeric backbone containing chelated metal.

Based on results presented, active agent binds to the chelator-metal complex of the carrier via metal coordination of perhaps one or more histidines in addition to other amino acids. Interactions may also have been facilitated by interactions with PEG chains and/or other components of the carrier. The design of the carrier is made in such a way that the associated an active agent would be protected by the polyethylene glycol chains from peptidases and antibodies. In addition, the association of an active agent with the high molecular weight carrier is expected to prolong its half life by preventing its excretion via renal ultrafiltration, uptake by antigen presenting cells, uptake by reticuloendothelial system.

In part, the present invention relates to a carrier comprising: (i) a polymeric backbone; (ii) a chelating moiety covalently bonded or linked to the backbone; (iii) a metal ion chelated to the chelating moiety; (iii) an active agent with a metal binding domain coordinately bonded to the metal ion; and optionally (iv) a protective chain covalently linked to the backbone. By way of a further embodiment an active agent may bind to a carrier by further means in addition to metal ion.

The compositions of the backbone of the carrier of the present invention include polymers and co-polymers of linear or branched structure or conjugates thereof.

In one example, a composition of the present invention comprises the linear polymeric backbone with degree of polymerization in the range of 2-10,000 to which independently and covalently linked are methoxypolyethylene glycol (mPEG) protective chains with a mass of 300-25,000 Daltons and chelating groups, where said protective chains and chelating groups are independently linked or pendant to the backbone. In another example, the degree of polymerization of the polymeric backbone is in the range of 25-1,000. In still another example, the degree polymerization of polymeric backbone is in the range of 50 to 300.

The chelating moiety of the present invention may include polycarboxylic acids containing nitrogen (such as iminodiacetic acid or IDA, nitrilodiacetic acid or NDA, nitrilotriacetic acid or NTA; EDTA; DTPA and the like) where at least one of carboxylic groups or the amino group may be utilized for covalent linking of the chelate or chelator to the backbone component of the carrier. The chelating moiety of the present invention may also be amine (primary or secondary) containing chelator where the amine may be utilized for covalent linking to the backbone component of the carrier (such as for example N,N-Bis(carboxymethyl)-lysine; Iminodiacetic acid and the like). The addition of metal ions to chelator can result in formation of coordinate complexes (metal-chelates) either at room temperature or at elevated temperatures. These metal-chelate complexes can coordinately bind to the metal binding domain of an active agent (such as a peptide, a protein, a polynucleotide, an oligonucleotide, or a drug with molecular weight of less than 2000 Daltons): added in a purified state; in water; in a buffer; or in the presence of bulk protein or blood plasma proteins. The addition can result in formation of drug-delivery compositions containing coordinate complexes formed between the metal-chelate and an active agent or derivatives. The amino acid sequence of active agent of the invention may include one or more histidines or cysteines which increase the stability of the complex formed with the compositions of the invention.

### Components of the Carrier of the Invention and Active Agents:

### A) Backbone Polymers/Co-Polymers:

In certain embodiments, the backbone polymers or backbone co-polymers of the subject compositions have molecular weights ranging from about 500 to 100,000, or alternatively about 10,000, 20,000, 30,000, 40,000, or 50,000, 60,000, 70,000, 80,000, or 90,000, and even more specifically between 5,000 to 50,000 Daltons. Number-average molecular weight (Mn) may also vary widely, but generally fall in the range of about 1,000 to about 120,000 Daltons or even from about 5,000 to about 70,000 Daltons or even from about 10,000 to about 50,000 Daltons. In one embodiment, Mn varies between about 8,000 and 45,000 Daltons. Within a given sample of a subject polymeric backbone, a wide range of molecular weights may be present. For example, molecules within the sample may have molecular weights which differ by a factor of 2, 5, 10, 20, 50, 100, or more, or which differ from the average molecular weight by a factor of 2, 5, 10, 20, 50, 100, or more. The number of monomer in the backbone polymer may vary from 10-mer to 1,000-mer, alternatively about 25, 50, 100, 150, 200, 250, 300, 350, 400, or 450-mer, and even more specifically between 100-mer to 250-mer. The number of monomer in the polymeric backbone generally determined the number of functional groups that can be modified to carry chelating moiety or protective chains.

The polymeric backbone is a non-proteinaceous homo- or heteropolymer with repeating monomers containing amino, carboxyl, hydroxyl, or thiol groups, and may be of natural or synthetic origin wherein the repeating monomeric groups can be covalently modified to contain chelating groups and optionally hydrophilic protective chains. The term “non-proteinaceous polyamino acid” as used herein refers to a polyaminoacid that is not naturally made by a living organism unless recombinantly engineered by human and does not have enzymatic or biological activity resulting from its three dimensional conformation. Preferably the polymeric backbone is polyamino acid which may have D- or L-chirality or both and more preferably a straight chain homopolymer. In one preferred embodiment, straight chain homopolymers include polylysine and polyornithine, polyarginine, polyglutamate, polyaspartate, polyserine, polythreonine, polytyrosine or any other amide linked homopolymer made from amino acids. The polymeric backbone may have a molecular weight of about 600-1,000,000 daltons, preferably 10,000-100,000 daltons. Other polymeric backbones with repeating modifiable functional groups may also be used such as those with repeating sulfhydryl(thiol), phosphate, and hydroxyl groups. Carbohydrate polymers and other synthetic polymers where monomers are non-biological may also be used as polymeric backbone. The polymeric backbone provides the multiple sites from where the chelating groups and hydrophilic protective chains can be attached.

Polysaccharides encompass disaccharides, oligosaccharides and larger polymers up to millions of Daltons. Polymeric backbone include polysaccharides, oligosaccharides and products chemically derived thereof, bearing modifiable carboxylic groups, alcohol groups or amino groups, which may be exemplified by: polyxylotol, galacturonic acid, glucuronic acid, mannuronic acid, hyaluronic acid, pectic acid, neuraminic acid, alginic acid, carrageenan; oxidized dextrans; aminated dextran, e.g. containing linked aminogroups. Polymeric backbone includes polysaccharides that is linear or branched that may be carboxylated or carboxymethylated. Polymeric backbone include polysaccharides reacted with derivatives of carbonic, dicarbonic, sulfuric, aminosulfuric, phosphoric acids with resultant linking of carboxylic, aminocarboxylic, carboxymethyl, sulfuric, amino or phosphate groups. Polymeric backbone includes polysaccharides obtained by chemical alteration of dextran, mannan, xylan, pullulan, cellulose, chytosan, agarose, fucoidan, galactan, arabinan, fructan, fucan, chitin, pustulan, levan or pectin. In addition these polysaccharides may be represented by heteropolymers or homopolymers of monosaccharides such as glucose, galactose, mannose, galactose, deoxyglucose, ribose, deoxyribose, arabinose, fucose, xylose, xylulose, ribulose. Polymeric backbone includes polymers (linear or branched) such as polyethyleneimine, polyamidoamine, polyallyamine, polyacrylic acid; polyalcohols (e.g. polyvinylalcohol) to which carboxylic, aminogroups, or alcohol groups are chemically linked and/or available for attachment of chelating groups. Some of these polymeric backbones are non-biological to which carboxylic, aminogroups, or alcohol groups are available for attachment of chelating groups.

In another embodiment, the polymer acting as the polymeric backbone may be poly(ethylene glycol) (PEG) with functional groups at the far-end making up the chelating group to which the metal ion coordinates and in turn coordinates the active agent. Schematically the embodiment may be represented by the following: PEG-chelator-Metal-MBD-Active agent. Alternatively, PEG may be functionalized along its backbone allowing chelator-Metal-MBD-Active agent moieties to be pendant to the backbone. This structure may also allow pendant protective chains as well.

### B) Metal Binding Domain:

In general, the metal binding domains (MBDs) used in the present invention contain a Lewis base moiety or functional group that encompasses numerous chemical moieties having a variety of structural, chemical and other characteristics capable of forming coordination bonds with a metal ion. The types of functional groups capable of forming coordinate complexes with metal ions are too numerous to categorize here, and are known to those of skill in the art. For example, such moieties will generally include functional groups capable of interaction with a metal center, e.g., heteroatoms such as nitrogen, oxygen, sulfur, and phosphorus. It should be noted that chelating group or moiety a subgroup of metal binding domain (MBD). Thus there are two types of MBD: a) chelating group or moiety, and b) non-chelating group or moiety but able to form coordinate bonding with metal. Both types are able to coordinate bond with metals. The nature of coordinate bonding is based on the fact that metal cations are almost always Lewis acids and are therefore able to bind various moieties that may serve as Lewis bases. In general, a moiety serving as a Lewis base can be a strongly acidic group prior to proton loss, (e.g., with a pKa less than about 7, and more preferably less than 5). Once proton is loss, it is a conjugate base that under the appropriate conditions is a strong enough Lewis base to donate an electron pair to a metal ion to form a coordinate bond. The degree of this Lewis acid-to-Lewis base (metal ion-to-metal binding domain) interaction is a function not only of the particular metal ion, but also of the coordinating moiety itself, because the latter may vary in the degree of basicity as well as in size and steric accessibility. Lewis basic moieties which may be included in the metal binding domain include: amines (primary, secondary, and tertiary) and aromatic amines, amino groups, amido groups, nitro groups, nitroso groups, amino alcohols, nitriles, imino groups, isonitriles, cyanates, isocyanates, hydroxyls, carbonyls (e.g., carboxyl, ester and formyl groups), aldehydes, ketones, ethers, carbamoyl groups, thiols, sulfides, thiocarbonyls (e.g., thiolcarboxyl, thiolester and thiolformyl groups), thioethers, and mercaptans. Illustrative of suitable metal binding domains include those chemical moieties containing at least one Lewis basic nitrogen, or oxygen atom or a combination of such nitrogen, and oxygen atoms. The carbon atoms of such moiety may be part of an aliphatic, cycloaliphatic or aromatic moiety. In addition to the organic Lewis base functionality, such moieties may also contain other substituent atoms and/or groups, such as alkyl, aryl and halogen.

1) Chelating group or moiety as the metal binding domain: The term “chelating group” is art-recognized and refers to a molecule, often an organic one, and often a Lewis base, having two or more unshared electron pairs available for donation to a metal ion. It should be noted that chelating group or moiety is a small subgroup of metal binding domain (MBD) or Lewis base. The term chelating group may also be viewed as moiety with at least two Lewis bases capable of making at least two simultaneous coordinate bonds with a transition metal ion. For the purpose of the present invention, chelating group or moiety is a group or moiety pendant to the backbone capable of forming at least two coordinate bonds with metal ions. To be identified as chelating group or moiety, for the purpose of this invention, the moiety must be able to maintain its ability to form at least two coordinate bonding independent of its attachment to the backbone. Chelated metal ion is metal ion coordinated or coordinately bonded to least two electron pairs of the chelating group or moiety. The terms, “bidentate chelating”, “tridentate chelating group”, and “tetradentate chelating group” are art-recognized and refer to chelating groups having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible. For the purpose of the present specification the “chelating group” is the same as “chelating moiety” and is a single pendant or terminal portion of the molecule containing two or more electron pairs that can be donated to metal ions. The chelating moiety of the backbone is expected to maintain its chelating function even it is detached from the backbone while keeping the integrity of the backbone intact. Polylactic acid backbones without modification, polyamino acid backbones without modifications, and polysaccharides without modification do not have chelating groups or chelating moieties for the purpose of this specification.

Examples of metal binding domains which are chelating groups or act as chelating groups and can be chemically linked the backbone include:
1,4,7,10-tetraazacyclododecane-N,N′,N″,N′″-tetraacetic acid;
1,4,7,10-tetraaza-cyclododecane-N,N′,N″-triacetic acid;
1,4,7-tris(carboxymethyl)-10-(2′-hydroxypropyl)-1,4,7,10-tetraazocyclodecane;
1,4,7-triazacyclonane-N,N′,N″-triacetic acid;
1,4,8,11-tetraazacyclotetra-decane-N,N′,N″,N′″-tetraacetic acid;
1,2-diaminocyclohexane-N,N′,N″,N″′-tetraacetic acid;
bis(aminoethanethiol)carboxylic acid;
diethylenetriamine-pentaacetic acid (DTPA);
ethylenediamine-tetraacetic acid (EDTA);
ethyleneglycoltetraacetic acid (EGTA);
ethylene-bis(oxyethylene-nitrilo)tetraacetic acid;
ethylenedicysteine;
Imidodiacetic acid (IDA);
N-(hydroxyethyl)ethylenediaminetriacetic acid;
nitrilotriacetic acid (NTA);
nitrilodiacetic acid (NDA);
triethylenetetraamine-hexaacetic acid (TTHA);
Trimethyl-1,4,7-triazacyclononane (TACN);
2,3-Dimercaptopropanol (BAL);
Meso-2,3-Dimercaptosuccinic acid (DMSA);
Monoisoamyl meso-2,3-dimercaptosuccinic acid (Mi-ADMS);
Sodium 2,3-dimercapto-1-propanesulfonate (DMPS);
Cyclohexanediaminetetraacetic acid (CDTA);
D-Penicillamine (DPA);
N-acetylcysteine (NAC);
2-Mercaptopropionyl glycine (Tiopronin);
Sodium 4,5-dihydroxybenzene-1,3-disulfonate (Tiron);
Desferrioxamine (deferoxamine, DFO);
1,2-Dimethyl-3-hydroxypyridin-4-one (deferiprone, L1);
Triethylene tetramine (Trientine, Trien); or a
polypeptide having the formula: (AₓH_{y})ₚ, wherein A is any amino acid residue, H is histidine, x is an integer from 0-6; y is an integer from 1-6; and p is an integer from 2-6.

2) Non-chelating group as the Metal binding domain: Coordinate bonding that does not fit the definition of chelation as defined here is also part of the compositions of the present invention. This is when a metal ion has a single coordination bond with a single moiety. Similarly, metal binding us not chelation when a metal ion has a single coordination bond with a single moiety (first moiety) and there is a second coordination bond of the same metal with a second moiety far away (at least 15 atoms apart) from the first moiety. Because the Lewis basic groups function as the coordination site or sites for the metal cation, in certain embodiments, it may be preferable that the deformability of the electron shells of the Lewis basic groups and the metal cations be approximately similar. Such a relationship often results in a more stable coordination bond. For instance, sulfur groups may be desirable as the Lewis basic groups when the metal cation is a heavy metal. Some examples include the oligopeptides such as glutathione and cysteine, mercapto ethanol amine, dithiothreitol, amines and peptides containing sulfur and the like. Nitrogen containing groups may be employed as the Lewis basic groups when smaller metal ions are the metal. Alternatively, for those applications in which a less stable coordination bond is desired, it may be desirable that the deformability be dissimilar.

### C) Metal Ion:

The present invention contemplates the use of a variety of different metal ions. The metal ion may be selected from those that have usually two, three, four, five, six, seven or more coordination sites. A non-limiting list of metal ions for which the present invention may be employed (including exemplary and non-limiting oxidation states for them) includes Co³⁺, Cr³⁺, Hg²⁺, Pd²⁺, Pt²⁺, Pd⁴⁺, Pt⁴⁺, R³⁺, Ir³⁺, R³⁺, Co²⁺, Ni²⁺, Cu²⁺, Zn²⁺, Cd²⁺, Pb²⁺, Mn²⁺, Fe³⁺, Fe²⁺, Tc, Au³⁺, Au⁺, Ag⁺, Cu⁺, MoO₂²⁺, Ti³⁺, Ti⁴⁺, CH₃Hg⁺, and Y⁺³. In another embodiment, the non-limiting list of metal ions for which the present invention may be employed includes Zn²⁺, Ni²⁺, Co²⁺, Fe²⁺, Mn²⁺, and Cu²⁺. The metal ion contained in the metal bridge between the carrier and the active agent may have a therapeutic use itself, but it cannot serve as the active agent for the purpose of the present invention. In another embodiment of the invention the metal ion is any transition metal ion.

### D) Protective Side Chains:

Examples of Protective Chains include poly(ethylene glycol), which may be esterified by dicarboxylic acid to form a poly(ethylene glycol) monoester; methoxy poly(ethylene glycol) monoester (MPEG) or a copolymer of poly(ethylene glycol) and poly(propylene glycol) monoester preferably in a form of an ester with dicarboxylic acid giving the terminal of these polymer carboxyl group that can be used to covalently link them to the backbone. Another forms include poly(ethylene glycol)-carboxyl; methoxy poly(ethylene glycol)-carboxyl; poly(ethylene glycol)-carboxymethyl; methoxy poly(ethylene glycol)-carboxymethyl; poly(ethylene glycol) monoamine; methoxy poly(ethylene glycol) monoamine; poly(ethylene glycol) hydrazide; methoxy poly(ethylene glycol) hydrazide; and methoxy poly(ethylene glycol) imidazolide block-copolymer of poly (ethylene glycol). Overall molecular weight of a protective chain is preferentially larger than 300 Daltons but preferably not exceeding 15,000. A protective chain or chains are linked to the polymeric backbone by preferably a single linkage.

### E) Active Agents:

Active agents of the present invention are peptides, proteins, oligonucleotides, polynucleotides, peptidomimetics, deoxyribonucleic acids, ribonucleic acids, nucleic acid derivatives, oligosaccharides, polysaccharides, proteoglycan, or organic molecules. In some embodiments the organic molecules have a molecular weight of less than 2000 Daltons and includes their analogs, derivatives and fragments thereof comprising a metal binding domain capable of coordinate bonding with the metal ion, thus completing a bridge between an active agent and the chelating group covalently linked to the backbone of the carrier. An active agent may naturally contain at least one MBD, which may be used for binding to the carriers described above. A peptide, a protein, an oligonucleotide, a polynucleotide or a small molecular weight drug, therefore, can supplies an MBD naturally such that there is no need to provide one synthetically. An active agent may be loaded to the carrier of the present invention by mixing a carrier solution with an active agent such as metal binding peptide, protein, polynucleotide, oligonucleotide, or small drug with molecular weight of less than 2000 Daltons at temperature between 15 to 37 degrees Celsius. The loaded carrier can be lyophilized and reconstituted prior to use. The loaded carrier can be stored in a refrigerator (1 to 10 degree Celsius) prior to use. The an active agent of the present invention or active agents in general can be further modified to contain chelating group to enhance its binding to the carrier of the present invention. Chelating groups that can be used to modify an active agent includes all those listed in section above “chelating group as the metal binding domain”. The active agent of the present invention may or may not be recombinant product. The active agent of the present invention may be an active agent purified from organism that naturally produce active agent (peptide, protein, polynucleotide, oligonucleotide, or small drug with molecular weight of less than 2000 Daltons). The active agent of the present invention may be synthetically produced peptide, protein, polynucleotide, oligonucleotide, or small drug with molecular weight of less than 2000 Daltons. Examples of active agents are Growth factors (or hormones) such as Bone morphogenetic proteins (BMPs); Epidermal growth factor (EGF); Erythropoietin (EPO); Fibroblast growth factor (FGF); Granulocyte-colony stimulating factor (G-CSF); Granulocyte-macrophage colony stimulating factor (GM-CSF); Growth differentiation factor-9 (GDF9); Hepatocyte growth factor (HGF); Insulin-like growth factor (IGF); Myostatin (GDF-8); Nerve growth factor (NGF) and other neurotrophins; Platelet-derived growth factor (PDGF); Thrombopoietin (TPO); Transforming growth factor alpha(TGF-α); Transforming growth factor beta (TGF-β); Vascular endothelial growth factor (VEGF); nerve growth factor (NGF); brain-derived neurotrophic factor (BDNF); and Growth hormone (GH, somatropin). Further examples of active agents of the present invention are insulin, vasoactive intestinal peptide, glucagon like peptide, endostatin, angiostatin, trombospondin, interferons, blood clotting factors (VII, VIII), urokinase, interleukins (including IL-1, IL-1alpha, IL-1beta, IL-2, IL-3, IL-6, IL-7, and IL-8), interferons (alpha and gamma), tumor necrosis factor alpha (TNF-a), and any molecule able to bind metal either before or after attachment of a chelating group. All of the above examples of active agents are meant include their derivatives and analogs, including those modified by addition of metal binding domain or chelating group. Essentially active agent is any peptides, proteins, polynucleotides, oligonucleotides, or small drugs that coordinately binds metal ion. Example active agent that is a small drug that binds metal ion is doxorubicin and prostaglandins. Small molecular weight drugs (2000 Daltons or less) can be modified to contain chelating group that may or may not require removal by enzyme in the body to be active.

### ADVANTAGES OF THE INVENTION

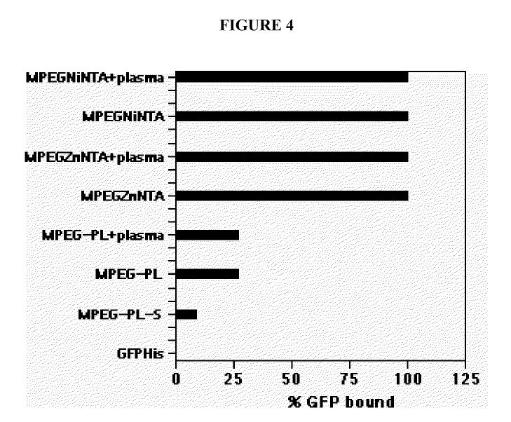

The carrier with coordinately bonded peptide, protein, or drug according to the present invention results in longer circulating in the body, more stable an active agent in the blood, and can be more conveniently administered (for example, quicker administrations such as through bolus instead of infusion, and less frequent administrations, e.g. once every few days instead of infusion or once a day). Often chronic administration of peptide, protein, or drug may result in immunogenic response. Carrier based formulations generally result in less immunogenicity than PEG based delivery systems so a peptide, protein, or drug is expected to be less immunogenic in compositions of the present inventions. “Direct PEGylation” of a peptide, protein, or drug is the direct bonding of a peptide, protein, or drug to PEG and can results in loss of activity. A peptide, protein, or drug coordinated with the chelated metal which is covalently linked to the backbone of the carrier with protective side chains, however, can result in a stable, long circulating alternative to PEGylation. The carrier of the present invention may act as a cryoprotectant and macromolecular stabilizer preserving a peptide, protein, or drug in solution as well as during the lyophilization and reconstitution process. It has been observed that carriers bearing chelated metal ion can bind biologically active peptides and proteins in the absence or presence of plasma proteins (FIG. 4). The subject compositions, and methods of making and using the same, may achieve a number of desirable results and features, one or more of which (if any) may be present in any particular embodiment of the present invention: 1) protecting active agent from the interaction with other macromolecules and cells; 2) decreasing undesirable immunogenicity of the carrier or active agent; 3) prolonging biological half-life of active agent in vivo (e.g. for decreasing glomerular filtration in kidneys, decreasing kidney and liver uptake, decreasing macrophage uptake etc); 4) stabilizing active agents by complexation with metal ion in the carrier. One advantage of the chelating moiety of the present invention is to afford reversible or labile binding with active agents which are capable of forming coordination bonds with metal ions (e.g. Zn²⁺, N²⁺, Co²⁺, Fe²⁺, Mn²⁺, or Cu²⁺). The coordinate bonding affords reversible dissociation of active agent from the carrier. It may be possible to affect the dissociation rate by choosing a different chelating group or moiety attached to the polymeric backbone or by including in the formulation a competitive ligands for the metal ion, such as imidazole or nitrilotriacetic acid (NTA).

### Sustained Release:

When the carrier of the present invention was formulated with an active agent (a peptide, a protein, a polynucleotide, an oligonucleotide, or a small drug), a release of active agent for extended period can be observed as evident from the sustain presence of active agent in the blood compared to active agent alone which can be determined by direct measurement of the active agent or by direct measurement of the effect of the active agent such as blood sugar level in case of insulin or glucagon like peptide. The association of carrier with the active agent is defined by specific dissociation constant (Kd) that can easily be determined by those skilled in the art. The release will be determined by the concentration of free active agent such that the when the free active agent concentration goes down (due to degradation or elimination by the body) and no longer satisfy Kd more active agent can be released to satisfy Kd. Kd is the product of concentration of free active agent and the concentration of chelated metal ions (not coordinately bonded to the active agent) divided by the concentration of the active agent coordinately bonded to the chelated metal ion. The advantage of the formulation is less frequent drug administration. The dosing can improve from continuous infusion to once a day or even once a week while providing a more constant level of active agent on the blood with less fluctuation compared to unformulated active agent. The frequency of bolus administration can vary according to the needs of the patient and can easily be determined by those skilled in the art.

### Dosages and Administration:

The dosage of active agent of the present invention will vary depending on the symptoms, age and body weight of the patient, the nature and severity of the disease, the route of administration, and the form of other supplemental drugs. Any of the subject formulations may be administered in a single dose or in divided doses. Dosages for the agent or a drug formulation of the present invention may be readily determined by techniques known to those skilled in the art.

In certain embodiments, the dosage of active agent formulation will generally be in the range of about 0.01 ng to about 1000 mg of an active agent per kg body weight, specifically in the range of about 1 ng to about 100 mg of an active agent per kg, and more specifically in the range of about 100 ng to about 20 mg of an active agent per kg. The more preferable dose range is about 100 ng to about 20 mg of an active agent per kg. The amount of an active agent relative to the weight of the carrier in a formulation may be in the range of about 1% to 1000% of the weight of the carrier. More preferably the amount of an active agent relative to the weight of the carrier in a formulation may be in the range of about 5% to 500% of the weight of the carrier. Even more preferably the amount of an active agent relative to the weight of the carrier in a formulation may be in the range of about 10% to 100% of the weight of the carrier.

An effective dose or amount, and any possible affects on the timing of administration of the formulation, may need to be identified in the present invention. This may be accomplished by routine experiment known to those skilled in the art, using one or more groups of animals (preferably at least 5 animals per group), or in human trials if appropriate. The effectiveness of an active agent formulation may be assessed by administering and assessing the effect of the administration by measuring one or more indices associated with the disease of interest, and comparing the post-treatment values of these indices to the values of the same indices prior to treatment.

The precise time of administration and amount of any particular compound that will yield the most effective treatment in a given patient will depend upon the activity, pharmacokinetics, and bioavailability of active agent, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

Treatment may be initiated with smaller dosages which are less than the optimum dose of the compound. Thereafter, the dosage may be increased by small increments until the optimum therapeutic effect is attained.

The combined use of an active agent formulation of the present invention with other therapeutic agents may reduce the required dosage for an active agent formulation. This is because the effect of other therapeutic agents may be complimentary to the effect of an active agent formulation. In such combined therapy, the different active agents may be delivered together or separately, and simultaneously or at different times within the day.

Toxicity and therapeutic efficacy of an active agent formulation of the present invention may be determined by standard pharmaceutical procedures in cell cultures or experimental animals, e.g., for determining the LD₅₀, ED₅₀, MIC, and/or MBC. Formulations that exhibit large therapeutic indices are preferred. Although formulations that exhibit toxic side effects may be used, care should be taken that a carrier-peptide, a carrier-protein, or a carrier-drug complex preferably accumulates at the desired site in order to reduce side effects.

The data obtained from the cell culture assays and animal studies may be used in formulating a range of dosage for use in humans. The dosage of active agent formulation must provide a range of circulating concentrations in the blood that is within the therapeutic range with little or no toxicity. The dosage may vary within this range depending upon the dosage form employed and the route of administration utilized. A dose of the formulation may be derived from animal models based on the dose that gives a circulating plasma concentration of active agent within the therapeutic range. Such information may be used to more accurately determine useful doses in humans.

The carrier with active agent of the present invention may be used for external administration in a form of ointment, paste, cream or gels and may further contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

The carrier with active agent of the present invention may be used for external administration in a form of powder or spray and may further contain excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

The carrier with active agent (a peptide, a protein, an oligonucleotide, a polynucleotide or a small molecular weight drug) of the present invention may be used for external administration in a form of aerosol. This is accomplished by preparing an aqueous aerosol, liposomal preparation or solid particles containing the composition of the present invention but not covalently bonded to the solid. A non-aqueous (e.g., fluorocarbon propellant) suspension could be used. Sonic nebulizers may be used because they minimize exposing the agent to shear, which may result in degradation of the compound. Ordinarily, an aqueous aerosol is made by formulating an aqueous solution or suspension of the formulation together with conventional pharmaceutically acceptable carriers and stabilizers. The excipients and stabilizers vary with the requirements of the particular compound, but typically include one or more of non-ionic surfactants (Tweens, Pluronics, or polyethylene glycol), innocuous proteins like serum albumin, sorbitan esters, oleic acid, lecithin, and amino acids such as glycine, buffers, salts, sugars or sugar alcohols. Aerosols generally are prepared from isotonic solutions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety

Pharmaceutical compositions of this invention suitable for parenteral administration comprise one or more components of a supplement in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous excipients which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXEMPLIFICATION

The invention is further illustrated by the following examples. The examples are provided for descriptive purposes only, and are not to be construed as limiting the scope or content of the invention to any backbone polymer, chelating group, metal ion, protective chain, and metal binding active agent in any way.

Examples of Compositions with a Polyamino Acid as the Polymeric Backbone

### Example 1

Synthesis of PLPEG (lot#20020101): Poly-L-lysine, hydrobromide (Sigma, Mw=48000, d.p. 200), 1 g was dissolved in 175 ml of 0.1 M Na₂CO₃, pH 8.7. An aliquot of this solution was removed for amino groups determination by TNBS titration (final concentration of NH₂-groups, 15 mM or 2.6 mmol total). Methoxy polyethylene glycol succinate (MPEGS9.6 g, 1.9 mmol) was dissolved in 25 ml of water, degassed, and N-hydroxy(sulfo)succinimide (500 mg, 2.3 mmol) was added, followed by 1 g, 5 mmol of EDC in 2 ml of water. This solution was incubated for 10 min at room temperature and added drop-wise to the solution of poly-1-lysine, final pH 7.7. The mixture was incubated for six hours. The product was purified using ultrafiltration on a cartridge with a cut-off of 100 kD (UFP-100 A/G Technology) to remove unconjugated MPEGS and other reactants.

### Example 2

Synthesis of PLPEGNTA: The product obtained as described in Example 1 (MPEGsuccinyl-poly-L-Lys (m.w. 340000)) was succinylated using 10-fold molar excess of succinic anhydride over the concentration of TNBS-reactive free aminogroups in the co-polymer in 0.5 M sodium carbonate pH 8.0, for 4 hours room temperature. Succinylated co-polymer (PLPEGSA) was purified using dialysis against water (lot#20020102).

100 mg Lyophilized PLPEGSA was dissolved in 2 ml water at 28 mol succinate/ml, treated with 30 mg ethyl-diaminopropyl carbodiimide (EDC) in the presence of 20 mg Sulfo-NHS for 10 min at room temperature. A solution of activated PLPEGSA was added to a 10 fold molar excess solution of N,N-Bis(carboxymethyl)-L-lysine Hydrate (BCMLys) in 1 ml sodium bicarbonate, pH 8.7. The final pH was adjusted to 7.6, incubated 24 hours at 4° C. The resultant product PLPEGNTA (lot#20020103) was purified using ultrafiltration on YM50 membrane (Amicon) by diluting to 100 ml and concentrating to 5 ml volume four times. A solution of PLPEGSA was used as a control in further experiments (lot#20020102).

### Example 3

Synthesis of PLPEGNTANi (lot#20020104): A solution of product PLPEGNTA was dialyzed against 1 L of 10 mM Ni acetate/20 mM citric acid, pH 6 for 24 hours at 4° C. and purified by dialyzing against 2 L water (2 changes). Binding of Ni was measured by spectrophotometry at 625 nm using Ni-citrate as a standard.

### Example 4

Synthesis of PLPEGNTAZn (lot#20020105): A solution of PLPEGNTA was dialyzed against 1 L of 10 mM Zn acetate/20 mM citric acid pH 6 for 24 hours at 4° C., and purified by dialyzing against 2 L water (2 changes). Binding of Zn was measured by using elemental analysis.

### Example 5

Synthesis of 40PLPEG5371DA (lot#20070927): One gram of 40 PL (Sigma P3995; lot# 085K5102; 1 g was found to contain 2.5 mmol NH₂ by TNBS assay according to Spadaro et. al. Anal Biochem, vol 96, p 317-321) was dissolved in 50 ml of 200 mM HEPES. 3.5 g of MPEGSuccinate (0.7 mmol; Mw=5 kDa; Sunbio; lot# CISA-005-07024) in 25 ml of 10 mM MES pH=4.7 was activated by adding 175 mg of NHSS (mw=217.14; 0.8 mmol, followed by 350 mg EDC (mw=191.71; 1.8 mmol). Activation was allowed to proceed for 20 minutes. The activated MPEGSuccinate was added to the 40 PL solution and allowed to react. After 2 hrs, additional 3.5 g of MPEGSuccinate was activated and added as above and stirred overnight. The next day amino groups were measured by TNBS and found to be 1.5 mmol indicating 40% saturation of amino group. The sample was lyophilized (13 g) without cleaning and stored at 4° C. for later use. The lyophilized sample was dissolved in 37 ml water, 2 g Succinic anhydride (SA, 20 mmol) was added, 200 ul TEA was added followed by titration (200 ul at a time) to pH 7.5-8.0 using 10M NaOH. The amino groups were measured by TNBS by taking 15 ul and diluting to 1 ml (67 fold; giving 0.2 mg/ml equivalent of original PL). No remaining amino group remaining was found. The resulting 40PLPEG537-succinate or 40PLPEG537SA was washed with 20 volumes of water using ultrafiltration cartridge with molecular weight cut off (MWCO) of 100 kDa (UFP-100-E-5A; GE Healthcare). The 40PLPEG537SA was dried and divided into two (2.95 g each). Iminodiacetic acid (IDA; 1.2 g; Mw=133; 9 mmol; Fisher Cat#AC20497) was prepared in 10 ml of 1M HEPES pH 7.35 in separate flask and pH was adjusted to pH 8.0 using 10 N NaOH. One portion of 40PLPEG537SA (2.95 g; 0.9 mmol carboxyl groups) was made up in 10 ml of 10 mM MES pH 4.7 and activated by adding 250 mg of NHSS (mw=217.14; 1.15 mmol, followed by 500 mg EDC (mw=191.71; 2.6 mmol). Activation of 40PLPEG537SA was allowed to proceed and after 20 minutes the activated 40PLPEG537SA was added to the IDA solution. After the reaction, the 40PLPEG5371DA product was washed with 25 volumes of water using an ultrafiltration cartridge with a molecular weight cut off (MWCO) of 100 kDa (UFP-100-E-5A; GE Healthcare). Total yield after drying was 2.43 g of 40PLPEG371DA (lot#20070927). The molecular diameter of this material was 19 nm as measured by GPC (column 78×30 cm; Tosoh G4000WXL; with PBS/15% Acetonitrile mobile phase flowing at 0.6 ml/min).

### Example 6

Synthesis of 40PLPEG535DADTPA (Lot#20071101A) and 40PLPEG535DADTPAIDA (20071101B): One g of 40 PL (P3995 Sigma lot# 085K5102) was dissolved in 50 ml of 200 mM HEPES. Amino groups were measured by the TNBS assay and were found to be 2.86 mmol NH2/g. Three grams of MPEGCM (MethoxyPolyEthyleneGlycol-CarboxyMethyl; 1 mmol; Mw=5 kDa; 9.0 mmol; Laysan Bio; lot#108-41; clear in solution) in 17.5 ml of 10 mM MES pH=4.7 and was activated by adding 150 mg of NHSS (mw=217.14; 0.7 mmol), followed by 300 mg EDC (mw=191.71; 1.57 mmol). Activation was allowed to proceed for 20 minutes. Total volume of MPEGCM solution at this stage was 18 ml. The activated MPEGCM was added to 40 PL solution and allowed to react. After 45 minutes, additional 3 g of MPEGCM was activated and added as above and allowed to react for 2 hrs Amino groups were measured by TNBS and found to be 103 uM giving 28% saturation. Size exclusion chromatography using a TosohG4000WXL column was performed (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.72 min on UV or 12.20 min on RI (18.4 nm). Another 1.5 g were activated and added to reach 35% amino group saturation based on the remaining amino groups as measured (91 uM in 94 ml or 1.82 mmol total) by TNBS. After addition of 1.5 g of MPEG retention time on UV becomes 11.60 min or 12.10 min on RI or 19 nm. Four grams of DTPA-dianhydride was added and the pH was adjusted continuously to maintain a pH between 7 to 8. After 4 hours, the total amino groups were measured by TNBS and found to be 0.0 uM. The reaction mixture containing 40PLPEG535DADTPA was washed with 20 volumes of water using ultrafiltration cartridge with molecular weight cut off (MWCO) of 100 kDa (UFP-100-E-5A; GE Healthcare) and lyophilized, giving 4.7 g (40PLPEG535DADTPA, lot#20071101A). Half (2.35 g) was saturated with iminodiacetic acid (IDA) as follows: IDA (3 gr) was made up to 10 ml of 1M HEPES, the pH adjusted to 7.5, and made up to 50 ml in 1M HEPES. Half of 40PLPEG535DADTPA was divided into 3 equal portions (1.3 mmol carboxyl each based on stoichiometry) and each (25 ml) made to pH 4.7 with 200 ul 1M MES, pH 4.7, the pH did not go down to 4.7 and therefore 20 ul of 6N HCl was added. This was activated by addition of 2 mmol NHSS (434 mg) and 4.5 mmol EDC (864 mg). After 20 minutes, the activated 40PLPEG535DADTPA was added to IDA above and repeated 2 more times and stirred for 2 hrs. The product (40PLPEG535DADTPAIDA) was washed with 20 volumes of water, filter-sterilized (0.2 um polysulfone filter, Nalgene, Rochester, N.Y.) and lyophilized giving 2.0 g (40PLPEG535DADTPAIDA, lot#20071101B).

### Example 7

Synthesis of 40PLPEG537DANTA (lot#20080124a): One g of 40 PL (Sigma P3995 lotnumber127K5101; 1 g was found to contain 2.84 mmol NH₂ as measured by TNBS) was dissolved in 50 ml of 200 mM HEPES. Five grams of MPEGCM (1 mmol; Mw=5 kDa; Sigma/Fisher/Fluka; Cat#70718; lot#64748/1) in 10 mM MES pH=4.7 in 25 ml of 60% ethanol (ethanol was needed to completely dissolve MPEG from Fisher/Fluka) was activated by adding 250 mg of NHS (mw=115.09; 2 mmol), followed by 500 mg EDC (mw=191.71; 1.8 mmol). Activation is allowed to proceed for 20 minutes (total volume is 29 ml). The activated MPEGCarboxyl was added to 40 PL solution and additional 6 ml of 1M HEPES added to keep the pH at about 7. The mixture was allowed to react overnight. The total volume in the morning was 82 ml and pH 7.04. The amino groups were measured by TNBS and found to be 1.74 mmol total indicating 39% saturation of amino groups. Succinic Anhydride (2 g) was added and pH adjusted to maintain at around 7.0 for 2 hours using 10 N NaOH (150 ul at a time approximately. 4 ml). After 2 hours, the amino groups were measured and no remaining amino groups were found. Sample was washed with 20 volume changes of water using a 100 kDa MWCO ultrafiltration cartridge (UFP-100-E-5A, GE-Amersham), filter-sterilized (0.2 um polysulfone filter, Nalgene, Rochester, N.Y.) and lyophilized (4.7 g).

One gram of NTA-amine (Nₐₗₚₕₐ, Nₐₗₚₕₐ, -Bis(carboxymethyl)-L-Lysine; Mw=262.26+aq, up to 2 mol water and 10% inorganic) or 3.8 mmol was dissolved in 21 ml of 1M HEPES, pH 7.35. Aliquot (10.5 ul) was taken and diluted to 10 ml for total amino group analysis and found to be 1.8 mmol.

40PLPEG538DASA (4.7 g; 1.7 mmol carboxyl) was dissolved in 33 ml of 10 mM MES, followed by addition of 500 mg NHS (mw=115.09; 4.3 mmol), followed by 2 gram EDC (mw=191.71; 10.4 mmol). During the reaction, pH was maintained below 5.5 (pH 5.0) by HCl while stirring After 12 minutes, the activated 40PLPEG538DASA was transferred to NTA-amine solution and pH was adjusted while stirring using 10 N NaOH to 7.0-7.1 (total reaction volume at this stage was 56 ml). After 3 days, a 30 ul aliquot was taken, diluted to 10 ml for amino group analysis, and amino groups were found to be 0.059 mmol total NH2. This was washed with 10 volume changes of water using a 100 kDa MWCO ultrafiltration cartridge (UFP-100-E-5A). The sample was lyophilized yielding 3.9 g (40PLPEG537DANTA, lot#20080124a). This material contains 15 nmol amino group/mg.

### Example 8

Synthesis of 40PLPEG537DANDA from NTA attached to the amino groups of Polylysine (lot#20080124b): One g of 40 PL (Sigma P3995 lotnumber127K5101; this time 1 g contains 2.62 mmol NH₂) was dissolved in 50 ml of 400 mM HEPES. Five g of MPEGCM (1 mmol; Mw=5 kDa; Sigma/Fisher/Fluka; Cat#70718; lot#64748/1) in 20 ml of 10 mM MES pH=4.7 with 60% ethanol (ethanol was needed to completely dissolve PEG from Sigma/Fisher/Fluka) was activated by adding 250 mg of NHS (mw=115.09; 2 mmol), followed by 500 mg EDC (mw=191.71; 1.8 mmol). Activation was allowed to proceed for 20 minutes (total volume is 20 ml). The activated MPEGCM was added to 40 PL solution (pH 7.45 before addition). The mixture was allowed to react for 4 hrs, the total volume was 71 ml and pH was 7.14 at the end. The amino group measurement before (2.62 mmol) and after (1.64 mmol) MPEG addition indicated that the PEG saturation of amino groups was 37%. Size exclusion chromatography using a TosohG4000WXL column was performed (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.5 min (or approximately 16 nm in diameter). This is the 40PLPEG537DA solution.

NTA (MW=191; 1 g or 5.2 mmol) was neutralized in water with 1 ml of 10N NaOH and buffered with 20 mM MES at pH 4.7 (total volume is 10 ml). This was activated (in 10 ml of 20 mM MES) with 1 g (5.2 mmol) EDC in the presence of 345 mg NHS (mw=115.09; 3 mmol). After 20 minutes this was added to 40PLPEG537DA. The initial pH of 40PLPEG537DA solution was pH 7.14 but goes down to 6.9 after addition of activated NTA. This was adjusted to 7.25 with 300 ul of 10N NaOH and allowed to react overnight (total volume is 82 ml). The next day, the amino groups were measured and found to be only slightly decreased. The pH was lowered to 4.7 using HCl, 2 g EDC was added, and after 20 minutes the pH was raised to 7.0 using 10N NaOH. After 2 hours, the process was repeated and after additional 2 hours the total amino groups were measured and found to be 0.03 mmol which is compared to 1.63 mmol original amino groups before the reaction. Sample was washed with 20 volume changes of water using a 100 kDa MWCO ultrafiltration cartridge (UFP-100-E-5A; GE-Amersham), filter-sterilized (0.2 um polysulfone filter; Nalgene, Rochester, N.Y.) and lyophilized giving 4.1 g of 40PLPEG537DANDA.

### Example 9

Synthesis of 20PLPEG570DANTAZn (Lot#20080326): a) Two g of 20PL (Q4926 SAFC lot# 018K7775; DP=126; 2 g has 4.76 mmol NH2) was dissolved in 25 ml of 1 M HEPES Amino groups were measured by TNBS assay and found to be 4.76 mmol NH2/g. b) 14 g of MPEGCM (2.8 mmol; SAFC lot#1372618; orange-yellow in solution) in 52 ml of 50% ethanol with 10 mM MES pH4.7 was activated by adding 700 mg of NHS (mw=115.14; 6.09 mmol), followed by 1.4 g EDC (mw=191.71; 7.30 mmol). Activation is allowed to proceed for 20 minutes. c) The activated MPEGCM was added to 20PL solution and allowed to react 2 hours. When the amino groups were measured, only 46% saturation was found, thus additional MPEGCM was activated and added (1.2 g) and incubated overnight. Amino group analysis indicated 71% PEG saturation. d) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.75 min by refractive index or approximately 15 nm molecular diameter. e) Succinic Anhydride (5 g; 50 mmol) added and slowly titrated with 10 N NaOH to pH 7.0 while stirring. After 4 hours the amino groups were measured and found to be 0 uM. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.5 min by UV 220 and 12.6 min by refractive index or approximately 16 nm molecular diameter. The reaction mixture was washed using a 100 kDa MWCO filter cartridge (UFP-100-E-5A; GE-Amersham), filter-sterilized (0.2 um filter polysulfone filter; Nalgene, Rochester, N.Y.), and lyophilized (40PLPEG570SA; 9.9 g; contains 1.36 mmol carboxyl by stoicheometry). f) Two gram of NTA-amine (Nalpha,Nalpha,-Bis(carboxymethyl)-L-Lysine; Mw=262.26+aq, up to 2 mol water/mol and 10% by weight inorganic) or 7.6 mmol was dissolved in 40 ml of 1M HEPES. Actual amino group measurement indicated 4.28 mmol NTA-amine g) 40PLPEG570SA (9.9 g; 1.36 nmol Carboxyl) was dissolved in 72 ml of 20 mM MES, and 500 mg NHS (mw=115.09; 4.3 mmol but with water so perhaps 3) was added, followed by 2 gram EDC (mw=191.71; 10.4 mmol). The pH went up slowly during the 20 minute reaction but the pH was maintained below 5.5 by HCl. This solution was added to NTA-amine solution. After 2 hours, amino group analysis, indicated that total NTA-amine was down to 3.08 mmol indicating that 1.2 mmol NTA was incorporated into the carrier. h) The reaction mixture was washed with 10 volumes of water using a 100 kDa MWCO filter cartridge (UFP-100-E-5A; GE-Amersham), filter-sterilized (0.2 um polysulfone filter; Nalgene, Rochester, N.Y.), and lyophilized (9.0 g; 20PLPEG570DANTA; Lot#20080326). Size exclusion chromatography using a TosohG4000WXL column was performed (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.36 min by refractive index or 12.1 min by UV220 nm or approximately 17.5 nm molecular diameter.

### Example 10

Synthesis of 20PLPEG550DADTPANTA (Lot#20080411): a) 1 mL or 0.4 g equivalent of 20PL (Q4926 SAFC lot# 018K7775; DP=126; 0.4 g was found to contain 0.895 mmol NH2 by TNBS) was dissolved in 5 ml of 1 M HEPES. This is the 20PL solution. b) In a separate container, 2.5 g MPEG was activated in 20 mM MES pH=4.7 (35 ml) by adding 125 mg of NHS (mw=115.14; 1.09 mmol) and 500 mg EDC (mw=191.71; 2.60 mmol) while stirring. Activation was allowed to proceed for 20 minutes and the activated MPEGCM was added directly to 20PL solution in step a. The pH of the reaction mixture was adjusted to pH 7.1 slowly with 10N NaOH one drop at a time, and allowed to react for overnight Amino group analysis by TNBS showed 0.464 mmol amino groups remained, indicating 50% PEG saturation. This is the 20PLPEG550DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min in refractive index detector or approximately 14.4 nm molecular diameter. d) Diethylenetriaminepentaacetic acid dianhydride (1 gram; FW=357.3; 2.80 mmol) was added and slowly titrated with 10 N NaOH to pH 7.1 and stirred for 2 hours. After 2 hours, amino group measurement by TNBS indicated 0% amino group remains. e) The pH of the solution was adjusted to 7.5 using 10N NaOH to facilitate washing as crystals of un-reacted DTPA remains. The solution was concentrated to 100 ml and washed with 15 changes of water using a 100,000 MWCO ultrafiltration cartridge (UFP-100-E-5A; GE-Amersham) and lyophilized. Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.7 min in a refractive index detector or approximately 15.04 nm molecular. f) 2 gram of NTA-amine (Nalpha,Nalpha, -Bis(carboxymethyl)-L-Lysine; Mw=262.26+50% impurity, up to 2 mol water and 10% inorganic) or ˜4 mmol amino groups was dissolved in 10 ml of 1M HEPES. Amino group analysis by TNBS indicated that the NTA-amine solution contained 3.4 mmol amino groups. g) 20PLPEG550DADTPA (0.70 mmol carboxyl) was dissolved in 10 ml of 20 mM MES, 140 mg NHS (mw=115.09; 1.2 mmol) was added, followed by 560 mg EDC (mw=191.71; 2.9 mmol). The pH went up slowly but was maintained below 5.5 by HCl. This solution was added to NTA solution and the pH was adjusted to pH 7.1 with 10N NaOH. After 2 hours, amino group analysis showed a total of 3.2 mmol amino groups remains, indicating that 0.2 mmol of NTA-amine was incorporated to 0.8 mg carrier. h) The solution was concentrated to 100 ml and washed with 15 changes of water using a 100,000 MWCO ultrafiltration cartridge (UFP-100-E-5A; GE-Amersham), filter-sterilized (0.2 um polysulfone filter, Nalgene, Rochester, N.Y.) and lyophilized yielding 0.5 g (20PLPEG550DADTPANTA; Lot#20080411). Size exclusion chromatography using a TosohG4000WXL column was performed (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.7 min in a refractive index detector showing approximately 15.04 nm molecular diameter.

### Example 11

Synthesis of 20PLPEG1055DANTA (Lot#20080416): a) 5 mL or 2 g equivalent of 20PL (Q4926 SAFC lot# 018K7775; DP=126; 2 g was found to contain 4.60 mmol NH2 by TNBS) was dissolved in 25 ml of 1 M HEPES. This is the 20PL solution. b) In a separate container, 20 g of MPEGCM (Mw=10 kDa; 2.0 mmol; SunBright; ME-100HS; lot#M62503; clear solution) was dissolved in 60 ml of 80% ethanol with 20 mM MES pH=4.7 (1200 ul of 1M MES added to 60 ml), 500 mg of NHS (mw=115.14; 4.35 mmol) was added, once dissolved 2.0 g EDC (mw=191.71; 10.43 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. Amino group analysis showed 1.92 mmol remains indicating 58% MPEG saturation. This is the 20PLPEG1055DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min (or approximately 22.2 nm molecular diameter) and also showing 95% incorporation of MPEG. d) Succinic Anhydride (2 g; 20 mmol) was added followed by 200 uL TEA. The reaction was slowly titrated with 10 N NaOH to pH 7.1 and stirred for 4 hours. The amino groups was measured and found to be 0 umol. Size exclusion chromatography using a TosohG4000WXL column was performed (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or 21 nm molecular diameter. The reaction mixture was washed with 15 volumes of water using a 100,000 MWCO ultrafiltration cartridge (UFP-100-E-5A; GE-Amersham) and lyophilized (13.1 g). e) 2 gram of NTA-amine (Nalpha,Nalpha, -Bis(carboxymethyl)-L-Lysine; Mw=262.26+50% impurity, up to 2 mol water and 10% inorganic) or ˜4 mmol was dissolved in 10 ml of 1M HEPES. Twenty ml of 0.5M ZnCl was added to the NTA-amine and adjusted to pH7.1 with 10N NaOH. The solution was centrifuge and supernatant was collected and total amino groups were determined by TNBS. The TNBS measurement indicated total amino group of 4.80 mmol f) 20PLPEG1055DASA (3.1 g or 0.40 mmol carboxyl) was dissolved in 15 ml of 20 mM MES, 115 mg NHS (Mw=115.09; 1 mmol) was added, followed by 500 mg EDC (mw=191.71; 2.6 mmol). The pH went up slowly but was maintained at 4.7 by HCl. After 20 minutes, 20PLPEG1055DASA solution was added to NTA-amine supernatant and pH was adjusted to 7.1 using 10N NaOH. After 2 hours, total amino groups were measured by TNBS and found to be 4.0 mmol indicating 0.80 mmol NTA-amine was incorporated to 3.1 g carrier. g) To remove Zinc, 2 g of NTA (Nitrilotriacetic acid; Mw=191.14) or 10 mmol was added to solution in f and adjusted to pH 7.0 with 10N NaOH, followed by 10 ml of Imidazole (5M) and pH goes up to 8. h) The 20PLPEG1055DA-NTA was washed with 10 volumes of water using a 100,000 MWCO ultrafiltration cartridge (UFP-100-E-5A; GE-Amersham). Sample 20PLPEG1055DANTA was filter-sterilized (0.2 um polysulfone filter; Nalgene, Rochester, N.Y.) and lyophilized (2.60 g; 20PLPEG1055DANTA; Lot#20080416). Ten mg/ml of 20PLPEG1055DANTA was analyzed by size exclusion chromatography. Size exclusion chromatography using a TosohG4000WXL column was performed (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min using UV detector or 11.9 min using refractive index detector (or approximately a 20.6 nm molecular diameter). 1 mg/ml was analyzed by TNBS and contain 0+/−5 uM NH2 or 0 nmol/mg

### Example 12

Synthesis of 20PLPEG550DATACNZn (Lot#20080606): a) Fifteen ml (6 gm of 20PL; Q4926 SAFC lot# 018K7775; DP=126; 2 gm has 4.72 mmol NH2 as measured by TNBS) was added to 135 ml of 1 M HEPES to make the 20PL solution. b) In a separate container, 45 g of MPEGCM (Mw=5000; 9.0 mmol; Laysan Bio; lot#108-41; clean in solution) was dissolved in 150 ml of 80% ethanol with 10 mM MES pH=4.7 (1500 ul of 1M MES added to 150 ml) and with 2250 mg of NHS (mw=115.14; 19.6 mmol), once dissolved 4500 mg EDC was added (mw=191.71; 23.5 mmol) while stirring. Activation was allowed to proceed for 20 minutes and the activated MPEG-CM was added directly to the 20PL solution in a. The pH was adjusted to pH 7.1 slowly with 10N NaOH one drop at a time, and allowed to react for 2 hours. Aliquots (exactly 10 ul per 100 ml solution or 11.5 ul in this case 115 ml) were taken and diluted to 4 ml water for amino group analysis. It was found to be 2.16 mmol total, indicating 54% PEG saturation. This is the 20PLPEG550DA solution. c) This was confirmed by size exclusion chromatography using TosohG4000WXL (0.79×30 cm) and found to have a retention time of 12.8 min (approximately 14.4 nm) and also showing 95% incorporation of PEG. d) Succinic Anhydride (6 grams; 20 mmol) was added and followed by 600 uL TEA. The reaction was slowly titrated with 10 N NaOH to pH 7.1 and stirred for 4 hours and amino groups was measured and found to be 0 uM. Size exclusion chromatography using TosohG4000WXL (0.79×30 cm) showed a retention time of 12.3 min (approximately 17.6 nm). e) The reaction mixture containing 20PLPEG550DASA was concentrated to 400 ml and washed with 15 changes of water in a 100,000 MWCO ultrafiltration cartridge (UFP-100-E-5A). f) The sample was filtered sterilized using 0.2 um filter (polysulfone) and lyophilized giving a total of 31 grams (Lot#20080523). g) Eight g of 20PLPEG555DASA (theoretically contains 2 mmol Carboxyl) was activated by dissolving in ethanol and adding 460 mg NHS (Mw=115; 4 mmol), followed by 2 g EDC (mw=191.71; 10 mmol) (43 ml final volume). The activation was allowed to proceed for 20 minutes and the activated 20PLPEG555DASA was added directly to solution Triazacyclononane (3 gram; TACN; Mw=238.59; 12.5 mmol) in 50 ml (25 ml 1M HEPES and 25 ml ethanol) and allowed to react for 2 hours. The reaction mixture was concentrated to 100 ml and washed with 15 changes with 80% ethanol in 100,000 MWCO ultrafiltration cartridge (UFP-100-E-5A), followed by 5 volume wash with water and lyophilized giving a total of 7 g.

### Example 13

Synthesis of 35PEPEG1035DANTA: This composition will have polyglutamic acid (Sigma Chem. Co. St Luis Mo. Cat#P4033, Mw=15-50 kDa; or Fisher Chem Co. Pittsburg, Pa., Cat#ICN15191891, Mw=15-50 kDa) as the polymeric backbone with about 35% of the carboxyl groups occupied with 10 kDa MPEGAM (Mw=10 kDa MPEG with an amino group at the terminal; Sunbio, Orinda, Calif.; Cat#P1AM-10) and the remaining carboxyl groups occupied with nitrilodiacetic acid. The starting carboxyl group is measured according to the protocol by Kobayahi and Chiba (Analytical biochemistry 1994, vol 219, p 189-194). To synthesize 35PEPEG1035DANTA, 1.0 g of polyglutamic acid (Sigma Chem. Co. St Luis Mo. Cat#P4033; Mw=15-50 kDa, with Xmmol carboxyl group/gram) is dissolved in 25 ml of 20 mM MES (2-(N-morpholino)ethanesulfonic acid, Pierce, Rockford, Ill.) buffer pH 4.7 to make PE solution. 0.35×mmol of MPEGAM (Mw=10 kDa MPEG with amino group at the terminal; Sunbio, Orinda, Calif.; Cat#P1AM-10) in 20 ml of 80% ethanol is added to the PE solution to make a 35PEPEG solution. To the 35PEPEG solution, 1×mmol of NHS (Mw=115.14) and 1×mmol EDC (mw=191.71) are added while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If pH is below 7.1 the pH is adjusted with 10N NaOH one drop at a time to reach 7.1. The reaction is allowed to proceed for 2 hours and the remaining amino groups of the MPEGAM are measured by TNBS. It is expected that the amino groups measurement is expected to be none indicating that all 0.35×mmol amino group is used up and conjugated to the polyglutamic acid. If there are remaining amino group, the pH is adjusted to 5 with 6N HCl and Xmmol EDC (Mw=191.71) is added, and after 20 minutes the pH is adjusted back to 7.1 and allowed to react overnight. An aliquot of the resulting 35PEPEG1035 solution is taken and the hydrodynamic diameter is determined by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with hydrodynamic diameter of approximately 16-24 nm. The pH is adjusted down to 5 with 6N HCl and 1.5×mmol EDC (Mw=191.71) is added and activated for 20 minutes. After 20 minutes this activated 40PEPEG1035 solution is added to 10×mmol of Nalpha,Nalpha-biscarboxymethyl-lysine (Mw=262Da) in 25 ml of 1M HEPES buffer at pH 7.5 and allowed to react overnight. The reaction mixture is concentrated to 100 ml and washed with 15 changes of water in a 100 kDa-MWCO ultrafiltration cartridge (UFP-100-E-5A). The sample is filter-sterilized using (0.2 um polysulfone filter, Nalgene, Rochester, N.Y.) and lyophilize. A 10 mg/ml solution of resulting 35PGPEG1035DANTA is made and the hydrodynamic diameter determined by size exclusion chromatography using a TosohG4000WXL column (0.79×30 cm) and phosphate buffered saline as elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with a hydrodynamic diameter of approximately 19-28 nm.

### Example 14

Synthesis of 10PDPEG1035DANTA: This composition will have a polyaspartic acid (Sigma Chem. Co. St Luis Mo. Cat#P5387, Mw=5-15 kDa) as the polymeric backbone with about 35% of the carboxyl groups occupied with 10 kDa MPEGAM (Mw=10 kDa MPEG with amino group at the terminal; Sunbio, Orinda, Calif.; Cat#P1AM-10) and the remaining carboxyl groups occupied with nitrilodiacetic acid. The starting carboxyl group (Xmmol) of 1.0 g poly-glutamic acid is measured according to the protocol by Kobayahi and Chiba (Analytical biochemistry 1994, vol 219, p 189-194). To synthesize 10PDPEG1035DANTA, 1.0 g of polyaspartic acid (Sigma Chem. Co. St Luis Mo. Cat#P4033; Mw=15-50 kDa, with Xmmolcarboxyl group/gram) is dissolved in 25 ml of 20 mM MES (2-(N-morpholino)ethanesulfonic acid, Pierce, Rockford, Ill.) buffer pH 4.7 to make the PD solution. 0.35×mmol of MPEGAM (Mw=10 kDa MPEG with amino group at the terminal; Sunbio, Orinda, Calif.; Cat#P1AM-10) is dissolved in 20 ml of 80% ethanol and added to the PD solution to make PDPEG solution. To the PDPEG solution, Xmmol NHS (Mw=115.14) and Xmmol EDC (mw=191.71) are added while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If pH is below 7.1 the pH is adjusted with 10N NaOH one drop at a time to reach 7.1. The reaction is allowed to proceed for 2 hours and the remaining amino groups of the MPEGAM are measured by the TNBS assay and is expected be 0, indicating that all 0.35×mmol amino group is used up and conjugated to the polyaspartic acid. If there are remaining amino group, the pH is adjusted to 5 with 6N HCl and add Xmmol EDC (mw=191.71), after 20 minutes adjusted back to a pH to 7.1 and allowed to react overnight. An aliquot of the resulting 10PDPEG1035 solution is used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected be consistent with a hydrodynamic diameter of approximately 12-18 nm. The pH is adjusted down to 5 with 6N HCl and 1.5×mmol EDC (mw=191.71) is added and activated for 20 minutes. After 20 minutes this activated 10PDPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The sample is filter-sterilized (using a 0.2 um polysulfone filter, Nalgene, Rochester, N.Y.) and lyophilized. A 10 mg/ml solution is made of the resulting 10PDPEG1035DANTA and the hydrodynamic diameter determined by size exclusion chromatography using TosohG4000WXL column (0.79×30 cm) and phosphate buffered saline as elution solvent (PBS; 11.9 mM phosphate, 137 mM NaCl, 2.7 mMKPO₄, pH 7.4) containing 15% Acetonitrile at a flow rate of 0.6 ml/min. The retention time is measured and the hydrodynamic diameter is expected to be approximately 14-20 nm.

### Example 15

Synthesis of 10PSPEG1035DANTA: This composition will have polyserine (Sigma Chem. Co. St Luis Mo. Cat#P5857, Mw=5-15 kDa) as the polymeric backbone with 35% of the hydroxyl groups occupied with 10 kDa MPEGAM (Mw=10 kDa MPEG with amino group at the terminal; Sunbio, Orinda, Calif.; Cat#P1AM-10) and the remaining hydroxyl group occupied with nitrilodiacetic acid. To prepare this composition all hydroxyl groups are converted to carboxyl groups using succinic anhydride. Briefly, 1 g of polyserine is dissolved in 25 ml of dioxane and 5.2 gram of succinicanhydride (five fold molar excess over total theoretical hydroxyl group) is added to this and 900 mg of N,N′-dimethylaminopryridine is added as a catalyst and the mixture is incubated at 60° C. for 3 hours. The dioxane is removed by rotary evaporation at 40° C., and the solid is dissolved in water, neutralized with NaOH and washed with 15 changes of water in a 3 kDa-MWCO ultrafiltration cartridge (UFP-3-E-5A). The resulting product (PS) is filter-sterilized (using a 0.2 um polysulfone filter, Nalgene, Rochester, N.Y.) and lyophilized. The starting carboxyl group is measured (Xmmol) in 1.0 g PS according to the protocol by Kobayahi and Chiba (Analytical biochemistry 1994, vol 219, p 189-194). To synthesize 10PSPEG1035DANTA, 1.0 g of PS (with Xmmol Carboxyl group) is dissolved in 25 ml of 20 mM MES (2-(N-morpholino)ethanesulfonic acid, Pierce, Rockford, Ill.) buffer pH 4.7 (PS solution). 0.35×mmol of MPEGAM (Mw=10 kDa MPEG with amino group at the terminal; Sunbio, Orinda, Calif.; Cat#P1AM-10) is dissolved in 20 ml of 80% ethanol and added to the PS solution to make a PSPEG solution. To the PSPEG solution, Xmmol of NHS (Mw=115.14) and Xmmol EDC (Mw=191.71; 4 mmol) are added to the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If pH is below 7.1 the pH is adjusted with 10N NaOH one drop at a time to reach 7.1. The reaction is allowed to proceed for 2 hours and the remaining amino groups of the MPEGAM are measured by TNBS and is expected be 0 indicating that all 0.35×mmol amino group is used up and conjugated to the PS. If there are remaining amino group, the pH is adjusted to 5 with 6N HCl and Xmmol EDC (mw=191.71) added, after 20 minutes the pH is adjusted back to 7.1 and allowed to react overnight. An aliquot of the resulting 10PSPEG1035 solution is taken and used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be with hydrodynamic diameter of approximately 13-18 nm. The pH is adjusted down to 5 with 6N HCl and 1.5×mmol EDC (Mw=191.71) is added and activated for 20 minutes. After 20 minutes this activated 10PSPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The sample is filter-sterilized using (0.2 um polysulfone filter, Nalgene, Rochester, N.Y.) and lyophilized. A 10 mg/ml solution of the resulting 10PSPEG1035DANTA is made and the hydrodynamic diameter is determined by size exclusion chromatography using a TosohG4000WXL column (0.79×30 cm) and phosphate buffered saline as an elution solvent (PBS; 11.9 mM phosphate, 137 mM NaCl, 2.7 mMKPO₄, pH 7.4) containing 15% Acetonitrile at a flow rate of 0.6 ml/min. The retention time is measured and the hydrodynamic diameter is expected to be 14-19 nm.

### Example 16

Synthesis of 10PTPEG1035DANTA: This composition will have polythreonine (Sigma Chem. Co. St Luis Mo. Cat#P8077, Mw=5-15 kDa) as the polymeric backbone with 35% of the hydroxyl groups occupied with 10 kDa MPEGAM (Mw=10 kDa MPEG with amino groups at the terminal; Sunbio, Orinda, Calif.; Cat#P1AM-10) and the remaining hydroxyl groups occupied with nitrilodiacetic acid. To prepare this composition all hydroxyl groups will be converted to carboxyl group using succinic anhydride. Briefly, 1 g of polythreonine is dissolved in 25 ml of dioxane and 4.5 gram of succinicanhydride added (five fold molar excess over theoretical hydroxyl group in 1 gram of polythreonine) and 900 mg of N,N′-dimethylaminopryridine added as a catalyst and the mixture incubated at 60° C. for 3 hours. The dioxane is removed by rotary evaporation at 40° C., the solid dissolved in water, neutralized with NaOH and washed with 15 changes of water in a 3 kDa-MWCO ultrafiltration cartridge (UFP-3-E-5A). The resulting product (PT) is filter-sterilized using a 0.2 um polysulfone filter (Nalgene, Rochester, N.Y.) and lyophilized to obtain PT. The starting carboxyl group is measured (Xmmol) in 1.0 g PT according to the protocol by Kobayahi and Chiba (Analytical biochemistry 1994, vol 219, p 189-194). To synthesize 10PTPEG1035DANTA, 1.0 g of PT (with Xmmol carboxyl group) is dissolved in 25 ml of 20 mM MES (2-(N-morpholino)ethanesulfonic acid, Pierce, Rockford, Ill.) buffer pH 4.7 (PS solution). 0.35×mmol of MPEGAM (Mw=10 kDa MPEG with amino group at the terminal; Sunbio, Orinda, Calif.; Cat#P1AM-10) is dissolved in 20 ml of 80% ethanol and added to the PS solution to make a PTPEG solution. To the PTPEG solution, Xmmol of NHS (Mw=115.14) and Xmmol EDC (Mw=191.71) are added to the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If pH is below 7.1 the pH is adjusted with 10N NaOH one drop at a time to reach 7.1. The reaction is allowed to proceed for 2 hours and the remaining amino groups of the MPEGAM are measured by the TNBS assay and is expected be none, indicating that all 0.35×mmol amino group is used up and conjugated to the PT. If there are remaining amino groups, the pH is adjusted to 5 with 6N HCl and Xmmol EDC (Mw=191.71) is added, and after 20 minutes adjusted back the pH to 7.1 and allowed to react overnight. An aliquot of the resulting 10PTPEG1035 solution is used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as an elution solvent at a flow rate of 0.6 ml/min. The retention time is expected to be consistent with the hydrodynamic diameter of approximately 12-18 nm. The pH is adjusted to down to 5 with 6N HCl and 1.5×mmol EDC is added (Mw=191.71) and activated for 20 minutes. After 20 minutes this activated 10PTPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The 10PTPEG1035DANTA product is filter-sterilized (using a 0.2 um polysulfone filter, Nalgene, Rochester, N.Y.) and lyophilized. The 10 mg/ml solution of resulting 10PTPEG1035DANTA is used to determine the hydrodynamic diameter by size exclusion chromatography using a TosohG4000WXL column (0.79×30 cm) and phosphate buffered saline as an elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with a hydrodynamic diameter of approximately 14-20 nm.

### Example 17

Synthesis of 20PYPEG1035DANTA: This composition has polytyrosine (Sigma Chem. Co. St Luis Mo. Cat#P1800, Mw=10-40 kDa) as the polymeric backbone with 35% of the hydroxyl groups occupied with 10 kDa MPEGAM (Mw=10 kDa MPEG with amino groups at the terminal; Sunbio, Orinda, Calif.; Cat#P1AM-10) and the remaining hydroxyl groups occupied with nitrilodiacetic acid. To prepare this composition all hydroxyl groups will be converted to carboxyl groups using succinic anhydride. Briefly, 1 g of polytyrosine is dissolved in 25 ml of dioxane and 2.9 gram of succinicanhydride is added (five fold molar excess over theoretical hydroxyl group in 1 gram of polythreonine) and 900 mg of N,N′-dimethylaminopryridine is added as a catalyst and the mixture incubated at 60° C. for 3 hours. The dioxane is removed by rotary evaporation at 40° C., the solid dissolved in water, neutralized with NaOH and washed with 15 changes of water in a 3 kDa-MWCO ultrafiltration cartridge (UFP-3-E-5A). The resulting product (PY) is filter-sterilized using a 0.2 um polysulfone filter (Nalgene, Rochester, N.Y.) and lyophilized to obtain PY. The starting carboxyl group is measured (Xmmol) in 1.0 g PY according to the protocol by Kobayahi and Chiba (Analytical biochemistry 1994, vol 219, p 189-194). To synthesize 20PYPEG1035DANTA, 1.0 g of PY (with Xmmol carboxyl group) is dissolved in 25 ml of 20 mM MES (2-(N-morpholino)ethanesulfonic acid, Pierce, Rockford, Ill.) buffer pH 4.7 (PS solution). 0.35×mmol of MPEGAM is dissolved (Mw=10 kDa MPEG with amino groups at the terminal; Sunbio, Orinda, Calif.; Cat#P1AM-10) in 20 ml of 80% ethanol and added to the PY solution to make a PYPEG solution. To the PYPEG solution, Xmmol of NHS (Mw=115.14) and Xmmol EDC (mw=191.71) are added to the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If the pH is below 7.1 the pH is adjusted with 10N NaOH, one drop at a time to reach 7.1. The reaction is allowed to proceed for 2 hours and the remaining amino groups of the MPEGAM are measured by TNBS and it is expected to be none indicating that all 0.35×mmol amino group is used up and conjugated to the PY. If there are remaining amino groups, the pH is adjusted to 5 with 6N HCl and Xmmol EDC added (mw=191.71), and after 20 minutes the pH is adjusted back to 7.1 and allowed to react overnight. An aliquot of the resulting 20PYPYG1035 solution is used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with hydrodynamic diameter of approximately 13-18 nm. The pH is adjusted down to 5 with 6N HCl and 1.5×mmol EDC (mw=191.71) is added and activated for 20 minutes. After 20 minutes this activated 20PYPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The sample is filter-sterilized using a 0.2 um polysulfone filter (Nalgene, Rochester, N.Y.) and lyophilized. A 10 mg/ml solution of resulting 20PYPEG1035DANTA is made and the hydrodynamic diameter is determined by size exclusion chromatography using a TosohG4000WXL column (0.79×30 cm) and phosphate buffered saline as an elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with a hydrodynamic diameter of approximately 14-20 nm.

### Example 18

Synthesis of 20PCPEG1035DAEDTA: This composition has polycysteine (Sigma Chem. Co. St Luis Mo. Cat#P1800, Mw=10-40 kDa) as the polymeric backbone with 35% of the thiol groups occupied with 10 kDa MPEGCM (Mw=10 kDa; SunBright; ME-100HS; lot#M62503; clean in solution) and the remaining thiol groups occupied with ethyenediaminetetraacetic acid (EDTA). To prepare this composition all thiol groups will be converted to amino groups using Aminoethyl-8 (N-(Iodoethyl) Trifluoroacetamide (Pierce, Rockford, Ill., Cat#23010). Briefly, 1 g of polycysteine is dissolved in 25 ml of 20 mM tricine buffer at pH 8.5 and 4.7 gram of Aminoethyl-8 (N-(Iodoethyl) Trifluoroacetamide is added (2 fold molar excess over theoretical thiol group in 1 gram of polycysteine). The mixture is incubated at room temperature for 3 hours, and washed with 15 changes of water in a 3 kDa-MWCO ultrafiltration cartridge (UFP-3-E-5A). The resulting product (PS) is filter-sterilized using a 0.2 um polysulfone filter (Nalgene, Rochester, N.Y.) and lyophilized to obtain PC. The starting amino group is measured (Xmmol) in 1.0 g PC using the TNBS assay as above. To synthesize 20PCPEG1035DAEDTA, 1.0 g of PC (with Xmmol amino groups) is dissolved in 25 ml of 1M HEPES buffer at pH 7.4 (Pierce, Rockford, Ill.) to obtain a 20PC solution. In a separate container, 0.35×mmol of MPEGCM (Mw=10 kDa; MethoxyPEG with carboxyl groups at the terminal, from Lysan bio; Arab, Ala.) is dissolved in 60 ml of 80% ethanol with 20 mM MES pH=4.7 (1200 ul of 1M MES added to 60 ml), 0.5×mmol of NHS added (Mw=115.14), and once dissolved Xmmol EDC (mw=191.71; 10.43 mmol) is added while stirring and allowing the activation to proceed for 20 minutes. Activated MPEGCM is added directly to the 20PC solution, allowing for the reaction to continue for 2 hours and amino groups measured by the TNBS assay to insure that 35% saturation of amino groups. If not, the appropriate amount of activated MPEGCM is added. This is the 20PCPEG1035DA solution. Size exclusion chromatography using a TosohG4000WXL column (0.79×30 cm) with phosphate buffered saline (PBS; 11.9 mM phosphate, 137 mM NaCl, 2.7 mMKPO₄, pH 7.4) containing 15% Acetonitrile elution at a flow rate of 0.6 ml/min is performed. The retention time of is expected to be consistent with a hydrodynamic diameter of approximately 14-17 nm. 5×mmol EDTA dianhydride is added (Mw=256; Sigma Chem Co., St Louis, Mo. Cat#332046) followed by 200 uL TEA. The reaction is titrated slowly with 10 N NaOH to pH 7.1 and stirred for 4 hours. Using the TNBS reaction, it will be confirmed no amino groups remain indicative of a complete reaction, and that the 20PCPEG1035DAEDTA product is made. Size exclusion chromatography is performed using a TosohG4000WXL column (0.79×30 cm) with phosphate buffered saline (PBS; 11.9 mM phosphate, 137 mM NaCl, 2.7 mMKPO₄, pH 7.4) containing 15% Acetonitrile elution at a flow rate of 0.6 ml/min. The retention time is expected to be consistent with a 17-21 nm molecular diameter. The resulting 20PCPEG1035DAEDTA is washed with 15 volumes of water using a 100,000 MWCO ultrafiltration cartridge (UFP-100-E-5A; GE-Amersham) and lyophilized.

### Examples of Compositions with a Co-Polymer as the Polymeric Backbone

### Example 19

Synthesis of 20PLPEG1055DAPEI4NTAZn (Lot#20080421a): a) 5 mL or 2 g equivalent of 20PL (Q4926 SAFC lot# 018K7775; DP=126; 2 g was found to contain 4.60 mmol amino groups by TNBS analysis) was dissolved in 25 ml of 1 M HEPES. This is the 20PL solution. b) In a separate container, 20 g of MPEGCM (Mw=10 kDa; 2.0 mmol; SunBright; ME-100HS; lot#M62503; clean in solution) was dissolved in 60 ml of 80% ethanol with 20 mM MES pH=4.7 (1.2 ml of 1M MES added to 60 ml), 500 mg of NHS (mw=115.14; 4.35 mmol) was added, once dissolved 2.0 g EDC (mw=191.71; 10.43 mmol) was added while stirring. Activation was allowed to proceed for 20 minutes and the activated MPEGCM was added directly to 20PL solution in a. The pH was adjusted to slowly to 7.1 using 10N NaOH one drop at a time, and allowed to react for 2 hours. After 2 hours, amino group analysis by TNBS indicated a total of 1.92 mmol remains indicating 58% MPEG saturation. This is the 20PLPEG1055DA solution. c) Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min (or approximately 22.2 nm molecular diameter) and also showed about 95% incorporation of PEG. d) Succinic Anhydride (2 g; 20 mmol) was added to the 20PLPEG1055DA solution, followed by 200 uL TEA. The reaction was slowly titrated with 10 N NaOH to pH 7.1 and stirred for 4 hours. After 4 hours, no remaining amino groups were detectable by TNBS analysis. Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min using a refractive index detector (or approximately 22.2 nm molecular diameter). The 20PLPEG1040DASA product was washed with 15 volumes of water using a 100 kDa MWCO ultrafiltration cartridge (UFP-100-E-5A; GE-Amersham) and lyophilized giving 13.1 g. e) 25 ml PEI4 (Branched Polyethyleneimine; Mw=400Da; Sigma Chem Co. St Luis Mo.) was dissolved in 25 ml of 1M HEPES and the pH was adjusted to pH 7.4 using about 40 mL of 6N HCl. f) 20PLPEG1055DASA (6.9 g; 1.0 mmol carboxyl) was dissolved in 30 ml of 20 mM MES, 230 mg NHS (mw=115.09; 2 mmol) was added, followed by 1.0 g EDC (mw=191.71; 5.2 mmol). The pH went up slowly but was maintained to 4.7 by adding HCl. After 20 minutes, this solution was added to solution in step e. After 2 hours, the reaction mixture was washed with volumes of water using a 100 kDa MWCO ultrafiltration cartridge (UFP-100-E-5A; GE-Amersham and lyophilized) and lyophilized giving 6.0 g of 20PLPEG1055DAPEI. Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.6 min (or approximately 23.1 nm molecular diameter). g) The amino group content of 20PLPEG1055DAPEI4 was measured by TNBS and found to be 0.186 umol NH2/mg. 20PLPEG1055DAPEI4 (2 g with 0.37 mmol amino) was dissolved in 30 ml of 1 M HEPES and Succinic Anhydride (2 g; MW=100.07) was added. The reaction was slowly titrated with 10 N NaOH to pH 7.1 and stirred for 2 hours. After 2 hours, the total amino groups was measured by TNBS and found to be 0 umol. The reaction mixture was washed with 15 volumes of water using a 100 kDa MWCO ultrafiltration cartridge (UFP-100-E-5A; GE-Amersham) and lyophilized giving 1.9 g of 20PLPEG1055DAPEI4SA. h) NTA-amine (2 g; Nalpha,Nalpha,-Bis(carboxymethyl)-L-Lysine; Mw=262.26+50% impurity, up to 2 mol water and 10% inorganic) equivalent to ˜4 mmol was dissolved in 10 ml of 1M HEPES. Seventeen ml of 0.5M ZnCl was added to the NTA-amine and adjusted to pH7.1 with 10N NaOH. The solution was centrifuged, supernatant was collected, and total amino groups were determined by TNBS. Actual amino group measurement by TNBS indicated that there was a total of 3.07 mmol in the supernatant. i) 20PLPEG1055DAPEI4SA (1.9 g; theoretical primary amine is 0.35 mmol with another 0.18 secondary amine not detected by TNBS which were all converted to carboxyl in step g) was activated by dissolving it in 20 ml of 20 mM MES, adding 150 mg NHS (mw=115.09; 1.3 mmol), followed by adding 600 mg EDC (mw=191.71; 3.1 mmol, pH is maintained to below 5.4. After 20 minutes, activated 20PLPEG1055DAPEI4SA was added to 10 ml NTA supernatant and the pH of the solution was adjusted to 7.1 with 10N NaOH. After 2 hours, the pH was adjusted to pH5 with 6N HCl and 600 mg EDC (mw=191.71; 3.1 mmol) was further added. After 20 minutes, the pH was adjusted back to 7.1. After 1 hour, total amino group amino groups were measured by TNBS and found to be 1.74 mmol indicating that 1.33 mmol of NTA-amine was incorporated into 1.9 g carrier. The reaction mixture was washed with 15 volumes of water using a 100 kDa MWCO ultrafiltration cartridge (UFP-100-E-5A; GE-Amersham), filter-sterilized (0.2 um polysulfone filter; Nalgene; Rochester, N.Y.) and lyophilized giving 1.96 g of 20PLPEG1055DAPEI4NTAZn (lot#20080421a). j) Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approximately 20.0 nm molecular diameter. One mg/ml was analyzed by TNBS and contained 0 nmol/mg. This carrier (Lot#20080421a) did not pick up any additional zinc, thus, zinc saturation was maintained during synthesis.

### Example 20

Synthesis of 20PLPEG1055DAPEI8NTA (Lot#20080421b): a) 5 mL or 2 g equivalent of 20PL (Q4926 SAFC lot# 018K7775; DP=126; 2 g was found to contain 4.60 mmol NH2 as determined by TNBS analysis) was dissolved in 25 ml of 1 M HEPES. This is the 20PL solution. b) In a separate container, 20 g of MPEGCM (Mw=10 kDa; 2.0 mmol; SunBright; ME-100HS; lot#M62503; clean in solution) was dissolved in 60 ml of 80% ethanol with 20 mM MES pH=4.7 (1.2 ml of 1M MES added to 60 ml), 500 mg of NHS (mw=115.14; 4.35 mmol) was added, and once dissolved, 2.0 g EDC (mw=191.71; 10.43 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. After 2 hours, amino group analysis by TNBS indicated that 1.92 mmol total amino group remains indicating 58% MPEG saturation. This is the 20PLPEG1055DA solution. c) Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately 22.2 nm molecular diameter and also showing 95% incorporation of MPEG. d) Succinic Anhydride (2 g; 20 mmol) was added to the 20PLPEG1055DA solution, followed by 200 uL TEA. The reaction was slowly titrated with 10 N NaOH to pH 7.1 and stirred for 4 hours. After 4 hours, amino groups were measured and found to be 0 umol. Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min using a refractive index detector (or approximately 22.2 nm molecular diameter). The resulting 20PLPEG1040DASA was washed with 15 volumes of water using a 100 kDa MWCO ultrafiltration cartridge (UFP-100-E-5A; GE-Amersham) and lyophilized giving 13.1 g. e) 25 ml PEI8 (Branched Polyethyleneimine; Mw=800Da; Sigma Chem Co. St Luis Mo.) was dissolved in 25 ml of 1M HEPES and pH was adjusted to 7.4 using about 40 mL of 6N HCl. f) 20PLPEG1055DASA (3.1 g) was dissolved in 15 ml of 20 mM MES, 115 mg NHS (mw=115.09; 1 mmol) was added, followed by 500 mg EDC (mw=191.71; 2.6 mmol). The pH went up slowly but was maintained to 4.7 by adding HCl. After 20 minutes, this solution was added to solution in step e. After 2 hours, the reaction mixture was washed with 10 volumes of water using a 100,000 MWCO ultrafiltration cartridge (UFP-100-E-5A; GE-Amersham) and lyophilized giving 2.58 g of 20PLPEG1055DAPEI8 Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.4 min or approximately 24.8 nm molecular diameter. g) The amino group content of 20PLPEG1055DAPEI8 was analyzed using TNBS and found to be 0.296 umol NH2/mg. 20PLPEG1055DAPEI8 (2 g with 0.592 mmol amino) was dissolved in 30 ml of 1 M HEPES and Succinic Anhydride (3 g; MW=100.07; 30 mmol) was added. The reaction was slowly titrated with 10 N NaOH to pH 7.1 and stirred for 2 hours. After 2 hours, the amino groups were measured by TNBS and found to be 0 umol. The reaction mixture was washed with 15 volumes of water using a 100 kDa MWCO ultrafiltration cartridge (UFP-100-E-5A; GE-Amersham) and lyophilized giving 1.8 g of 20PLPEG1055DAPEI8SA. h) Two grams of NTA-amine (Nalpha,Nalpha,-Bis(carboxymethyl)-L-Lysine; Mw=262.26+50% impurity, up to 2 mol water and 10% inorganic) or approximately 4 mmol was dissolved in 10 ml of 1M HEPES, 17 ml of 0.5M ZnCl was added, and pH was adjusted to 7.1 using 10N NaOH. The solution was centrifuged, the resulting supernatant was collected, and total amino group measurement by TNBS. The supernatant contained a total of 6.14 mmol amino groups. i) 20PLPEG1055DAPEI8SA (1.8 g; theoretical primary amine was 0.53 mmol with another 0.26 secondary amine not detected by TNBS which were all converted to carboxyl in step g) was dissolved in 20 ml of 20 mM MES, 300 mg NHS (mw=115.09; 2.6 mmol) was added, followed by 1.2 mg EDC (mw=191.71; 6.2 mmol). During the 20 minute reaction, pH was maintained to about 5.4 using HCl. After 20 minutes, the activated 20PLPEG1055DAPEI8SA solution was added to 10 ml NTA-amine supernatant in step h and the pH of the solution was adjusted to 7.1 with 10N NaOH. After 2 hours, the pH was adjusted to ˜pH5 with 6N HCl and 1.2 g EDC (mw=191.71; 6.2 mmol) was further added. After 20 minutes reaction, the pH was adjusted back to 7.1. After 1 hour, total amino groups were measured by TNBS and found to be 4.08 mmol amino groups, indicating that 2.06 mmol of NTA-amine was incorporated into 1.8 g carrier. The reaction mixture was washed with 10 volumes of water using a 100 kDa MWCO ultrafiltration cartridge (UFP-100-E-5A; GE-Amersham), filter-sterilized (0.2 um polysulfone filter; Nalgene; Rochester, N.Y.) and lyophilized (1.55 g; 20PLPEG1055DAPEI8NTAZn; lot#20080421b). j) Size exclusion chromatography using a TosohG4000WXL column (0.79×30 cm) and elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.4 min or approximately 24.8 nm molecular diameter. TNBS indicated that the product 20PLPEG1055DAPEI8NTAZn has 0 nmol NH2/mg.

### Example 21

Synthesis of 20PLPEG550DAPEI4NTAZn (lot#20080603c): a) 15 mL or 6 g equivalent of 20PL (Q4926 SAFC lot# 018K7775; DP=126; 2 g was found to contain 4.72 mmol NH2 by TNBS analysis) was dissolved in 135 ml of 1 M HEPES. This is the 20PL solution. b) In a separate container, 45 g of MPEGCM (Mw=5 kDa; 9.0 mmol; Laysan Bio; lot#108-41; clear solution) was dissolved in 150 ml of 80% ethanol with 10 mM MES pH=4.7 (1.5 ml of 1M MES added to 150 ml) and 2.25 g of NHS (mw=115.14; 19.6 mmol) was added, once dissolved 4.5 g EDC (mw=191.71; 23.5 mmol) was added while stirring. Activation was allowed to proceed for 20 minutes and the activated MPEGCM was added directly to the 20PL solution in step a. The pH was adjusted to pH 7.1 slowly with 10N NaOH one drop at a time, and allowed to react for 2 hours. After 2 hours, amino group analysis by TNBS indicated that 2.16 mmol remained, indicating 54% MPEG saturation. This is the 20PLPEG550DA solution. c) Size exclusion chromatography using a TosohG4000WXL column (0.79×30 cm), elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min (or approximately 14.4 nm molecular diameter) and also showed 95% incorporation of PEG. d) Succinic Anhydride (6 g; 20 mmol) was added to 20PLPEG550DA solution followed by 600 uL TEA. The reaction was slowly titrated with 10 N NaOH to pH 7.1 and stirred for 4 hours. After 4 hours, total amino groups was measured by TNBS and found to be 0 umol. Size exclusion chromatography using a TosohG4000WXL column (0.79×30 cm), elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.3 min or approximately 17.6 nm molecular diameter. e) The reaction mixture containing 20PLPEG550DASA was concentrated to 400 ml and washed with 15 changes of water in a 100 kDa MWCO ultrafiltration cartridge (UFP-100-E-5A), filter-sterilized (0.2 um polysulfone filter; Nalgene, Rochester, N.Y.) and lyophilized yielding 31 g of 20PLPEG550DASA (Lot#20080523). f) 10 ml PEI4 (Branched Polyethyleneimine; Mw=400Da; Sigma Chem Co. St Luis Mo.) was dissolved in 20 ml of 1M HEPES and the pH was adjusted to 7.4 using approximately 16 mL of 6N HCl. g) 20PLPEG550DASA (7.7 g; 1.2 mmol carboxyl) was dissolved in 30 ml of 20 mM MES, 260 mg NHS (mw=115.09; 2.3 mmol) was added, followed by 1.2 g EDC (mw=191.71; 6.3 mmol). After 20 minutes, the activated 20PLPEG550DASA was added to the PEI4 solution. After 2 hours, the pH of the reaction mixture was adjusted to 5.0 using 6N HCl and followed by addition of 1.2 g EDC (mw=191.71; 6.3 mmol). After 20 min activation, the pH was adjusted back to pH7.2 with 10N NaOH. h) The reaction mixture containing 20PLPEG550DAPEI4 was concentrated to 100 ml, washed with 10 changes of water using a 100 kDa MWCO ultrafiltration cartridge (UFP-100-E-5A; GE-Amersham), filter-sterilized (0.2 um polysulfone filter, Nalgene, Rochester, N.Y.)) and lyophilized giving 7.2 g (Lot#20080603). i) Size exclusion chromatography using a TosohG4000WXL column (0.79×30 cm), elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approximately 20.6 nm diameter. j) Sample 20PLPEG550DAPEI4 was analyzed by TNBS and found to contain 204 nmol primary amino groups/mg. k) 20PLPEG550DAPEI4 (2.0 g; 0.3 mmol amino) was dissolved in 30 ml of 1 M HEPES and Succinic Anhydride (2 g; MW=100.07) was added. The reaction was slowly titrated with 10 N NaOH to pH 7.1 and stirred for 2 hours. After 2 hours, amino groups were found to be 0 umol. The reaction mixture containing 20PLPEG550DAPEI4SA was washed with 15 changes of water in 100 kDa MWCO ultrafiltration cartridge (UFP-100-E-5A). l) Two grams of NTA-amine (Nalpha,Nalpha,-Bis(carboxymethyl)-L-Lysine; Mw=262.26+50% impurity, up to 2 mol water and 10% inorganic) or approximately 4 mmol was dissolved in 5 ml of 1M HEPES. Nine ml of 0.5M ZnCl was added to the NTA and adjusted to pH7.1 with 10N NaOH, followed by centrifugation. The supernatant was collected and the amino groups were determined by TNBS analysis. The total amino groups in the supernatant were found to be 4.86 mmol. m) 20PLPEG550DAPEI4SA (2 g; 0.5 mmol carboxyl) was activated by dissolving in 20 ml of 20 mM MES, adding 160 mg NHS (mw=115.09; 1.3 mmol), followed by 650 mg EDC (mw=191.71; 3.4 mmol). During the 20 minute activation reaction, pH was maintained to 5.4. After 20 minutes, the activated 20PLPEG550DAPEI4SA was added to 10 ml NTA-amine supernatant and adjusted to pH 7.1 with 10N NaOH. After 2 hours, the pH was adjusted to 5 with 6N HCl and 650 mg EDC (mw=191.71; 3.4 mmol) was added. After the 20 minute reaction, the pH was adjusted back to pH7.1. After 1 hour, amino group analysis indicated that 4.86 mmol amino group remained, with 0.69 mmol incorporated to 2.0 g carrier. n) Size exclusion chromatography using a TosohG4000WXL column (0.79×30 cm), elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.8 min or approximately 21.4 nm molecular diameter. o) Sample 20PLPEG550DAPEI4NTAZn was washed with 15 volumes of water (0.2 um polysulfone filter, Nalgene, Rochester, N.Y.), filter sterilized (0.2 um polysulfone filter; Nalgene; Rochester, N.Y.), and lyophilized yielding 1.26 g (Lot#20080603c) Amino group analysis by TNBS indicated that 20PLPEG550DAPEI4NTAZn contained 0.0 nmol amino group/mg.

### Example 22

Synthesis of 20PLPEG550DAPEI8NTAZn (lot#20080604c): a) 15 ml or 6 g equivalent of 20PL (Q4926 SAFC lot# 018K7775; DP=126; 2 g was found to contain 4.72 mmol NH2 by TNBS assay) was dissolved in 135 ml of 1 M HEPES. This is the 20PL solution. b) In a separate container, 45 g of MPEGCM (Mw=5 kDa; 9.0 mmol; Laysan Bio; lot#108-41; clear solution) was dissolved in 150 ml of 80% ethanol with 10 mM MES pH=4.7 (1.5 ml of 1M MES added to 150 ml), 2.25 g of NHS (mw=115.14; 19.6 mmol) was added, once dissolved, 4.5 g EDC (mw=191.71; 23.5 mmol) was added while stirring. Activation was allowed to proceed for 20 minutes. The activated MPEGCM was added directly to 20PL solution in step a. The pH was adjusted to pH 7.1 slowly with 10N NaOH one drop at a time, and allowed to react for 2 hours. After 2 hours, amino group analysis by TNBS indicated that 2.16 mmol amino groups remained indicating 54% MPEG saturation. This is the 20PLPEG550DA solution. c) Size exclusion chromatography using a TosohG4000WXL column (0.79×30 cm), elution with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min or approximately 14.4 nm molecular diameter and also showed 95% incorporation of MPEG. d) Succinic Anhydride (6 g; 20 mmol) was added to the 20PLPEG550DA solution followed by 600 uL TEA. The reaction was slowly titrated with 10 N NaOH to pH 7.1 and stirred for 4 hours. The amino groups were measured by TNBS and found to be 0 umol. Size exclusion chromatography as above showed retention time of 12.3 min or approximately 17.6 nm diameter after succinylation. e) The reaction mixture containing 20PLPEG550DASA was concentrated to 400 ml and washed with 15 changes of water in a 100,000 MWCO ultrafiltration cartridge (UFP-100-E-5A), filter-sterilized (0.2 um polysulfone filter, Nalgene, Rochester, N.Y.) and lyophilized yielding giving 31 g of 20PLPEG550DASA (Lot#20080523). g) 20 ml PEI8 (Branched Polyethyleneimine; Mw=800Da; Sigma Chem Co. St Luis Mo.) was dissolved in 20 ml of 1M HEPES and the pH was adjusted to 7.4 using approximately 32 mL of 6N HCl. h) 20PLPEG550DASA (7.7 g; 1.2 mmol carboxyl) was dissolved in 30 ml of 20 mM MES, 260 mg NHS (mw=115.09; 2.3 mmol) was added, followed by 1.2 g EDC (mw=191.71; 6.3 mmol). The pH went up slowly but was maintained below 5.5 using HCl during the 20 minute activation reaction. After 20 minutes, the activated 20PLPEG550DASA was added to solution in step g. After 2 hours, the pH was adjusted to down to 5.0 with 6N HCl, followed by addition of 1.2 g EDC (mw=191.71; 6.3 mmol). After 20 min activation, the pH was adjusted back to pH7.2 with 10N NaOH. i) The reaction mixture containing 20PLPEG550DAPEI8 was concentrated to 100 ml and washed with 15 changes of water in 100 kDa MWCO ultrafiltration cartridge (UFP-100-E-5A), filter-sterilized (0.2 um polysulfone filter, Nalgene, Rochester, N.Y.) and lyophilized yielding 7.6 g of 20PLPEG550DAPEI8 (Lot#20080604). j) Analysis by Size Exclusion chromatography using TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately a 22.2 nm molecular diameter. The product 20PLPEG550DAPEI8 was analyzed by TNBS and found to contain 304 nmol amino groups/mg. k) 20PLPEG550DAPEI8 (2 g; 0.3 mmol carboxyl) was dissolved in 30 ml of 1 M HEPES, succinic anhydride (2 g; MW=100.07) was added, the reaction was slowly titrated with 10 N NaOH to pH 7.1, and stirred for 2 hours. After 2 hours, the total amino groups were measured and found to be 0.0 umol. The reaction mixture containing 20PLPEG550DAPEI8SA was washed with 15 changes of water in a 100 kDa MWCO ultrafiltration cartridge (UFP-100-E-5A) and lyophilized. l) Three grams of NTA-amine (Nalpha,Nalpha, -Bis(carboxymethyl)-L-Lysine; Mw=262.26+50% impurity, up to 2 mol water and 10% inorganic) of approximately 4 mmol was dissolved in 8 ml of 1M HEPES and 14 ml of 0.5M ZnCl was added to the NTA-amine and adjusted to pH7.1 with 10N NaOH. The solution was centrifuged, supernatant was collected, and the total amino groups were determined TNBS. Amino groups were found to be 3.07 mmol. m) 20PLPEG550DAPEI8SA (2 g; 0.5 mmol carboxyl) was activated by dissolving in 20 ml of 20 mM MES, adding 160 mg NHS (mw=115.09; 1.3 mmol), followed by 650 mg EDC (mw=191.71; 3.4 mmol). During the 20 minute activation reaction, pH was maintained below 5.5. After 20 minutes, the activated 20PLPEG550DAPEI8SA was added to 10 ml NTA supernatant from step 1 and the pH was adjusted to 7.1 with 10N NaOH. After 2 hours, the pH was adjusted to pH5 with 6N HCl and 650 mg EDC (mw=191.71; 3.4 mmol) was added. After a 20 minute reactivation reaction, the pH was adjusted back to 7.1. After 1 hour, total amino groups were measured by TNBS and found to be 7.85 mmol, indicating that 0.62 mmol NTA was incorporated to 2.0 g carrier. n) Analysis by Size Exclusion chromatography using a TosohG4000WXL column (0.79×30 cm), eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately a 22.2 nm molecular diameter. o) Sample 20PLPEG550DAPEI8NTAZn was washed with 15 changes of water in a 100 kDa MWCO ultrafiltration cartridge (UFP-100-E-5A), filter-sterilized (0.2 um polysulfone filter, Nalgene, Rochester, N.Y.) and lyophilized yielding 1.27 g of 20PLPEG550DAPEI8NTAZn (lot#20080604c). TNBS analysis indicated that 20PLPEG550DAPEI8NTAZn (lot#20080604c) contained 0 nmol/mg primary amino groups.

### Example 23

Synthesis of 20PLPEG550DAPEI12NTAZn (lot#20080605c): a) 15 mL or 6 g equivalent of 20PL (Q4926 SAFC lot# 018K7775; DP=126; 2 g was found to contain 4.72 mmol NH2 as determined by TNBS assay) was dissolved in 135 ml of 1 M HEPES. This is the 20PL solution. b) In a separate container, 45 g of MPEGCM (Mw=5 kDa; 9.0 mmol; Laysan Bio; lot#108-41; clear in solution) was dissolved in 150 ml of 80% ethanol with 10 mM MES pH=4.7 (1.5 ml of 1M MES added to 150 ml), 2.25 g of NHS (mw=115.14; 19.6 mmol) was added, once dissolved 4.5 g EDC (mw=191.71; 23.5 mmol) was added while stirring. Activation was allowed to proceed for 20 minutes and the activated MPEGCM was added directly to 20PL solution in step a. The pH was adjusted to pH 7.1 slowly with 10N NaOH one drop at a time, and allowed to react for 2 hours. Amino group analysis by TNBS indicated a total amino group of 2.16 mmol, indicating 54% MPEG saturation. This is the 20PLPEG550DA solution. c) Size exclusion chromatography of 20PLPEG550DA using a TosohG4000WXL column (0.79×30 cm) and eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min or approximately a 14.4 nm molecular diameter and also showed 95% incorporation of PEG. d) Succinic Anhydride (6 g; 20 mmol) was added to 20PLPEG550DA and followed by 600 uL TEA. The reaction was slowly titrated with 10 N NaOH to pH 7.1 and stirred for 4 hours. After 4 hours, the total amino groups were measured by TNBS and found to be 0 umol. The product 20PLPEG550DASA was analyzed by size exclusion chromatography as above and found to have a retention time of 12.3 min or approximately a 17.6 nm in diameter. e) The reaction mixture containing 20PLPEG550DASA was concentrated to 400 ml and washed with 15 changes of water in a 100 kDa MWCO ultrafiltration cartridge (UFP-100-E-5A), filter-sterilized (0.2 um polysulfone filter, Nalgene, Rochester, N.Y.), and lyophilized yielding 31 g of 20PLPEG550DASA (lot#20080523). g) 50 ml PEI12 (Branched Polyethyleneimine; Mw=1200Da; Sigma Chem Co. St Luis Mo.) was dissolved in 20 ml of 1M HEPES, pH was adjusted to 7.4 using approximately 45 mL of 6N HCl. h) 20PLPEG550DASA (7.7 g; 1.2 mmol carboxyl) was dissolved in 30 ml of 20 mM MES, 260 mg NHS (mw=115.09; 2.3 mmol) was added, followed by 1.2 g EDC (mw=191.71; 6.3 mmol). The pH went up slowly but maintained to below 5.5 using HCl. After 20 minutes, the activated 20PLPEG550DASA was added to solution in step g. After 2 hours, the pH was adjusted to pH 5.0 with 6N HCl and followed by addition of 1.2 g EDC (mw=191.71; 6.3 mmol). After 20 min activation, the pH was adjusted back 7.2 with 10N NaOH. i) After 2 hours, the reaction mixture containing 20PLPEG550DAPEI12 was concentrated to 100 ml and washed with 15 changes of water in a 100 kDa MWCO ultrafiltration cartridge (UFP-100-E-5A), filter-sterilized (0.2 um polysulfone filter, Nalgene, Rochester, N.Y.) and lyophilized yielding 7.8 g of 20PLPEG550DAPEI12 (lot#20080605). j) Analysis of 20PLPEG550DAPEI12 by size exclusion chromatography using a TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.7 min or approximately 22.2 nm molecular diameter. TNBS analysis indicated that 20PLPEG550DAPEI12 contained 448 nmol amino group/mg. k) 20PLPEG550DAPEI12 (2 g; 0.3 mmol carboxyl) was dissolved in 30 ml of 1M HEPES and succinic anhydride (2 g; MW=100.07) was added. The reaction mixture was slowly titrated with 10 N NaOH to pH 7.1 and stirred for 2 hours. After 2 hours the total amino groups were measured by TNBS and found to be 0 umol. The reaction mixture containing 20PLPEG550DAPEI12SA was washed with 15 changes of water in 100,000 MWCO ultrafiltration cartridge (UFP-100-E-5A), filter-sterilized (0.2 um polysulfone filter, Nalgene, Rochester, N.Y.) and lyophilized. l) Four grams of NTA-amine (Nalpha,Nalpha, -Bis(carboxymethyl)-L-Lysine; Mw=262.26+50% impurity, up to 2 mol water and 10% inorganic) of approximately 4 mmol of was dissolved in 10 ml of 1M HEPES, 18 ml of 0.5M ZnCl was added, and the pH was adjusted to 7.1 with 10N NaOH. The NTA amine solution was centrifuged, supernatant was collected, and the total amino group in the supernatant was determined by TNBS and indicated that there was 3.07 mmol total amino groups. m) 20PLPEG550DAPEI12SA (2 g; 0.5 mmol carboxyl for primary amine succinate) was dissolved in 20 ml of 20 mM MES, 160 mg NHS (mw=115.09; 1.3 mmol) was added, followed by 650 mg EDC (mw=191.71; 3.4 mmol). During the 20 minute activation reaction, pH was maintained below 5.5. After 20 minutes, activated 20PLPEG550DAPEI12SA solution was added to 10 ml NTA supernatant and the pH was adjusted to pH 7.1 using 10N NaOH. After 2 hours, the pH was adjusted back to 5.5 using 6N HCl, and 650 mg EDC (mw=191.71; 3.4 mmol) was added. After 20 minute reactivation reaction, the pH was adjusted back to pH7.1 with 10N NaOH. After 1 hour, amino group analysis by TNBS indicated that 2.15 mmol amino groups remained, indicating that 0.92 mmol was incorporated to 2.0 g carrier. Sample 20PLPEG550DAPEI12NTAZn was washed with 15 changes of water in a 100,000 MWCO ultrafiltration cartridge (UFP-100-E-5A), filter-sterilized (0.2 um polysulfone filter, Nalgene, Rochester, N.Y.) and lyophilized yielding 2.5 g (lot#20080605c). n) Analysis of 20PLPEG550DAPEI12NTAZn by size exclusion chromatography using a TosohG4000WXL column (0.79×30 cm), eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 11.9 min or approximately 20.6 nm molecular diameter. TNBS analysis indicated that 20PLPEG550DAPEI12NTAZn contained 0 nmol amino group/mg.

### Examples of Compositions with a Polymeric Backbone Derived from a Non-Biological Monomer

### Example 24

Synthesis of 18PEIPEG1030DANTAZn (Lot#20080804b): a) 3.2 g of 18PEI (408700 Aldrich lot# 07326LH; DP=126) was titrated to pH7.4 with 2.9 ml of 6N HCl and made up to 32 ml of 1 M HEPES. Primary amino group by TNBS analysis was found to be 10.24 mmol NH2/3.22 g. This is the 18PEI solution. b) In a separate container, 15 g of MPEGCM (Mw=10 kDa; 1.45 mmol; Laysan; lot#108-108; clear in solution) was dissolved in 45 ml of 80% ethanol with 10 mM MES pH=4.7 (600 ul of 1M MES added to 60 ml), 375 mg of NHS (mw=115.14; 3.26 mmol) was added, once dissolved 1.5 g EDC (mw=191.71; 7.82 mmol) was added while stirring. Activation was allowed to proceed for 20 minutes and the activated MPEGCM was added directly to 18PEI solution in step a. The pH was adjusted to pH 7.1 slowly with 10N NaOH one drop at a time, and allowed to react for 2 hours. After 2 hours, the pH was adjusted back to 5.5 with 6N HCl and 1.5 g EDC (mw=191.71; 7.82 mmol) was added. After a 20 minute reaction, the pH was adjusted back to pH 7.1. After 2 hours, amino group analysis by TNBS indicated 7.53 mmol amino group remained, indicating 26% PEG saturation. This is the 18PEIPEG1030DA solution. c) The reaction mixture containing 18PEIPEG1030DA was washed with 10 volumes of 80% ethanol using a 3 kDa MWCO filter cartridge (UFP-10-E-5A; GE-Amersham), filter-sterilized (0.2 um polysulfone filter, Nalgene, Rochester, N.Y.), and lyophilized (7.76 g; 18PEIPEG1030DA; Lot#20080804). Analysis of 18PEIPEG1030DA by size exclusion chromatography using a TosohG3000WXL column (0.79×30 cm), eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 9.75 min by UV220 nm or approximately 9.5 nm in diameter. 18PEIPEG1030DA was analyzed by TNBS and found to contain 310 nmol NH2/mg. d) 18PEIPEG1030DA (1.5 g; 0.47 mmol amino) was dissolved in 30 ml of 1M HEPES, succinic anhydride (2 g; 10 mmol) was added, the solution was slowly titrated with to pH 7.1 using 10 N NaOH, and stirred for 4 hours. After 4 hours, amino groups was measured by TNBS and found to be 0 umol. The reaction mixture containing 18PEIPEG1030DASA was washed with 10 volumes of 80% ethanol using a 100 kDa MWCO filter cartridge (UFP-100-E-5A; GE-Amersham), and concentrated in 80% ethanol and collected. Analysis by size exclusion chromatography using a TosohG4000WXL column (0.79×30 cm), eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.8 min by UV220 nm or approximately 14.3 nm in diameter. e) Half gram of NTA-amine (Nalpha,Nalpha,-Bis(carboxymethyl)-L-Lysine; Mw=262.26+unknown % impurity, up to 2 mol water and 10% inorganic) of about 1.9 mmol was dissolved in 3 ml of 1M HEPES, 3 ml of 0.5M ZnCl was added and the pH was adjusted to pH7.1 with 10N NaOH. The solution was centrifuged, supernatant was collected, and the total amino groups of the supernatant was measured by TNBS and found to be 1.71 mmol. f) 18PEIPEG1030DASA (200 ml from D; 0.5 mmol carboxyl from primary amino by stoichiometry not including secondary or tertiary amine) was buffered with 2 ml of 1M MES, pH 4.7 and activated by adding 230 mg NHS (mw=115.09; 2.0 mmol) followed by 1.15 g EDC (mw=191.71; 6.0 mmol). The pH was maintained below 5.5 using HCl. After 20 minutes the activated 18PEIPEG1030DASA was added to 13 ml of NTA-Zn supernatant in step e and the pH was adjusted to 7.1 using 10N NaOH. The solution was magnetically stirred overnight. The next day, total amino groups were measured by TNBS and found to be 0.47 mmol, indicating that the total amino groups incorporated into the carrier was 1.24 mmol. g) The reaction mixture containing 18PEIPEG1030DANTAZn was washed with 10 volumes of 80% ethanol using a 100 kDa MWCO filter cartridge (UFP-100-E-5A; GE-Amersham) followed by 10 volume of water, filter-sterilized (0.2 um polysulfone filter, Nalgene, Rochester, N.Y.) and lyophilized yielding 1.65 g (18PEIPEG1030DANTAZn; lot#20080804b). Analysis by Size Exclusion chromatography using a TosohG4000WXL column (0.79×30 cm) eluted with phosphate buffered saline (PBS; 11.9 mM phosphate, 137 mM NaCl, 2.7 mMKPO₄, pH 7.4) containing 15% Acetonitrile at a flow rate of 0.6 ml/min showed a retention time of 12.2 min or approximately 19.1 nm in diameter. TNBS analysis indicated that the 18PEIPEG1030DANTAZn had only 2 nmol primary amino group/mg.

### Example 25

Synthesis of 15PALPEG1035DAEDTA: This composition has polyallylamine hydrochloride (Sigma Chem. Co. St Luis Mo. Cat#283215, Mw=15 kDa) as the polymeric backbone with 35% of the amino groups occupied with 10 kDa MPEGCM (Mw=10 kDa; SunBright; ME-100HS; lot#M62503; clean in solution) and the remaining amino group occupied with ethyenediaminetetraacetic acid (EDTA). To synthesize 15PALPEG1035DAEDTA, 1.0 g of polyallylamine hydrochloride (with Xmmol amino groups as measured by TNBS) is dissolved in 25 ml of 1M HEPES buffer at pH 7.4 (Pierce, Rockford, Ill.) to obtain a 15PAL solution. In a separate container, 0.35×mmol of MPEGCM is dissolved (Mw=10 kDa; MethoxyPEG with carboxyl group at the terminal, from Lysan bio; Arab, Ala.) in 60 ml of 80% ethanol with 20 mM MES pH=4.7 (1200 ul of 1M MES added to 60 ml), 0.5×mmol of NHS added (Mw=115.14), once dissolved Xmmol EDC (mw=191.71; 10.43 mmol) is added while stirring allowing the activation to proceed for 20 minutes. The activated MPEGCM is added directly to the 15PAL solution, allowing for the reaction to occur for 2 hours and the amino groups measured by TNBS to ensure if 35% saturation of amino groups. Otherwise, more of the appropriate amount of activated MPEGCM is added. This is the 15PALPEG1035DA solution. Size exclusion chromatography is performed using a TosohG4000WXL column (0.79×30 cm) with phosphate buffered saline (PBS; 11.9 mM phosphate, 137 mM NaCl, 2.7 mMKPO₄, pH 7.4) containing 15% Acetonitrile elution at a flow rate of 0.6 ml/min. The retention time is expected to be consistent with approximately a 14-17 nm molecular diameter. 5×mmol EDTA dianhydride (Mw=256; Sigma Chem Co., St Louis, Mo. Cat#332046) is added followed by 200 uL TEA. The reaction is titrated slowly with 10 N NaOH to pH 7.1 and stirred for 4 hours. Using TNBS reaction, it can be confirmed that no amino groups remain indicative of a complete reaction, and that the 15PALPEG1035DAEDTA product is made. Size exclusion chromatography is performed using a TosohG4000WXL column (0.79×30 cm) with phosphate buffered saline (PBS; 11.9 mM phosphate, 137 mM NaCl, 2.7 mMKPO₄, pH 7.4) containing 15% Acetonitrile elution at a flow rate of 0.6 ml/min. The retention time is expected to be consistent with a 17-21 nm molecular diameter. The resulting 15PALPEG1035DAEDTA is washed with 15 volumes of water using a 100,000 MWCO ultrafiltration cartridge (UFP-100-E-5A; GE-Amersham) and lyophilized.

### Example 26

Synthesis of 20PMAPEG1035DANTA: This composition has polymethylmethacrylate (Sigma Chem. Co. St Luis Mo. Cat#81498, Mw=20 kDa) as the polymeric backbone with 35% of the methoxy groups replaced with 10 kDa MPEGAM (Mw=10 kDa MPEG with amino group at the terminal; Sunbio, Orinda, Calif.; Cat#P1AM-10) and the remaining methoxy groups occupied with nitrilotriacetic acid. To prepare this composition all methoxy groups in polymethylmethacrylate (Sigma Chem. Co. St Luis Mo. Cat#81498, Mw=20 kDa) can be removed using Methanolic/KOH to obtain polymethylacrylic acid. Briefly, 2 g of polymethylmethcrylate is dissolved in 25 ml of 10% methanolic KOH for and reflux for 96 h. Once neutralized with HCl and methanol removed by rotary evaporation at 40° C., the solid is dissolved in water and washed with 15 changes of water in a 3 kDa-MWCO ultrafiltration cartridge (UFP-3-E-5A). The resulting product (PMA) is filter-sterilized (0.2 um polysulfone filter, Nalgene, Rochester, N.Y.) and lyophilized to obtain PMA. The starting carboxyl group (Xmmol) is measured in 1.0 g PMA according to the protocol by Kobayahi and Chiba (Analytical biochemistry 1994, vol 219, p 189-194). To synthesize 20PMAPEG1035DANTA, 1.0 g of PMA (with Xmmol carboxyl group) is dissolved in 25 ml of 20 mM MES (2-(N-morpholino)ethanesulfonic acid, Pierce, Rockford, Ill.) buffer pH 4.7 (PS solution). 0.35×mmol of MPEGAM is dissolved in (Mw=10 kDa MPEG with amino groups at the terminal; Sunbio, Orinda, Calif.; Cat#P1AM-10) in 20 ml of 80% ethanol and added to the PMA solution to make a PMAPEG solution. To the PMAPEG solution, Xmmol of NHS (Mw=115.14) and Xmmol EDC (Mw=191.71) is added to the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If pH is below 7.1 the pH is adjusted with 10N NaOH one drop at a time to reach 7.1. The reaction is allowed to proceed for 2 hours and the remaining amino groups of the MPEGAM are measured by TNBS and are expected to be 0, indicating that all 0.35×mmol amino group is used up and conjugated to the PMA. If there are remaining amino group, pH is adjusted to 5 with 6N HCl and Xmmol EDC added (Mw=191.71), and after 20 minutes pH adjusted back to 7.1 and allowed to react overnight. An aliquot of the resulting 20PMAPEG1035 solution is used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with diameter of approximately 13-18 nm. The pH is adjusted down to 5 with 6N HCl and 1.5×mmol EDC added (Mw=191.71) and activated for 20 minutes. After 20 minutes this activated 20PMAPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The 20PMAPEG1035DANTA product is filter-sterilized (using 0.2 um polysulfone filter, Nalgene, Rochester, N.Y.) and lyophilized. 10 mg/ml solution of resulting 20PMAPEG1035DANTA is made and the hydrodynamic diameter is determined by size exclusion chromatography using a TosohG4000WXL column (0.79×30 cm) and phosphate buffered saline as a elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with diameter of approximately 16-20 nm.

### Example 27

Synthesis of 15PACPEG1035DANTA: This composition has polyacrylic acid or PAC (Sigma Chem. Co. St Luis Mo. Cat#81123, Mw=16 kDa) as the polymeric backbone with 35% of the carboxyl groups linked 10 kDa MPEGAM (Mw=10 kDa MPEG with amino group at the terminal; Sunbio, Orinda, Calif.; Cat#P1AM-10) and the remaining carboxyl groups occupied with nitrilotriacetic acid. The starting carboxyl group is measured (Xmmol) in 1.0 g PAC according to the protocol by Kobayahi and Chiba (Analytical biochemistry 1994, vol 219, p 189-194). To synthesize 15PACPEG1035DANTA, 1.0 g of PAC (with Xmmol carboxyl group) is dissolved in 25 ml of 20 mM MES (2-(N-morpholino)ethanesulfonic acid, Pierce, Rockford, Ill.) buffer pH 4.7 (PS solution). 0.35×mmol of MPEGAM is dissolved (Mw=10 kDa MPEG with amino groups at the terminal; Sunbio, Orinda, Calif.; Cat#P1AM-10) in 20 ml of 80% ethanol and added to the PMA solution to make PMAPEG solution. To the PMAPEG solution, Xmmol of NHS (Mw=115.14) and Xmmol EDC (Mw=191.71) are added in solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If the pH is below 7.1 the pH is adjusted with 10N NaOH one drop at a time to reach 7.1. The reaction is allowed to proceed for 2 hours and the remaining amino groups of the MPEGAM are measured by TNBS and is expected to be none indicating that all 0.35×mmol of amino groups are used up and conjugated to the PAC. If there are remaining amino groups in MPEGAM, the pH is adjusted to 5 with 6N HCl and Xmmol EDC (Mw=191.71) is added, after 20 minutes the pH is adjusted back to 7.1 and allowed to react overnight. An aliquot of the resulting 15PACPEG1035 solution is taken and used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with diameter of approximately 12-17 nm. The pH is adjusted down to 5 with 6N HCl and 1.5×mmol EDC added (Mw=191.71) and activated for 20 minutes. After 20 minutes this activated 15PACPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The 15PACPEG1035DANTA product is filter-sterilized (using 0.2 um polysulfone filter, Nalgene, Rochester, N.Y.) and lyophilized. A 10 mg/ml solution of resulting 15PACPEG1035DANTA is made and the hydrodynamic diameter is determined by size exclusion chromatography using TosohG4000WXL column (0.79×30 cm) and phosphate buffered saline as elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with diameter of approximately 15-20 nm.

### Examples of Compositions with a Polymeric Backbone Derived from Polysaccharides

### Example 28

Synthesis of 1000HYPEG1035DANTA: This composition has hyaluronic acid or HY (Sigma Chem. Co. St Luis Mo. Cat#53747, Mw=1000 kDa) as the polymeric backbone with 35% of the carboxyl groups replaced with 10 kDa MPEGAM (Mw=10 kDa MPEG with amino groups at the terminal; Sunbio, Orinda, Calif.; Cat#P1AM-10) and the remaining carboxyl groups occupied with nitrilotriacetic acid. To do this the Hyluronic acid can be used without addition of more carboxyl groups or additional carboxyl groups can be added. To add additional carboxyl groups, the hydroxyl groups in hyluronan can be converted to carboxy groups according to the protocol by Tijsen et al. (Carbohydrate Polymers 2001, vol 45; p 219-226). Briefly, 10 grams of hyluronan is suspended in 100 ml of 90% propanol/water and 4 grams NaOH pellet is added and stirred overnight at 40° C. The next day 10 grams of sodium-monochloro-acetate (Sigma Chem Co. St Luis, Mo. Cat#291773) is added as outlined in Tijsen et al. (Carbohydrate Polymers 2001, vol 45; p 219-226). At the end of the reaction the carboxymethylated hyaluronan is washed with 15 changes of water in a 3 kDa-MWCO ultrafiltration cartridge (UFP-3-E-5A). The resulting product (PMA) is filter-sterilized using (0.2 um polysulfone filter, Nalgene, Rochester, N.Y.) and lyophilized to obtain hyluronan with extra carboxyl groups. Before synthesis of 1000HYPEG1035DANTA, the starting carboxyl group is measured (Xmmol) of 1.0 g HY (this could be HY without additional carboxyl groups or HY modified to have additional carboxyl groups) according to the protocol by Kobayahi and Chiba (Analytical biochemistry 1994, vol 219, p 189-194). To synthesize 1000HYPEG1035DANTA, 1.0 g of HY (with Xmmol carboxyl groups) is dissolved in 25 ml of 20 mM MES (2-(N-morpholino)ethanesulfonic acid, Pierce, Rockford, Ill.) buffer pH 4.7 to obtain HY solution. 0.35×mmol of MPEGAM is dissolved (Mw=10 kDa MPEG with amino group at the terminal; Sunbio, Orinda, Calif.; Cat#P1AM-10) in 20 ml of 80% ethanol and added to the HY solution to make HYPEG solution. To the HYPEG solution, Xmmol of NHS (Mw=115.14) and Xmmol EDC (Mw=191.71) is added of the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If the pH is below 7.1 the pH is adjusted with 10N NaOH one drop at a time to reach 7.1. The reaction is allowed to proceed for 2 hours and the remaining amino groups of the MPEGAM are measured by TNBS and it is expected to be none indicating that all 0.35×mmol amino group is used up and conjugated to the HY. If there are remaining amino group in MPEGAM, the pH is adjusted to 5 with 6N HCl and Xmmol EDC (Mw=191.71) is added, and after 20 minutes the pH is adjusted back the pH to 7.1 and allowed to react overnight to form 1000HYPEG1035. An aliquot of the resulting 1000HYPEG1035 solution is used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with diameter of approximately 30-50 nm. The pH is adjusted down to 5 with 6N HCl and 1.5×mmol EDC is added (Mw=191.71) and activated for 20 minutes. After 20 minutes this activated 1000HYPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The 1000HYPEG1035DANTA product is filter-sterilized (using a 0.2 um polysulfone filter, Nalgene, Rochester, N.Y.) and lyophilized. A 10 mg/ml solution of the resulting 1000HYPEG1035DANTA is made and used to determine the hydrodynamic diameter by size exclusion chromatography using TosohG4000WXL column (0.79×30 cm) and phosphate buffered saline as elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with diameter of approximately 30-70 nm.

### Example 29

Synthesis of 60PGAPEG1035DANTA: This composition has pectin or polygalacturonic acid (Sodiumpolypectate; Sigma Chem. Co. St Luis Mo. Cat#p3889, Mw=60 kDa) as the polymeric backbone with 35% of the carboxyl groups replaced with 10 kDa MPEGAM (Mw=10 kDa MPEGAM has amino group at the terminal; Sunbio, Orinda, Calif.; Cat#P1AM-10) and the remaining carboxyl groups occupied with nitrilotriacetic acid. To do this the pectin can be used without addition of more carboxyl groups or additional carboxyl groups can be added. To add additional carboxyl groups, the hydroxyl groups in pectin (PGA) can be converted to carboxy groups according to the protocol by Tijsen et al. (Carbohydrate Polymers 2001, vol 45; p 219-226). Briefly, 10 grams of pectin is suspended in 100 ml of 90% propanol/water and 4 grams NaOH pellet are added and stirred overnight at 40° C. The next day 10 grams of sodium-monochloro-acetate (Sigma Chem Co. St Luis, Mo. Cat#291773) is added as outlined in Tijsen et al. (Carbohydrate Polymers 2001, vol 45; p 219-226). At the end of the reaction the carboxymethylated pectin is washed with 15 changes of water in a 3 kDa-MWCO ultrafiltration cartridge (UFP-3-E-5A). The resulting product (PMA) is filter-sterilized (0.2 um polysulfone filter, Nalgene, Rochester, N.Y.) and lyophilized to obtain pectin with extra carboxyl groups. Occasionally, pectin will have a significant percent of the carboxyl group blocked with methyl groups. This can be removed using acid to expose all carboxyl groups of pectin. Briefly, 2 g of pectin is dissolved in 100 ml water and the pH is adjusted to 0.5 using concentrated HCl and kept at 80° C. for 2 hours according to Constenla and Lazano (Latin American Applied Research 2003, vol 33, p 91-96). It is neutralized with NaOH and washed with 15 changes of water in a 3 kDa-MWCO ultrafiltration cartridge (UFP-3-E-5A). The resulting product is filter-sterilized using a 0.2 um polysulfone filter (Nalgene, Rochester, N.Y.) and lyophilized. This pectin can be processed to increase the amount of carboxyl groups as described above. 60PGAPEG1035DANTA can be made using any of the pectins (PGA) described above, unprocessed or processed to increase carboxyl groups. Before synthesis of 60PGAPEG1035DANTA, the starting carboxyl group (Xmmol) of 1.0 g PGA is measured (this could be PGA without additional carboxyl groups or PGA modified to have additional carboxyl groups) according to the protocol by Kobayahi and Chiba (Analytical biochemistry 1994, vol 219, p 189-194). To synthesize 60PGAPEG1035DANTA, 1.0 g of PGA (with Xmmol carboxyl group) is dissolved in 50 ml of 20 mM MES (2-(N-morpholino)ethanesulfonic acid, Pierce, Rockford, Ill.) buffer pH 4.7 to obtain PGA solution. 0.35×mmol of MPEGAM is dissolved (Mw=10 kDa MPEG with amino groups at the terminal; Sunbio, Orinda, Calif.; Cat#P1AM-10) in 20 ml of 80% ethanol and added to the PGA solution to make PGAPEG solution. To the PGAPEG solution, Xmmol of NHS (Mw=115.14) and Xmmol EDC (Mw=191.71) are added to the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If the pH is below 7.1 the pH is adjusted with 10N NaOH one drop at a time to reach 7.1. The reaction to is allowed to proceed for 2 hours and the remaining amino group of the MPEGAM by TNBS and is expected to be none indicating that all 0.35×mmol amino group is used up and conjugated to the PGA. If there are remaining amino groups in MPEGAM, the pH is adjusted to 5 with 6N HCl and Xmmol EDC (Mw=191.71) is added, and after 20 minutes the pH is adjusted back to 7.1 and allowed to react overnight to form 60PGAPEG1035. An aliquot of the resulting 60PGAPEG1035 solution is used to determine the hydrodynamic diameter by size exclusion chromatography using a TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with a diameter of approximately 18-30 nm. The pH is adjusted down to 5 with 6N HCl and 1.5×mmol EDC (Mw=191.71) is added and activated for 20 minutes. After 20 minutes this activated 60PGAPEG1035 solution is added to 10×mmol of Nalpha,Nalpha-biscarboxymethyl-lysine (Mw=262Da) into 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The 60PGAPEG1035DANTA product is filter-sterilized (using 0.2 um polysulfone filter, Nalgene, Rochester, N.Y.) and lyophilized. A 10 mg/ml solution is made of the resulting 60PGAPEG1035DANTA and the hydrodynamic diameter is determined by size exclusion chromatography using a TosohG4000WXL column (0.79×30 cm) and phosphate buffered saline as elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with diameter of approximately 20-40 nm.

### Example 30

Synthesis of 40DXPEG1035DANTA: This composition has dextran or polyglucose (alpha1-6 with alpha1-4 branch; Sigma Chem. Co. St Luis Mo. Cat#31389, Mw=40 kDa) as the polymeric backbone with 35% of the hydroxyl groups derivatized with 10 kDa MPEGAM (Mw=10 kDa MPEGAM has amino group at the terminal; Sunbio, Orinda, Calif.; Cat#P1AM-10) and the remaining hydroxyl groups are occupied with nitrilotriacetic acid. To do this the dextran hydroxyl groups are converted to carboxyl groups. To add carboxyl groups, the hydroxyl groups in dextran can be converted to carboxy groups according to the protocol by Tijsen et al. (Carbohydrate Polymers 2001, vol 45; p 219-226). Briefly, 10 gram of dextran is suspended in 100 ml of 90% propanol/water and add 4 grams NaOH pellet and stirred overnight at 40° C. The next day 10 grams of sodium-monochloro-acetate (Sigma Chem Co. St Luis, Mo. Cat#291773) is added as outlined in Tijsen et al. (Carbohydrate Polymers 2001, vol 45; p 219-226). At the end of the reaction the carboxymethelated dextran is washed with 15 changes of water in a 3 kDa-MWCO ultrafiltration cartridge (UFP-3-E-5A). The resulting product (DX) is filter-sterilized using a 0.2 um polysulfone filter (Nalgene, Rochester, N.Y.) and lyophilized to obtain carboxymethylated dextran. Before synthesis of 40DXPEG1035DANTA, the starting carboxyl group (Xmmol) of 1.0 g DX is measured according to the protocol by Kobayahi and Chiba (Analytical biochemistry 1994, vol 219, p 189-194). To synthesize 40DXPEG1035DANTA, 1.0 g of DX (with Xmmol carboxyl groups) is dissolved in 50 ml of 20 mM MES (2-(N-morpholino)ethanesulfonic acid, Pierce, Rockford, Ill.) buffer pH 4.7 to obtain a DX solution. 0.35×mmol of MPEGAM (Mw=10 kDa MPEG with amino group at the terminal; Sunbio, Orinda, Calif.; Cat#P1AM-10) is dissolved in 20 ml of 80% ethanol and added to the DX solution to make a DXPEG solution. To the DXPEG solution, Xmmol of NHS (Mw=115.14) and Xmmol EDC (Mw=191.71) are added to the solution while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If pH is below 7.1 the pH is adjusted with 10N NaOH one drop at a time to reach 7.1. The reaction to is allowed to proceed for 2 hours and the remaining amino group of the MPEGAM is measured by TNBS and is expected to be none indicating that all 0.35×mmol amino groups is used up and conjugated to the PGA. If there are remaining amino groups in MPEGAM, the pH is adjusted to 5 with 6N HCl and Xmmol EDC (Mw=191.71) is added, and after 20 minutes adjusted back to a pH of 7.1 and allowed to react overnight to form 40DXPEG1035. An aliquot of the resulting 40DXPEG1035 solution is used to determine the hydrodynamic diameter by size exclusion chromatography using TosohG4000WXL column (0.79×30 cm) and phosphate buffered saline (PBS; 11.9 mM phosphate, 137 mM NaCl, 2.7 mMKPO4, pH 7.4) containing 15% Acetonitrile as elution solvent at a flow rate of 0.6 ml/min. The retention time is expected to be consistent with diameter of approximately 14-20 nm. The pH is adjusted down to 5 with 6N HCl and 1.5×mmol EDC (Mw=191.71) is added and activated for 20 minutes. After 20 minutes this activated 40DXPEG1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture containing 40DXPEG1035DANTA is concentrated to 100 ml and washed with 15 changes of water in a 100 kDa-MWCO ultrafiltration cartridge (UFP-100-E-5A). The 40DXPEG1035DANTA product is filter-sterilized using a 0.2 um polysulfone filter (Nalgene, Rochester, N.Y.) and lyophilized. 10 mg/ml solution of resulting 40DXPEG1035DANTA is made and used to determine the hydrodynamic diameter by size exclusion chromatography using TosohG4000WXL column (0.79×30 cm) and elution at a flow rate of 0.6 ml/min with phosphate buffered saline (PBS; 11.9 mM phosphate, 137 mM NaCl, 2.7 mMKPO4, pH 7.4) containing 15% Acetonitrile. The retention time is expected to be consistent with diameter of approximately 17-25 nm.

### Example 31

Synthesis of 31: Sysnthsis of 30CHIPEG1035DAEDTA: This composition has chitosan (Sigma Chem. Co. St Luis Mo. Cat#448869, Mw=35 kDa) as the polymeric backbone with 35% of the amino groups occupied with 10 kDa MPEGCM MPEGCM (Mw=10 kDa; MethoxyPEG with carboxyl groups at the terminal, from Lysan bio; Arab, Ala.) and the remaining amino groups occupied with ethyenediaminetetraacetic acid (EDTA). To synthesize 30CHIPEG1035DAEDTA, 1.0 g of chitosan hydrochloride (with Xmmol aminogroup as measured by TNBS) is dissolved in 25 ml of 1M HEPES buffer at pH 7.4 (Pierce, Rockford, Ill.) to obtain a 30CHI solution. In a separate container, 0.35×mmol of MPEGCM (Mw=10 kDa; MethoxyPEG with carboxyl groups at the terminal, from Lysan bio; Arab, Ala.) is dissolved in 60 ml of 80% ethanol with 20 mM MES pH=4.7 (1200 ul of 1M MES added to 60 ml), 0.5×mmol of NHS (Mw=115.14) is added, and once dissolved Xmmol EDC (mw=191.71; 10.43 mmol) is added while stirring and allowing the activation to proceed for 20 minutes. Activated MPEGCM is added directly to the 30CHI solution, allowing for the reaction to occur for 2 hours and amino groups measured by TNBS to ensure if 35% saturation of amino groups else more of the appropriate amount of activated MPEGCM is added. This is the 30CHIPEG1035DA solution. Size exclusion chromatography is performed using TosohG4000WXL column (0.79×30 cm) with phosphate buffered saline (PBS; 11.9 mM phosphate, 137 mM NaCl, 2.7 mMKPO₄, pH 7.4) containing 15% Acetonitrile elution at a flow rate of 0.6 ml/min. The retention time of is expected to be consistent with approximately 14-17 nm molecular diameter. 5×mmol EDTA dianhydride is added (Mw=256; Sigma Chem Co., St Louis, Mo. Cat#332046) followed by 200 uL TEA. The reaction is titrated slowly with 10 N NaOH to pH 7.1 and stirred for 4 hours. Using TNBS reaction, it is confirmed that no amino groups remain, indicative of a complete reaction, and that the 30CHIPEG1035DAEDTA product is made. Size exclusion chromatography is performed using a TosohG4000WXL column (0.79×30 cm) with phosphate buffered saline (PBS; 11.9 mM phosphate, 137 mM NaCl, 2.7 mMKPO₄, pH 7.4) containing 15% Acetonitrile elution at a flow rate of 0.6 ml/min. The retention time is expected to be consistent with 17-21 nm molecular diameter. The resulting 30CHIPEG1035DAEDTA is washed with 15 volumes of water using a 100,000 MWCO ultrafiltration cartridge (UFP-100-E-5A; GE-Amersham) and lyophilized.

### Examples of the Use of Compositions with a Polymeric Backbone without Modifiable Functional Groups

### Example 32

Use of polyglycine, polyalanine, polyvaline, phenylalanine, polyoxyethyleneglycol, polyoxypropyleneglycol, and similar structures (designated in this example as INRT as a group) as a polymeric backbone is possible by the use of non-specific photoreactive heterobifunctional crosslinkers that can introduce carboxyl functional groups throughout the polymers. Examples of such photoreactive heterobifunctional crosslinkers includes NHS-diazirine (Succinimidyl 4,4′-azipentanoate), NHS-LC-diazirine (Succinimidyl 6-(4,4′-azipentanamido)hexanoate), NHS-55-diazirine (Succinimidyl 2-([4,4′-azipentanamido]ethyl)-1,3′-dithioproprionate), Sulfo-NHS-diazirine (Sulfosuccinimidyl 4,4′-azipentanoate), Sulfo-NHS-LC-diazirine (Sulfosuccinimidyl 6-(4,4′-azipentanamido)hexanoate), Sulfo-NHS-SS-diazirine (Sulfosuccinimidyl 2-([4,4′-azipentanamido]ethyl)-1,3′-dithioproprionate), ANB-NOS(N-5-Azido-2-nitrobenzoyloxysuccinimide), NHS-ASA (N-Hydroxysuccinimidyl-4-azidosalicylic acid), SADP (N-Succinimidyl (4-azidophenyl)-1,3′-dithioproprionate), Sulfo-SAND (Sulfosuccinimidyl-2-(m-azido-o-nitrobenzamido)-ethyl-1,3′-proprionate), SANPAH (N-Succinimidyl-6-(4′-azido-2′-nitrophenylamino) hexanoate), Sulfo-HSAB (N-Hydroxysulfosuccinimidyl-4-azidobenzoate), Sulfo-NHS-LC-ASA (Sulfosuccinimidyl[4-azidosalicylamido]-hexanoate), Sulfo-SADP (N-Sulfosuccinimidyl(4-azidophenyl)-1,3′-dithioproprionate), Sulfo-SAED (Sulfosuccinimidyl 2-[7-amino-4-methylcoumarin-3-acetamido]ethyl-1,3′ dithiopropionate), Sulfo-SANPAH (N-Sulfosuccinimidyl-6-(4′-azido-2′-nitrophenylamino)hexanoate), Sulfo-SBED (Sulfo-N-hydroxysuccinimidyl-2-(6-[biotinamido]-2-(p-azido benzamido)-hexanoamido) ethyl-1,3′-dithioproprionate), and Sulfo-SFAD (Sulfosuccinimidyl-(perfluoroazidobenzamido)-ethyl-1,3′-dithioproprionate). These and related reagents are commercially available from Pierce, Rockford, Ill. Photo-reactive reagents are chemically inert reagents that become reactive when exposed to ultraviolet or visible light. The traditional photo-reactive groups in these reagents are aryl azides. When an aryl azide is exposed to UV light, it forms a nitrene group that can initiate addition reactions with double bonds, insertion into C—H and N—H sites, or subsequent ring expansion to react with a nucleophile (e.g., primary amines). The latter reaction path usually dominates when primary amines are present in the sample. Thiol-containing reducing agents (e.g., DTT or 2-mercaptoethanol) must be avoided in the sample solution during all steps before and during photoactivation. These reagents can reduce the azide. The succinimidyl-ester diazirine (SDA) reagents are a class of crosslinkers that combine proven amine-reactive chemistry with an diazirine-based photochemistry for photo-crosslinking to nearly any other functional group. Diazirine-based photocrosslinkers have better photostability than phenyl azide-based photocrosslinkers and are activated with long-wave UV light (330-370 nm). In the synthesis example that will follow, INRT will designate a polymer that has no readily modifiable groups.

### Example 33

Synthesis of 20INRTPEGG1035DANTA: This composition has INRT as the polymeric backbone with 35% of the photo-introduced carboxyl groups linked to 10 kDa MPEGAM (Mw=10 kDa MPEGAM has amino group at the terminal; Sunbio, Orinda, Calif.; Cat#P1AM-10) and the remaining carboxyl groups occupied with nitrilotriacetic acid. To introduce carboxyl groups, 2 g of INRT (20 kDa) is dissolved in 50-100 ml of appropriate solvent, 20-40 mmol of Sulfo-NHS-diazirine is added and the solution exposed to UV light (330-370 nm) for 2-10 minutes. The pH is adjusted to 9 and left at room temperature for 2 hours to cleave the NHS and expose the carboxyl groups for analysis and quality control. The modified product is washed with 15 changes of water in a 3 kDa-MWCO ultrafiltration cartridge (UFP-3-E-5A). The resulting carboxylated product (20INRT) is filter sterilized using a 0.2 um polysulfone filter (Nalgene, Rochester, N.Y.) and lyophilized to obtain 20INRT. Before using 20INRT for synthesis of 20INRTPEGG1035DANTA, the starting carboxyl group is measured (Xmmol) in 1.0 g 20INRT according to the protocol by Kobayahi and Chiba (Analytical biochemistry 1994, vol 219, p 189-194). If there are less than 1 mmol of carboxyl groups in 1 gram of 20INRT, more carboxyl groups are introduced by repeating the above process. To synthesize 20INRTPEGG1035DANTA, 1.0 g of INRT (with Xmmol carboxyl group) is dissolved in 50 ml of 20 mM MES (2-(N-morpholino)ethanesulfonic acid, Pierce, Rockford, Ill.) buffer pH 4.7 to obtain PGA solution. 0.35×mmol of MPEGAM (Mw=10 kDa MPEG with amino group at the terminal; Sunbio, Orinda, Calif.; Cat#P1AM-10) is dissolved in 20 ml of 80% ethanol and added to the PGA solution to make PGAPEG solution. To the PGAPEG solution, Xmmol of NHS (Mw=115.14) and Xmmol EDC (Mw=191.71) solution is added while stirring. The pH is maintained at pH4.7-5.0 with 6N HCl for 20 minutes using HCl. After 20 minutes of activation, the pH is adjusted to 7.1 by adding 10 ml of 1M HEPES, pH 7.4. If the pH is below 7.1 the pH is adjusted with 10N NaOH one drop at a time to reach 7.1. The reaction is allowed to proceed for 2 hours and the remaining amino groups of the MPEGAM are measured by TNBS and is expected to be none indicating that all 0.35×mmol amino group is used up and conjugated to the INRT. If there are remaining amino groups in MPEGAM, the pH is adjusted to 5 with 6N HCl and Xmmol EDC (Mw=191.71) is added, and after 20 minutes adjusted back the pH of 7.1 and allowed to react overnight to form 20INRTPEGG1035. An aliquot of the resulting 20INRTPEGG1035 solution is added the hydrodynamic diameter is determined by size exclusion chromatography using TosohG4000WXL column (0.79×30 cm) and phosphate buffered saline (PBS; 11.9 mM phosphate, 137 mM NaCl, 2.7 mMKPO₄, pH 7.4) containing 15% Acetonitrile as elution solvent at a flow rate of 0.6 ml/min. The retention time is expected to be consistent with diameter of approximately 12-17 nm. The pH is adjusted down to 5 with 6N HCl and 1.5×mmol EDC added (Mw=191.71) and activated for 20 minutes. After 20 minutes this activated 20INRTPEGG 1035 solution is added to 10×mmol of Nalpha,Nalpha-biscarboxymethyl-lysine (Mw=262Da) in to 25 ml of 1M HEPES buffer at pH 7.4 and allowed to react overnight. The reaction mixture is concentrated to 100 ml and washed with 15 changes of water in a 100 kDa-MWCO ultrafiltration cartridge (UFP-100-E-5A). The 20INRTPEGG1035DANTA product is filter-sterilized using a 0.2 um polysulfone filter, Nalgene, Rochester, N.Y.) and lyophilized. A 10 mg/ml solution of resulting 20INRTPEGG1035DANTA is made and the hydrodynamic diameter is determined by size exclusion chromatography using TosohG4000WXL column (0.79×30 cm) and phosphate buffered saline as elution solvent (PBS; 11.9 mM phosphate, 137 mM NaCl, 2.7 mMKPO₄, pH 7.4) containing 15% Acetonitrile at a flow rate of 0.6 ml/min. The retention time is expected to be consistent with diameter of approximately 16-22 nm.

It should be noted that the preceding examples are not to limit the scope of the invention. The use of other backbones without undue experimentation by those skilled in the arts is inherently disclosed in this specification. As in the examples and inherently disclosed in the process shown in the examples, the invention include the use of other backbones such as polyglycerol, natural saccharides, aminated polysaccharides, aminated oligosaccharides, polyamidoamine, polyacrylic acids, polyalcohols, carboxylated polysaccharides, carboxylated oligosaccharides, aminocarboxylated polysaccharides, aminocarboxylated oligosaccharides, carboxymethylated polysaccharides, or carboxymethylated oligosaccharides are meant to be disclosed in this specification. Those backbones mentioned above with repeating carboxyl groups can be activated to react with amine-containing chelating molecules or modified by a small molecule spacer to contain amino groups, and facilitate reaction with carboxyl-containing chelating groups. Those backbones mentioned above with repeating hydroxyl groups can be reacted with chelating molecules or modified to have a small molecule spacer containing a functional group that can facilitate a reaction with a chelating group.

Again, it should be noted that the synthesis examples above that use linear and branched polymeric backbones are not to limit the scope of the invention. It should also be noted that the synthesis examples above that used a bidentate, a tridentate and a tetradentate chelating molecule represented by species IDA, NTA, TACN, and DTPA is not to limit the scope of the invention. Other chelating moieties can be used using the chemistry, known to those skilled in the art. Examples of chelating molecules that can be used without undue experimentation include: 1,4,7,10-tetraazacyclododecane-N,N′,N″,N′″-tetraacetic acid; 1,4,7,10-tetraaza-cyclododecane-N,N′,N″-triacetic acid; 1,4,7-tris(carboxymethyl)-10-(2′-hydroxypropyl)-1,4,7,10-tetraazocyclodecane; 1,4,7-triazacyclonane-N,N′,N″-triacetic acid; 1,4,8,11-tetraazacyclotetra-decane-N,N′,N″,N′″-tetraacetic acid; 1,2-diaminocyclohexane-N,N,N′,N′-tetraacetic acid; bis(aminoethanethiol)carboxylic acid; ethylenediamine-tetraacetic acid (EDTA); ethyleneglycoltetraacetic acid (EGTA); ethylene-bis(oxyethylene-nitrilo)tetraacetic acid; ethylenedicysteine; N-(hydroxyethyl)ethylenediaminetriacetic acid; nitrilodiacetic acid (NDA); triethylenetetraamine-hexaacetic acid (TTHA); bisphosphonate or a polypeptide having the formula: (Aₓ H_{y})ₚ, wherein A is any amino acid residue, H is histidine, x is an integer from 0-6; y is an integer from 1-6; and p is an integer from 2-6.

### Example 34

TNBS Assay for Primary Amino Groups. The assay for primary amino groups, as used in the preceding examples, was adapted from Spadaro et al. (Anal Biochem, vol 96, p 317-321) and modified to fit a 96-well plate. Stock borate buffer (2.5×) containing 0.1M sodium tetraborate, pH 9.2, was prepared by stirring overnight at room temperature followed by filtration through 0.2 um filter (0.2 um polysulfone filter, Nalgene, Rochester, N.Y.). Lysine stock standard (2.34 mg/ml) was prepared and kept frozen until use. Prior to use, the stock was serially diluted with water 100 fold (23.4 ug/ml or 256 uM primary amino groups); 200 fold (128 uM primary amino groups); 400 fold (64 uM primary amino groups); 800 fold (32 uM primary amino groups); 800 fold (32 uM primary amino groups); and 1600 fold (16 uM primary amino groups). These were plated (150 ul/well) in a 96-well plate (Corning transparent flat bottom polystyrene; Fisher) in triplicate including water alone as zero blank. An aliquot of 4 mg/ml substrate (Gly8Fret) before and after modification (Gly8FretS) was diluted 50 fold and plated (150 ul/well) in triplicate into the 96-well plate. TNBS (1M) was diluted 400 fold using 2.5× borate buffer and 100 ul was added to samples in the 96-well plate. After 30 minutes the absorbance at 420 nm was measured using a Chameleon plate reader. The amino groups in the samples were calculated from regression equation of the standard curve (normally close to y=0.005×-0.02; r²=0.999; where y is the absorbance at 420 nm and x is the concentration of primary amino group in uM) ran with the sample.

### Example 35

Testing of the ability of various carriers to bind to an active agent. Incubation mixtures in triplicate were prepared to determine the ability of various carriers to bind peptides and proteins in general. For 2, 10, 20, 50, 100% loading (weight of peptide or active agent ×100/wt of carrier), 250 ul test solutions were prepared in triplicate at the appropriate final buffer concentration (10 mM HEPES, pH 7.3) containing 0.2 mg/ml test peptide or test proteins, and 10, 2, 1, 0.5, and 0.2 mg/ml carrier. Control samples without carriers were also prepared in similar manner. Samples and controls were filtered through a 100 kDa molecular weight cut off centrifugal membrane filter (Ultracel YM-100; Millipore, Bedford, Mass.) by centrifugation at 14,000×g for 1 minute. Free an active agent in the filtrate was analyzed by HPLC. The results of these studies are presented in Table 1.

TABLE 1
Summary of Insulin binding properties of some PGC-MBs (Protected Graft-Metal Bridge Carriers)

| | | | | | % Free |
|---|---|---|---|---|---|
| | | | Size | NH2/mg | Insulin |
| Lot# | Name structures* | Backbone | (nm) | (nmol) | @x % load** |
| 20070927 | 40PLPEG537-IDA-Zn | Polyamino acid | 19 | 0 | |
| 20071101 | 40PLPEG535-DTPA-Zn | Polyamino acid | 19 | 0 | |
| 20071101 | 40PLPEG535-DTPA-IDA-Zn | Polyamino acid | 19 | 0 | |
| 20080124a | 40PLPEG539DA-NTA-Zn | Polyamino acid | 20 | 20 | 2@15% |
| 20080124b | 40PLPEG537DA-NDA-Zn | Polyamino acid | 20 | 10 | 54@15 |
| 20080326 | 20PLPEG570DA-NTA-Zn | Polyamino acid | 17 | 0 | 17@2% |
| 20080411 | 20PLPEG550DA-DTPA-NTA- | Polyamino acid | 15 | 0 | 11@2% |
| 20080416 | 20PLPEG1055DA-NTA-Zn | Polyamino acid | 21 | 0 | 13@2% |
| 20080421a | 20PLPEG1055DA-PEI4-NTA-Zn | Copolymer | 20 | 0 | 6@2% |
| 20080421b | 20PLPEG1055DA-PEI8-NTA | Copolymer | 25 | 0 | 8@2% |
| 20080603c | 20PLPEG550DA-PEI4-NTA-Zn | Copolymer | 21 | 0 | 0@10% |
| 20080604c | 20PLPEG550DA-PEI8-NTA-Zn | Copolymer | 22 | 0 | 0@10% |
| 20080605c | 20PLPEG550DA-PEI12-NTA-Zn | Copolymer | 21 | 0 | 0@10% |
| 20080804b | 18PEI-PEG1030DA-NTA-Zn | Branched polyamine | 19 | 2 | 66@10% |

*The name structures are convention adapted for easy identification of the carrier. In the above table the 40PL and 20PL indicates a backbone of 40 kDa and 20 kDa polylysine, respectively. The following PEG537, and PEG535, indicate 5 kDa MPEGSuccinate attached to 37, and 35% of the total epsilon amino groups of polylysine, respectively. The following PEG537DA, PEG539DA, PEG550DA and PEG570DA, indicate 5 kDa MPEGcarboxyl attached to 37, 39, 50 and 70% of the total epsilon amino groups of polylysine. The PEG1055DA, PEG1040DA, and PEG1030DA indicate 10 kDa MPEGcarboxyl attached to 55, 40, and 30% of the total epsilon amino groups of polylysine. After the PEG portion, the remaining amino groups are further derivatized by chelators such as iminodiacetic acid-Zn (IDA-Zn), diethylenetriaminepentaacetic acid-Zn (DTPA-Zn), nitrilotriacetic acid-Zn (NTA-Zn), nitrilodiacetic acid-Zn (NDA-Zn) via a succinate linker. In some designs, the remaining amino groups after PEG addition were multiplied by attaching 0.4 kDa, 0.8 kDa, or 1.2 kDa polyetheleneimine (PEI4, PEI8, and PEI12 shown in the table) before addition of the chelators as indicated in the table.
**The “x % load” indicates the amount x (weight) of active agent (insulin) expressed as a percent of carrier weight used. The percent free at various level of loading gives a rough approximatelyimation of how well the carrier binds a specific load molecule. This is also considered for quality control purposes. Typically, the lower the free load molecule, the tighter the binding. If the amount of free remains low at higher loading, it usually indicates a high capacity binding. Proper determination of the Kd and capacity is usually performed by binding studies and Scatchard plots, processes well known to those skilled in the art.

### Example 36

Carrier-metal bridge-insulin formulations last 24 hours or longer in vivo

Experiment 1: Ten Sprague Dawley rats were treated once with 150 mg/kg streptozotocin (STZ) to induce diabetes by destruction of Islet-cells. Diabetes was established five days later as evidenced by a blood glucose measurement of over 300 mg/dl in seven rats. For the in vivo efficacy test of different insulin formulation the rats' blood glucose was checked at t=−1 min. At t=0 min an insulin formulation was injected at the dose indicated in Table 2. Blood glucose was checked at t=1, 15, 30, 60 min, and 2, 3, 4, 5, 6, 7, 24 h and for one formulation additionally at 31 h and 48 h. One formulation (2 mg/kg insulin formulated with 40PLPEG539DANTA-Zn (lot#20080124a) at 1.5% loading suppressed the blood glucose in vivo for more than 30 h. A dose of 0.5 mg/kg of the long-acting insulin formulation called insulin-glargine (Lantus®, Sanofi-Aventis, Bridgewater, N.J.) was used for comparison Table 2. Lantus suppressed blood glucose in these rats for 7 h. Additionally, two out of seven rats receiving 0.5 mg/kg Lantus became hypoglycemic during the test with hypoglycemia defined as blood glucose level below 60 mg/dl. None of the 40PLPEG-backbone formulations shown in Table 2 induced hypoglycemia in any of the rats at the doses indicated, except for one single blood glucose reading at 53 mg/dl with one rat using 4 mg/kg insulin formulated with 40PLPEG539DANTA at 15% loading. When insulin formulated in 20PLPEG570DANTA at 2% loading was given at 1 mg/kg, insulin-induced hypoglycemia in five out of seven rats was observed.

Experiment 2: In another set of experiments, 10 Sprague Dawley rats were treated with a single dose of 150 mg/kg STZ to induce diabetes. Five days later diabetes was established in six rats. These rats were injected with different insulin formulations at 1 mg/kg for the data presented in Table 2. Insulin in vivo efficacy lasted longer than 24 h with the 10% loading 20PLPEG1055DA-PEI4-NTA-Zn formulation. Two of the six rats showed only moderate diabetes as indicated by their average random blood glucose of below 300 mg/dl from week four onwards after the STZ injection, while initially having average random blood glucose of about 450 mg/dl. STZ-induced diabetes is most severe in the two weeks after injection and resolves to some extent thereafter. In these two rats all formulations induced hypoglycemia, but not in the other more severely diabetic rats.

TABLE 2
Long acting basal insulin formulation developed using a metal bridge
carrier is better than the commercially available product (Lantus)

| | | | | Time glucose | Time glucose | |
|---|---|---|---|---|---|---|
| | | Insulin | | minimum was | suppression | Hypoglycemia |
| | **% | Dose | Number | reached after | ends after | below |
| Carrier (Lot#) | load | mg/Kg | of Rats | dosing | dosing | 100 mg/dl |
| Lantus (A Commercial | n/a | 0.5 | 7 | 3 hours | 7 hours | Yes |
| Long acting Insulin | | | | | | |
| formulation) | | | | | | |
| 40PLPEG539DANTA | 1.5 | 2 | 4 | 3 hours | 32 hours | No |
| (20080124a) | | | | | | |
| 40PLPEG539DANTA | 15 | 2 | 3 | 3 hours | 7 hours | No |
| (20080124a) | | | | | | |
| 40PLPEG539DANTA | 15 | 4 | 2 | 4 hours | 8 hours | No |
| (20080124a) | | | | | | |
| 40PLPEG539DANTA | 150 | 1 | 4 | 2 hours | 4 hours | No |
| (20080124a) | | | | | | |
| 20PLPEG570DANTA | 2 | 1 | 7 | 1 hour | 8 hours | Yes |
| (20080124a) | | | | | | |
| 20PLPEG1055DAPEI4NTAZn | 2 | 1 | 6 | 2 hours | 18 hours | No |
| (20080421a) | | | | | | |
| 20PLPEG1055DAPEI4NTAZn | 10 | 1 | 6 | 2 hours | Between | No |
| (20080421a) | | | | | 24 & 30 hours | |
| 20PLPEG550DAPEI4NTAZn | 10 | 1 | 6 | 2 hours | Between | No |
| (20080603c) | | | | | 8 &24 hours | |
| 20PLPEG550DAPEI8NTAZn | 10 | 1 | 6 | 2 hours | Between | No |
| (20080604c) | | | | | 8 &24 hours | |
| 20PLPEG550DAPEI12NTAZn | 10 | 1 | 6 | 2 hours | Between | No |
| (20080605c) | | | | | 8 &24 hours | |
| 20PLPEG550DAPEI4NTAZn | 2 | 1 | 6 | 2 hours | Between | No |
| (20080603c) | | | | | 8 &24 hours | |

**The “x % load” indicates the amount x (weight) of active agent (insulin) expressed as percent of carrier weight used. All formulations with PEI maintained glucose average between 100 and 200 mg/dl from the time the glucose minimum was reached after dosing until suppression ended. Those without PEI have a more variable glucose average from the time the glucose minimum was reached after dosing until suppression ended, ranging from 400 mg/dl for those with hypoglycemia such as 20PLPEG570DANTA.

### Example 37

Binding of rhGH to PLPEGNTAZn/Ni is dependent on the presence of chelated metal (see Table 3). This example is presented to show that the chelator attached to the backbone allows for the binding of protein with metal binding domain. In addition metals such as Zinc or Nickel can be used but is not intended to limit the scope of this invention to these metals. In this particular experiment, 500 μug rhGH were mixed with 40 μl radioactively labeled trace amounts of ¹²⁵I-rhGH (concentration −5 mg/ml). A centricon YM100 column was used to remove rhGH aggregates (flow-through collected). Final [rhGH]=3.22 mg/ml. Various amounts of PLPEGNTAZn/Ni were incubated with 20 μg rhGH in a volume of 100 μl. Unbound rhGH was removed on a Centricon YM100 column. The membrane-retained rhGH-PLPEGNTAZn/Ni complex was washed with 100 μl PBS by centrifugation. Radioactivity in the eluate and retentate were determined separately using a gamma counter (Table 3).

TABLE 3
Binding of metal binding protein (rhGH) to the carrier
is dependent on the presence of chelated metal

| | The fraction of rGH | | |
|---|---|---|---|
| Sample, chelate attached to | retained on YM100 | | μg bound minus |
| PLPEGSA and carrier amount | membrane | μg bound | background |
| Membrane control | 0.05 | 1.03 | control |
| PLPEGSANi, 1 mg (lot#20020102) | 0.05 | 1.04 | 0.01 |
| PLPEGSANi, 2 mg (lot#20020102) | 0.06 | 1.29 | 0.25 |
| PLPEGNTAZn, 1 mg (lot#20020105) | 0.11 | 2.26 | 1.22 |
| PLPEGNTAZn, 2 mg (lot#20020105) | 0.25 | 5.05 | 4.02 |
| PLPEGNTANi, 1 mg (lot#20020104) | 0.10 | 2.05 | 1.01 |
| PLPEGNTANi, 2 mg (lot#20020104) | 0.23 | 4.63 | 3.60 |

Non-specific binding to the YM100 membrane surface and binding to succinylated control (PLPEG; lot#20020101) polymers were similar. Ni and Zn complexes of PLPEGNTA showed 12 to 20-fold higher binding (2 mg polymer in the incubation mixture).

### Example 38

Size-exclusion analysis rhGH complexed with PLPEGNTAZn: PLPEGNTAZn (100 μl, 2 mg) was mixed with 100 μg rhGH and analyzed on a size-exclusion HPLC column (SEC-5, Rainin). Fractions were collected and counted using a gamma-counter (FIG. 3). The formation of a complex between the co-polymer and rhGH is evident from a change in the elution pattern (fractions 11-14 contain higher molecular weight complex). The result demonstrates (FIG. 3) that the interaction of chelated metal the metal binding domain of the protein is stable and can survive the gel permeation chromatography involving thousands of re-equilibration (equal to the number of theoretical plates of the column) as the sample passes through the column. Weak interaction can cause the complex to dissociate resulting in unaltered rhGH peak which is not observed in this case.

### Example 39

Construction of a His-tagged Green Fluorescent Protein (GFP) variant. cDNA encoding for humanized GFP isoform was excised from BlueScriptGFP vector using compatible restriction sites. GFP fragment was then subcloned into SalI-KpnI-restricted pHAT10 vector (Clontech) to afford in-frame expression with His-tag (HAT™) from chicken lactate dehydrogenase (KNHLIHRVHKDDHAHAHRK) containing six histidines. Subcloning was performed by ligating the purified GFP fragment with linearized pHAT10 vector using T4 DNA ligase. Ligation reactions were used for E. coli transformation. Several colonies exhibiting bright green fluorescence under the UV light were selected. Bacterial colonies were transferred into LB broth and grown overnight in a volume of 5 ml. This starter culture was then used for infecting 11 of LB medium grown to the density of 0.8 at 600 nm and bacterial culture was centrifuged at 6000 g to isolate bacterial mass. Bacteria were then lysed using B-PER buffer (Pierce) in the presence of 1× protease inhibitors (with no EDTA, Roche Biochemicals). Lysate was cleared by centrifugation at 16000×g (SS-34 Rotor, Sorvall) and the supernatant was combined with washed, pre-equilibrated TALON™ resin (Clontech). The mixture was agitated at 4 C overnight and washed several times with loading buffer (50 mM phosphate, 300 mM NaCl pH 7). Histidine tagged-GFP product was eluted using 100 mM imidazole in 45 mM Na-phosphate, 270 mM NaCl, pH 7). Fluorescent eluate was dialyzed against PBS, pH 7 and analyzed by electrophoresis.

### Example 40

Binding of histidine tagged-GFP to PLPEGNTA and control polymers (see Table 4 below). This example is presented here to demonstrate that a protein can be modified with chelating molecule such as histidine tag to allow it to bind or enhance its binding to the carrier of the present invention. Similar process can be performed with A peptide, a protein, an oligonucleotide, a polynucleotide or a small molecular weight drug. Complex formation between PLPEGNTA copolymer and histidine-tagged GFP was achieved by combining histidine tagged-GFP and Ni²⁺ or Zn²⁺ salts of PLPEGNTA or PLPEGSA (control). After an hour of incubation, the complexes were placed in a YM-50 membrane. Various amounts of PLPEGNTAZn/Ni were incubated with 20 μg histidine tagged-GFP in a volume of 100 μl. Free non-bound histidine tagged-GFP was removed on Centricon YM100. Membrane-retained PLPEGNTAZn/Ni complex was washed three times using 100 μl PBS aliquots by centrifugation. The fluorescence intensities in eluate and retentate were determined using a fluorometer (excitation 475, emission 510 nm). In some experiments, 100% mouse plasma was added to the incubation mixtures and samples were processed as described before.

TABLE 4
Protein can be modified with histidine to bind or to improve
the binding to metal chelate containing carrier.

| Sample | % GFP bound |
|---|---|
| GFP control | 0.002 |
| PLPEGSA Zn control | 0.003 |
| PLPEGNTAZn | 99.68 |
| PLPEGNTANi | 99.52 |

The obtained results indicate the binding of histidine tagged-GFP to metal chelate of PLPEGNTAZn/Ni co-polymer was highly specific (Table 4) and that the association of His Tagged-GFP with similar co-polymer bearing no NTA residues was close to the background.

In the presence of plasma, binding of histidine tagged-GFP was also specific. Binding to NTA-linked co-polymers in the presence of Ni and Zn cations was approximately the same in the presence or in the absence of the plasma. The only detectable non-specific binding levels were detectable in the case of polycationic PLPEG co-polymer (FIG. 4) and this binding was not inhibited by plasma.

### Example 41

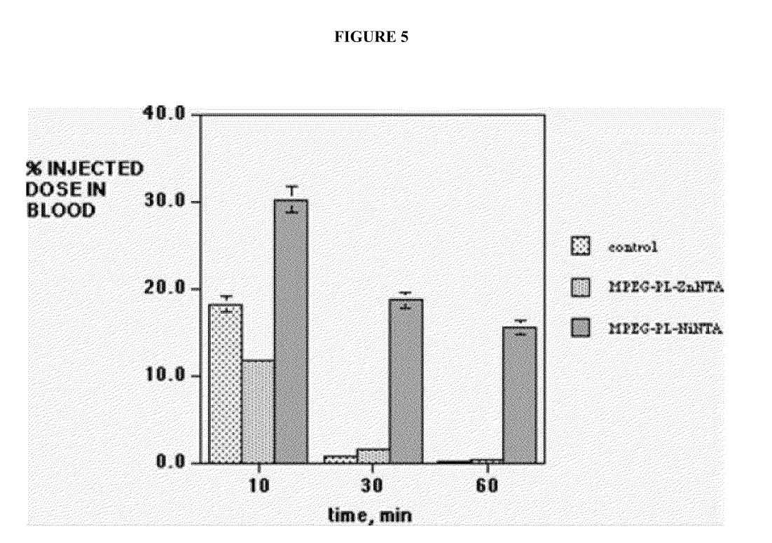

Blood concentration of histidine tagged-GFP and histidine tagged-GFP-PLPEGNTAZn/Ni complexes in vivo after intravenous injection (see FIG. 5). This example is presented to demonstrate that if an active agent is tagged with histidine, the distribution in the blood with time is expected to be similar to this surrogate protein (histidine tagged GFP) and that the presence of the carrier of the present invention can similarly improve the time of residency in the blood. This is also supported by insulin results above. For FIG. 5, pre-formed complexes of histidine tagged-GFP with PLPEGNTANi (lot#20020104) and PLPEGNTAZn (lot#20020105) as well as control histidine tagged-GFP were injected IV in the tail vein of anesthetized balb/c mice (20 μg histidine tagged-GFP mixed with 1 mg of co-polymer or 20 μg histidine tagged-GFP in a total volume of 0.1 ml, 2 per group) and blood samples were drawn through a catheter inserted in a contralateral tail vein. Blood samples (40 μl) were heparinized, centrifuged (3,000 g) and plasma samples were analyzed for histidine tagged-GFP using fluorometry (excitation-475/emission 508 nm). Observed fluorescence intensity values were normalized for injection dose using histidine tagged-GFP standard diluted in mouse plasma. The blood volume was calculated as 7% of animal weight and hematocrit—at 50%.

### Example 42

Other methods for determination of carrier active agent complex formation efficiency. Alternative methods to evaluate the efficiency of binding to the carrier to an active agent includes radioiodination of a peptide, a protein, an oligonucleotide, a polynucleotide or a small molecular weight drug. Radio-iodinated an active agent can be obtained by using sodium [¹²⁵I] iodide in the presence of Iodo-Gen (Pierce) at approximately. 0.01-0.05 mCi/μg peptide followed by purification on C18-reversed phase HPLC column using a gradient of acetonitrile in 0.1% TFA. Due to the possibility of additional histidine radioiodination reactions in the presence and in the absence of trace amounts of Zn to protect the His residue can be performed. The ability of the peptide to form a complex with ZnNTA after the radioiodination can be tested by measuring the retention of radioactivity on Zn-saturated NTA-column. Trace amounts of radioiodinated active agent can be mixed with cold active agent followed by the incubation with Carrier-Zn (PLPEGNTAZn) or to determine complex formation efficiency. Additionally, considering the a peptide, a protein, an oligonucleotide, a polynucleotide or a small molecular weight drug, as purchased, likely has a zinc already present in its active site (AMBI, personal communication), labeled and unlabelled an active agent can also be mixed with carrier without zinc already chelated to the PLPEGNTA. Unbound an active agent can be removed using Microcon YM100-ultrafiltration followed by the separate radioactivity determination in the eluate and the retentate. Alternative method to evaluate the efficiency of binding to the carrier to an active agent includes radioiodination of a peptide, a protein, an oligonucleotide, a polynucleotide or a small molecular weight drug. Radio-iodinated an active agent can be obtained by using sodium [125I] iodide in the presence of Iodo-Gen (Pierce) at approximately. 0.01-0.05 mCi/μg peptide followed by purification on C18-reversed phase HPLC column using a gradient of acetonitrile in 0.1% TFA. Due to the possibility of additional histidine radioiodination reactions in the presence and in the absence of trace amounts of Zn to protect the His residue will be performed. The ability of the peptide to form a complex with ZnNTA after the radioiodination can be tested by measuring the retention of radioactivity on Zn-saturated NTA-column. Trace amounts of radioiodinated an active agent can be mixed with cold an active agent followed by the incubation with Carrier-Zn (PLPEGNTAZn) or to determine complex formation efficiency. Additionally, considering the a peptide, a protein, an oligonucleotide, a polynucleotide or a small molecular weight drug, as purchased, likely has a zinc already present in its active site (AMBI, personal communication), labeled and unlabelled an active agent can also be mixed with carrier without zinc already chelated to the PLPEGNTA. Unbound an active agent can be removed using Microcon YM100-ultrafiltration followed by the separate radioactivity determination in the eluate and the retentate.

### Example 43

Synthesis of CHIPEG1033DANTA: a) chitosan (Sigma cat #44,886-9; 1 g or 5 mmol NH2 by TNBS) will be dissolved in 25 ml of 1 M HEPES. This is the CHI solution. b) In a separate container, 15 g of MPEGCM (Mw=10 kDa; 1.5 mmol; SunBright; ME-100HS; lot#M62503; clear solution) will be dissolved in 60 ml of 80% ethanol with 20 mM MES pH=4.7 (1200 ul of 1M MES added to 60 ml), 250 mg of NHS (mw=115.14; 2.17 mmol) will be added, once dissolved 1.0 g EDC (mw=191.71; 5.2 mmol) will added while stirring. Activation will be allowed to proceed for 20 minutes and the activated MPEGCM will added directly to CHI solution in step a. The pH will be adjusted to pH 7.1 slowly with 10N NaOH one drop at a time, and will be allowed to react for 2 hours. Amino group will be analyzed and expected to be between 1.2 to 2 mmol or 24% to 40% MPEG saturation. This will be the CHIPEG solution. c) Size Exclusion chromatography will be performed using TosohG4000WXL column (0.79×30 cm) will be eluted with phosphate buffered saline (PBS; 11.9 mM phosphate, 137 mM NaCl, 2.7 mMKPO₄, pH 7.4) containing 15% Acetonitrile at a flow rate of 0.6 ml/min to determine retention time and size which is expected to be between 18 nm to 30 nm molecular diameter. d) Succinic Anhydride (2 g; 20 mmol) will be added followed by 200 uL TEA. The reaction will be slowly titrated with 10 N NaOH to pH 7.1 and stirred for 4 hours. The amino groups will be measured and expected to be 0 umol. Size Exclusion chromatography will be performed using TosohG4000WXL column (0.79×30 cm) and will be eluted with phosphate buffered saline (PBS; 11.9 mM phosphate, 137 mM NaCl, 2.7 mMKPO₄, pH 7.4) containing 15% Acetonitrile at a flow rate of 0.6 ml/min to determine retention time and size which is expected to be between 19 nm to 30 nm molecular diameter. The reaction mixture will be washed with 15 volumes of water using a 100,000 MWCO ultrafiltration cartridge (UFP-100-E-5A; GE-Amersham) and will be lyophilized (expected to be about 10 g or more). e) 2 gram of NTA-amine (Nalpha,Nalpha, -Bis(carboxymethyl)-L-Lysine; Mw=262.26+50% impurity, up to 2 mol water and 10% inorganic) or ˜4 mmol will be dissolved in 10 ml of 1M HEPES. Twenty ml of 0.5M ZnCl was added to the NTA-amine and adjusted to pH7.1 with 10N NaOH. The solution will be centrifuged and supernatant will be collected and total amino group will be determined by TNBS (expected to be between 4.0-5.0 mmol). f) CHIPEG1033DASA (3.0 g or about 0.50 mmol carboxyl) will be dissolved in 15 ml of 20 mM MES, 115 mg NHS (Mw=115.09; 1 mmol) will be added, followed by 500 mg EDC (mw=191.71; 2.6 mmol). The pH will go up slowly but will be maintained to 4.7 by HCl. After 20 minutes, CHIPEG1033DASA solution will be added to NTA-amine supernatant and pH will be adjusted to 7.1 using 10N NaOH. After 2 hours, total amino group will be measured by TNBS (expected to be 3.5 mmol or 0.5 mmol NTA-amine incorporation to 3.0 g carrier. g) To remove Zinc, 2 g of NTA (Nitrilotriacetic acid; Mw=191.14) or 10 mmol will be added to solution in f and will be adjusted to pH 7.0 with 10N NaOH, followed by 10 ml of Imidazole (5M) and pH will go up to 8. h) The CHIPEG1033DA-NTA will be washed with 10 volumes of water using a 100,000 MWCO ultrafiltration cartridge (UFP-100-E-5A; GE-Amersham). Sample CHIPEG1033DA-NTA will be filter-sterilized (0.2 um polysulfone filter; Nalgene, Rochester, N.Y.) and lyophilized (expected to be about 2.5 g). Ten mg/ml of CHIPEG1033DA-NTA will be analyzed by Size Exclusion chromatography using TosohG4000WXL column (0.79×30 cm) and will be eluted with phosphate buffered saline (PBS; 11.9 mM phosphate, 137 mM NaCl, 2.7 mMKPO₄, pH 7.4) containing 15% Acetonitrile at a flow rate of 0.6 ml/min. The expected molecular diameter is between 19-30 nm. TNBS analysis of CHIPEG1033DA-NTA is expected to give 0+/−5 uM NH₂ or 0 nmol/mg.

### Example 44

Synthesis of 15PAPEG1033DANTA: a) Polyallylamine hydrochloride (0.5 g or 5 mmol NH2 by TNBS; Sigma cat #283215; Mw=15,000 Daltons) will be dissolved in 25 ml of 1 M HEPES. This is the 15PA solution. b) In a separate container, 15 g of MPEGCM (Mw=10 kDa; 1.5 mmol; SunBright; ME-100HS; lot#M62503; clear solution) will be dissolved in 60 ml of 80% ethanol with 20 mM MES pH=4.7 (1200 ul of 1M MES added to 60 ml), 250 mg of NHS (mw=115.14; 2.17 mmol) will be added, once dissolved 1.0 g EDC (mw=191.71; 5.2 mmol) will be added while stirring. Activation will be allowed to proceed for 20 minutes and the activated MPEGCM will be added directly to 15PA solution in step a. The pH will be adjusted to pH 7.1 slowly with 10N NaOH one drop at a time, and will be allowed to react for 2 hours. Amino group will be analyzed and expected to be between 1.2 to 2 mmol or 24% to 40% MPEG saturation. This will be the 15PAPEG solution. c) Size Exclusion chromatography will be performed using TosohG4000WXL column (0.79×30 cm) will be eluted with phosphate buffered saline (PBS; 11.9 mM phosphate, 137 mM NaCl, 2.7 mMKPO₄, pH 7.4) containing 15% Acetonitrile at a flow rate of 0.6 ml/min to determine retention time and size which is expected to be between 15 nm to 25 nm molecular diameter. d) Succinic Anhydride (2 g; 20 mmol) will be added followed by 200 uL TEA. The reaction will be slowly titrated with 10 N NaOH to pH 7.1 and stirred for 4 hours. The amino groups will be measured and expected to be 0 umol. Size Exclusion chromatography will be performed using TosohG4000WXL column (0.79×30 cm) and will be eluted with phosphate buffered saline (PBS; 11.9 mM phosphate, 137 mM NaCl, 2.7 mMKPO₄, pH 7.4) containing 15% Acetonitrile at a flow rate of 0.6 ml/min to determine retention time and size which is expected to be between 19 nm to 30 nm molecular diameter. The reaction mixture will be washed with 15 volumes of water using a 100,000 MWCO ultrafiltration cartridge (UFP-100-E-5A; GE-Amersham) and will be lyophilized (expected to be about 10 g or more). e) 2 gram of NTA-amine (Nalpha,Nalpha, -Bis(carboxymethyl)-L-Lysine; Mw=262.26+50% impurity, up to 2 mol water and 10% inorganic) or ˜4 mmol will be dissolved in 10 ml of 1M HEPES. Twenty ml of 0.5M ZnCl was added to the NTA-amine and adjusted to pH7.1 with 10N NaOH. The solution will be centrifuged and supernatant will be collected and total amino group will be determined by TNBS (expected to be between 4.0-5.0 mmol). f) 15PAPEG1033DASA (3.0 g or about 0.50 mmol carboxyl) will be dissolved in 15 ml of 20 mM MES, 115 mg NHS (Mw=115.09; 1 mmol) will be added, followed by 500 mg EDC (mw=191.71; 2.6 mmol). The pH will go up slowly but will be maintained to 4.7 by HCl. After 20 minutes, 15PAPEG1033DASA solution will be added to NTA-amine supernatant and pH will be adjusted to 7.1 using 10N NaOH. After 2 hours, total amino group will be measured by TNBS (expected to be 3.5 mmol or 0.5 mmol NTA-amine incorporation to 3.0 g carrier. g) To remove Zinc, 2 g of NTA (Nitrilotriacetic acid; Mw=191.14) or 10 mmol will be added to solution in f and will be adjusted to pH 7.0 with 10N NaOH, followed by 10 ml of Imidazole (5M) and pH will go up to 8. h) The 15PAPEG1033DA-NTA will be washed with 10 volumes of water using a 100,000 MWCO ultrafiltration cartridge (UFP-100-E-5A; GE-Amersham). Sample 15PAPEG1033DA-NTA will be filter-sterilized (0.2 um polysulfone filter; Nalgene, Rochester, N.Y.) and lyophilized (expected to be about 2.5 g). Ten mg/ml of 15PAPEG1033DA-NTA will be analyzed by Size Exclusion chromatography using TosohG4000WXL column (0.79×30 cm) and will be eluted with phosphate buffered saline (PBS; 11.9 mM phosphate, 137 mM NaCl, 2.7 mMKPO₄, pH 7.4) containing 15% Acetonitrile at a flow rate of 0.6 ml/min. The expected molecular diameter is between 15-25 nm. TNBS analysis of 15PAPEG1033DA-NTA is expected to give 0+/−5 uM NH2 or 0 nmol/mg.

While preferred embodiments of the present invention have been shown and described herein, it will be obvious to those skilled in the art that such embodiments are provided by way of example only. Numerous variations, changes, and substitutions will now occur to those skilled in the art without departing from the invention. It is expected to be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a polymeric backbone comprising monomeric units;
a chelating group covalently bonded to a monomeric unit in the polymeric backbone;
a protective chain covalently bonded to a monomeric unit in the polymeric backbone;
a transition metal ion chelated to the chelating group; and
an active agent with a metal binding domain coordinately bonded to the transition metal ion.

2. The composition of claim 1 wherein the polymeric backbone comprises a polysaccharide.

3. The composition of claim 1 wherein the polymeric backbone comprises a polyamino acid.

4. The composition of claim 1 wherein the protective chain is different from the polymeric backbone.

5. The composition of claim 1, wherein the protective side chain comprises poly(ethyleneglycol).

6. The composition of claim 1, wherein the protective side chain comprises alkoxy poly(ethyleneglycol).

7. The composition of claim 1, wherein the protective side chain comprises methoxy poly(ethyleneglycol).

8. The composition of claim 1 and wherein the protective side chain has a molecular weight of between 1,000 to 20,000 Daltons.

9. The composition of claim 1, wherein the polymeric backbone comprises a linear polymer.

10. The composition of claim 1, wherein the polymeric backbone comprises a branched polymer.

11. The composition of claim 1, wherein the polymeric backbone comprises a co-polymer made up of at least two polymers.

12. The composition of claim 1, wherein the polymeric backbone comprises a polymer selected from the group consisting of polylysine, polyornithine, polyarginine, polyaspartic acid, polyglutamic acid, polyserine, polythreonine, polycysteine, polyglycerol, polyethyleneimine, polyallylamine, chitosan, hyluronan, natural saccharide, aminated polysaccharide, aminated oligosaccharide, polyamidoamine, polyacrylic acid, polyalcohol, carboxylated polysaccharide, carboxylated oligosaccharide, aminocarboxylated polysaccharide, aminocarboxylated oligosaccharide, carboxymethylated polysaccharide, and carboxymethylated oligosaccharide.

13. The composition of claim 1, wherein the polymeric backbone has repeating nitrogen groups.

14. The composition of claim 13, wherein the polymeric backbone comprises a polyallyamine.

15. The composition of claim 13, wherein the polymeric backbone comprises a polyethyleneimine.

16. The composition of claim 13, wherein the polymeric backbone comprises a chitosan.

17. The composition of claim 13, wherein the polymeric backbone comprises a polylysine.

18. The composition of claim 13, wherein the polymeric backbone is co-polymer made up of at least two polymers selected from a group consisting of polylysine, polyornithine, polyarginine, polyethyleneimines, polyallylamine, chitosan, aminated polysaccharides, aminated oligosaccharides, polyamidoamine.

19. The composition of claim 13, wherein the polymeric backbone is co-polymer comprising of polylysine and polyethyleneimine.

20. The composition of claim 1, wherein the chelating group or the metal binding domain is selected from one or more of the following:
1,4,7,10-tetraazacyclododecane-N,N′,N″,N′″-tetraacetic acid;
1,4,7,10-tetraaza-cyclododecane-N,N′,N″-triacetic acid;
1,4,7-tris(carboxymethyl)-10-(2′-hydroxypropyl)-1,4,7,10-tetraazocyclodecane;
1,4,7-triazacyclonane-N,N′,N″-triacetic acid;
1,4,8,11-tetraazacyclotetra-decane-N,N′,N″,N′″-tetraacetic acid;
1,2-diaminocyclohexane-N,N,N′,N′-tetraacetic acid;
bis(aminoethanethiol)carboxylic acid;
diethylenetriamine-pentaacetic acid (DTPA);
ethylenediamine-tetraacetic acid (EDTA);
ethyleneglycoltetraacetic acid (EGTA);
ethylene-bis(oxyethylene-nitrilo)tetraacetic acid;
ethylenedicysteine;
Imidodiacetic acid (IDA);
N-(hydroxyethyl)ethylenediaminetriacetic acid;
nitrilotriacetic acid (NTA);
nitrilodiacetic acid (NDA);
bisphosphonate;
triethylenetetraamine-hexaacetic acid (TTHA);
Trimethyl-1,4,7-triazacyclononane (TACN);
2,3-Dimercaptopropanol (BAL);
Meso-2,3-Dimercaptosuccinic acid (DMSA);
Monoisoamyl meso-2,3-dimercaptosuccinic acid (Mi-ADMS);
Sodium 2,3-dimercapto-1-propanesulfonate (DMPS);
Cyclohexanediaminetetraacetic acid (CDTA);
D-Penicillamine (DPA);
N-acetylcysteine (NAC);
2-Mercaptopropionyl glycine (Tiopronin);
Sodium 4,5-dihydroxybenzene-1,3-disulfonate (Tiron);
Desferrioxamine (deferoxamine, DFO);
1,2-Dimethyl-3-hydroxypyridin-4-one (deferiprone, L1); or
Triethylene tetramine (Trientine, Trien).

21. The composition of claim 1, wherein the chelating group or the metal binding domain comprises a polypeptide having the formula: (AₓH_{y})ₚ, wherein A is any amino acid residue, H is histidine, x is an integer from 0-6; y is an integer from 1-6; and p is an integer from 2-6.

22. The composition of claim 7, wherein the chelating group or metal binding domain is IDA.

23. The composition of claim 7, wherein the chelating group or metal binding domain is NDA.

24. The composition of claim 7, wherein the chelating group or metal binding domain is bisphosphonate.

25. The composition of claim 7, wherein the chelating group is NTA.

26. The composition of claim 7, wherein the chelating group is TACN.

27. The composition of claim 7, wherein the chelating group is combined DTPA and NTA.

28. The composition of claim 7, wherein the polymeric backbone has repeating carbonyl groups

29. The composition of claim 28, wherein polymeric backbone comprises a polysaccharide

30. The composition of claim 28, wherein polymeric backbone comprises a polyacrylic acid.

31. The composition of claim 28, wherein polymeric backbone is polyaspartate.

32. The composition of claim 28, wherein polymeric backbone is glutamate.

33. The composition of claim 1, wherein the polymeric backbone is co-polymer made up of at least two polymers selected from a group consisting of from polyaspartic acid, polyglutamic acid, hyluronan, polyacrylic acids, carboxylated polysaccharides, carboxylated oligosaccharides, aminocarboxylated polysaccharides, aminocarboxylated oligosaccharides, carboxymethylated polysaccharides, or carboxymethylated oligosaccharides.

34. The composition of claim 1, wherein the transition metal ion is one or more of the following: Zn²⁺, Ni²⁺, Co²⁺, Fe²⁺, Mn²⁺, or Cu²⁺.

35. The composition of claim 1, wherein the transition metal ion is Zn²⁺, Cu²⁺ or Ni²⁺.

36. The composition of claim 1, wherein the metal ion is Zn²⁺.

37. The composition of claim 1, wherein the active agent comprises any one active agent from the group consisting of: a peptide, a protein, an oligonucleotide, a polynucleotide, a peptidomimetic, a deoxyribonucleic acid, a ribonucleic acid, a nucleic acid derivative, an oligosaccharide, a polysaccharide, a proteoglycan, or a small organic molecule.

38. The composition of claim 1, wherein the active agent comprises any one active agent from the group consisting of: factor VII, factor VIII, insulin, growth factors, hormones, nerve growth factors, brain derived neurotrophic factor, enzymes, endostatin, angiostatin, trombospondin, urokinase, and interferon.

39. The composition of claim 37, wherein the active agent further comprises a covalently bonded chelating group.

40. The composition of claim 7, wherein the active agent is insulin.

41. A pharmaceutical composition of comprising any one of the compositions in claims 1, 7, and 37.

42. A method of prolonging the blood circulation time of an active agent comprising a metal binding domain wherein the method comprises administering the composition of claim 1 to a subject in need thereof.

43. A method of treating a subject diagnosed with diabetes and in need of treatment thereof, the method comprising administering the pharmaceutical composition of claim 39 to the subject.
